(19) United States
(12) Patent Application Publication
(10) Pub. No.: US 2005/0245588 A1
(43) Pub. Date: Nov. 3, 2005

(54) Octahydro-2-h-naphtho[1,2-f]indole-4-carboxamide derivatives as selective glucocorticoid receptor modulators

(76) Inventors: Amjad Ali, Piscataway, NJ (US); Susan D. Aster, Teaneck, NJ (US); James M. Balkovec, Martinsville, NJ (US); Donald W. Graham, Mountainside, NJ (US); Julianne A. Hunt, Scotch Plains, NJ (US); Florida Kallashi, Yonkers, NY (US); Peter J. Sinclair, Scotch Plains, NJ (US); James R. Tata, Westfield, NJ (US); Gayle E. Taylor, Jersey City, NJ (US); Joung L. Goulet, Westfield, NJ (US)

Correspondence Address: MERCK AND CO., INC, P O BOX 2000, RAHWAY, NJ, 07065-0907, US

(21) Appl. No.: 10/527,615

(22) PCT Filed: Sep. 17, 2003
(86) PCT No.: PCT/US03/29494

§ 371 Date: Mar. 11, 2005

Related U.S. Application Data
(60) Provisional application No. 60/412,231, filed on Sep. 20, 2002.
(60) Provisional application No. 60/476,079, filed on Jun. 5, 2003.

(51) Int. Cl. C07D 231/54; A61K 31/4162

(52) U.S. Cl. 514/406; 548/358.5

(57) ABSTRACT

The present invention encompasses compounds of Formula I: or pharmaceutically acceptable salts or hydrates thereof, which are useful as selective glucocorticoid receptor ligands for treating a variety of autoimmune and inflammatory diseases or conditions. Pharmaceutical compositions and methods of use are also included.

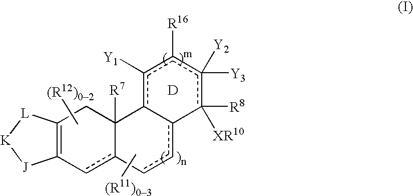

### BACKGROUND OF THE INVENTION

Intracellular receptors (IR's) are a class of structurally related proteins involved in the regulation of gene expression. The steroid hormone receptors are a subset of this superfamily whose natural ligands are typically comprised of endogenous steroids such as estradiol, progesterone, and cortisol. Man-made ligands to these receptors play an important role in human health and, of these receptors, the glucocorticoid receptor has an essential role in regulating human physiology and immune response. Steroids that interact with the glucocorticoid receptor have been shown to be potent antfinflammatory agents. The present invention is directed to a novel class of compounds that are selective glucocorticoid receptor modulators that have potent ani-inflammatory and immunosupresive activity and possess advantages over steroidal glucocorticoid ligands with respect to side effects, efficacy, toxicity and/or metabolism.

### SUMMARY OF THE INVENTION

The present invention encompasses compounds of Formula I: 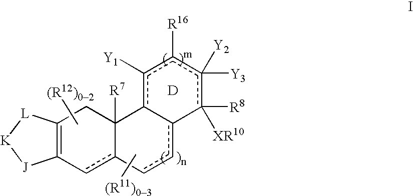
or pharmaceutically acceptable salts or hydrates thereof, which are useful as selective glucocorticoid receptor ligands for treating a variety of autoimmune and inflammatory diseases or conditions. Pharamaceutical compositions and methods of use are also included.

### DETAILED DESCRIPTION OF THE INVENTION

The present invention encompasses compounds of Formula I 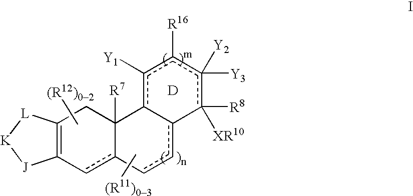
or a pharmaceutically acceptable salt or hydrate thereof, wherein:

n and m are each independently 0, 1 or 2;

J is selected from NR¹ or C(R¹)(R²);

K is selected from NR³ or C(R³)(R⁴);

L is selected from NR⁵ or C(R⁵)(R⁶);

X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);

k is0, 1 or 2;

R¹ and R¹⁰ are each independently selected from the group consisting of:

(1) C₁₋₆alkyl,

(2) C₂₋₆alkenyl,

(3) C₂₋₆akynyl,

(4) C₃₋₆cycloalkyl,

(5) C₁₋₆alkoxy,

(6) C₁₋₆alkyl-S(O)ₖ—, wherein k is 0, 1 or 2,

(7) aryl,

(8) aryl C₁₋₆alkyl,

(9) HET,

(10) —C₁₋₆alkyl-HET,

(11) aryloxy,

(12) aroyloxy,

(13) aryl C₂₋₆alkenyl,

(14) aryl C₂₋₆alkynyl,

(15) hydrogen,

(16) hydroxyl and

(17) cyano,

wherein items (1) to (6) above and the alkyl portions of items (8) and (10) above and the alkenyl portion of item (13) above and the alkynyl portion of item (14) above are optionally substituted from one up to the maximum number of substitutable positions with a substituent independently selected from the group consisting of: halo, oxo, OR¹³, N(R¹⁴)₂, C₃₋₆cycloalkyl and C₁₋₆alkyl-S(O)ₖ—, wherein k is 0, 1 or 2, and

wherein items (7), (9), (11) and (12) above and aryl portion of items (8), (13) and (14) above and the HET portion of item (10) above are optionally substituted from one up to the maximum number of substitutable positions with a substituent independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy,
(o) CN and
(p) C₃₋₆cycloalkyl,
   wherein items (d) to (g) and (p) above and the alkyl portions of item (k) above are optionally substituted from one up to the maximum number of substitutable positions with a substituent independently selected from the group consisting of: halo, OR¹³ and N(R¹⁴)₂, and
   wherein items (h), (i), (j), (l) and (m) above and the aryl portions of items (k) and (n) above are optionally substituted from one up to the maximum number of substitutable positions with a substituent independently selected from the group consisting of: halo, OR¹³ and C₁₋₄alkyl,

R², R³, R⁴, R⁵ and R⁶ are each independently selected from the group consisting of:

(1) hydrogen,

(2) halo,

(3) C₁₋₆alkyl,

(4) C₂₋₆alkenyl,

(5) C₂₋₆akynyl,

(6) C₃₋₆cycloalkyl,

(7) C₁₋₆alkoxy,

(8) C₁₋₆alkyl-S(O)ₖ—, wherein k is 0, 1 or 2,

(9) aryl,

(10) aryl C₁₋₆alkyl,

(11) HET and

(12) —C₁₋₆alkyl-HET,

wherein items (3) to (8) above and the alkyl portions of items (10) and (12) above are optionally substituted from one up to the maximum number of substitutable positions with a substituent independently selected from the group consisting of: halo, OR¹³, N(R¹⁴)₂ and C₁₋₆alkyl-S(O)ₖ—, wherein k is 0, 1 or 2; and

wherein items (9) and (11) and the aryl portion of items (10) and the HET portion of item (12) are optionally substituted from one up to the maximum number of substituable positions with a substituent independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl and
(g) C₁₋₆alkyl-S(O)ₖ—, wherein k is 0, 1 or 2,
   wherein items (d) to (g) above are optionally substituted with from one up to the maximum number of substitutable positions with a substituent independently selected from the group consisting of: halo, OR¹³ and N(R¹⁴)₂,
   or R¹ and R³ or R³ and R⁵ may be joined together to form a double bond;

R⁷ is selected from the group consisting of:

(1) hydrogen,

(2) OR¹³,

(3) C₁₋₄alkyl,

(4) aryl and

(5) aryl C₁₋₄alkyl,

wherein item (3) above and the alkyl portion of item (5) above are optionally substituted with from one up to the maximum number of substitutable positions with a substituent independently selected from the group consisting of: halo, OR¹³ and N(R¹⁴)₂, and

wherein item (4) above and the aryl portion of item (5) above are optionally substituted with from one up to the maximum number of substitutable positions with a substituent independently selected from the group consisting of:

(a) halo,
(b) OR¹³,
(c) N(R¹⁴)₂,
(d) C₁₋₆alkyl,
(e) C₂₋₆alkenyl and
(f) C₂₋₆akynyl,
   wherein items (d) to (f) above are optionally substituted with from one up to the maximum number of substitutable positions with a substituent independently selected from the group consisting of: halo, OR¹³ and N(R¹⁴)₂;
   each Y₁, Y₂ and Y₃ are independently selected from the group consisting of:

(1) hydrogen,

(2) —O—R⁹,

(3) —S(O)ₖ—R⁹, wherein k is 0, 1 or 2,

(4) —C—W—R9, wherein W is O or S(O)k,

(5) —N(R¹⁵)₂,

(6) —S(O)ₖ—N(R¹⁵)₂,

(7) —N(R¹⁵)—S(O)k—N(R¹⁵)₂,

(8) NO₂,

(9) —C(O)—R¹⁵,

(10) —C(O)O—R¹⁵,

(11) —CN,

(12) halo,

(13) —O—S(O)ₖ—R¹⁵ and

(14) C₁₋₄alkyl, optionally substituted with with from 1 to 6 halo groups,

with the proviso that when Y₂ is hydrogen, Y₃ is —C(O)—R¹⁵, R¹⁵ is C₁₋₆alkyl and X is —C(O) then R¹⁰ is not C₁₋₆alkyl, and

with the proviso that when Y₂ is —C(O)—R¹⁵, Y₃ is hydrogen, R¹⁵ is C₁₋₆alkyl and X is —C(O) then R¹⁰ is not C₁₋₆alkyl, and

with the proviso that when Y₂ and Y₃ are both hydrogen, X is a bond and R¹⁰ is HET, then said HET is defined as a 5-membered aromatic or non-aromatic monocyclic ring containing 1-3 heteroatoms selected from O, S and N,

R⁸ is selected from the group consisting of: hydrogen, C₁₋₆alkyl, C₁₋₆alkoxy, —C₁₋₆alkyl-C(O)OH and —C₁₋₆alkyl-C(O)O—C₁₋₆alkyl, wherein the C₁₋₆alkyl portion is optionally mono, di or tri substituted with halo; or where R⁸ and —XR¹⁰ together with the carbon atom to which they are attached form the spiro group: 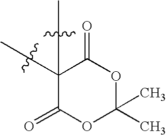
R⁹ is selected from the group consisting of: hydrogen, C₁₋₁₂alkyl and aryl, wherein C₁₋₁₂alkyl and aryl are optionally substituted from one up to the maximum number of substituents with halo;
each R¹¹, R¹² and R¹⁶ is independently selected from the group consisting of:

(1) hydrogen,

(2) halo,

(3) C₁₋₆alkyl,

(4) C₂₋₆alkenyl,

(5) C₁₋₆alkoxy and

(6) hydroxy,

wherein items (3) to (5) above are optionally substituted from one up to the maximum number of substitutable positions with a substituent independently selected from the group consisting of: halo, OR¹², N(R¹³)₂ and C₁₋₆alkyl-S(O)ₖ—, wherein k is 0, 1 or 2,

or R¹⁶ may additionally be hydrogen;

each R¹³ and R¹⁴ is independently selected from the group consisting of hydrogen and C₁₋₄alkyl, optionally substituted from one up to the maximum number of substitutable positions with halo; and

each R¹⁵ is independently selected from the group consisting of: hydrogen, C₁₋₆alkyl, aryl and C₁₋₁₂alkoxycarbonyl, wherein said C₁₋₆alkyl and C₁₋₁₂alkoxycarbonyl are optionally substituted from one up to the maximum number of substituable positions with halo and said aryl is optionally substituted from one up to the maximum number of substituable positions with halo and C₁₋₄alkyl, optionally substituted with 1-3 halo groups.

In an alternative embodiment XR¹⁰ may be CN.

The optional double bond shown in ring A of the compound of Formula I is depicted as a dotted line and means that the double bond may or may not be present. This is illustrated below for a sub-set of compounds within Formula I:

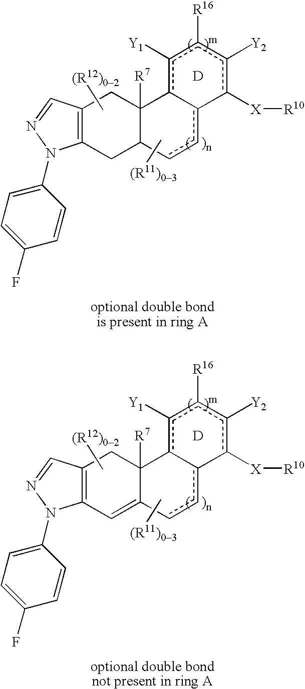

The substituent R¹² in Formula I may or may not be present. When present, one or two R¹² groups may occupy the positions illustrated below: 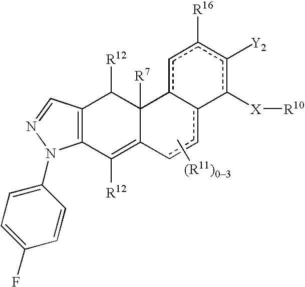
Two R¹² groups may also reside on the same carbon atom.

The substituent R¹¹ in Formula I may or may not be present. When present, one, two or three R¹¹ groups may occupy the positions illustrated below: 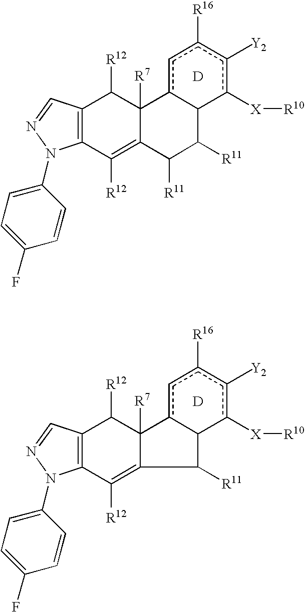
Two R¹¹ groups may also reside on the same carbon atom.

The optional double bonds show in ring D of the compound of
Formula I may occupy the positions illustrated below:

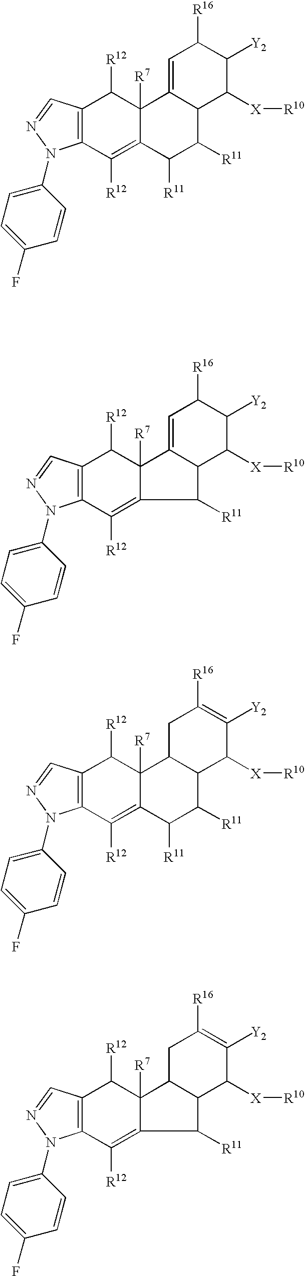

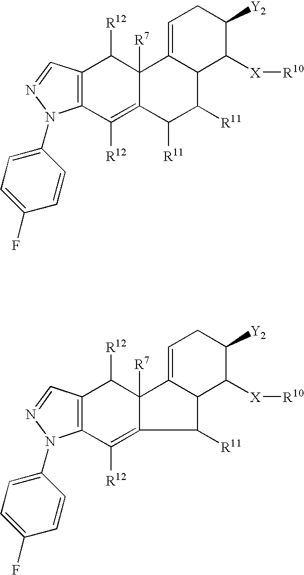

An embodiment of the invention encompasses a genus of compounds of Formula I wherein:

J is NR¹;

K is NR³;

L is C(R⁵)(R⁶); and

R³ and R⁵ are joined together to form a double bond.

An embodiment of the invention encompasses a genus of compounds of Formula I, herein identified as compounds of Formula Ia:

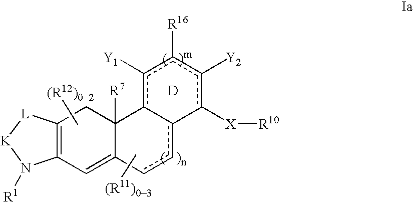

Within the genus of compounds of Formula Ia is the sub-genus of compounds wherein:

R¹ is phenyl or pyridyl said phenyl or pyridyl or optionally mono or di-substituted with a substituent independently selected from the group consisting of:

(a) halo,
(b) OCH₃,
(d) CH₃,
(e) CN.

Within this sub-genus of compounds is the class of compounds wherein R¹ is phenyl, optionally mono or di-substituted with halo.

Within the genus of compounds of Formula Ia are the compounds wherein Y₁ is hydrogen.

Also within the genus of compounds of Formula Ia are the compounds wherein R¹⁶ is hydrogen.

Also within the genus of compounds of Formula Ia are the compounds wherein R⁷ is methyl.

Also within the genus of compounds of Formula Ia are the compounds wherein Y₁ is hydrogen and R¹² is hydrogen.

An embodiment of the invention encompasses compounds of Formula I and Formula Ia wherein:

X is a bond, —C(O), —N(R¹⁴)—, —N(R¹⁴)—C(O)—, —C(O)—N(R¹⁴)—, —N(R¹⁴)—C(O)—NH—; and

R¹⁴ is hydrogen or methyl.

An embodiment of the invention encompasses compounds of Formula I and Formula Ia wherein:

X is a bond, —C(O), —N(R¹⁴)—, —N(R¹⁴)—C(O)—, —C(O)—N(R¹⁴)—, —N(R¹⁴)—C(O)—NH—;

Y₁ is hydrogen;

R¹ is phenyl, optionally mono or di-substituted with halo;

R⁷ is methyl.

R¹¹ is hydrogen;

R¹² is hydrogen;

R¹⁴ is hydrogen or methyl;

R¹⁶ is hydrogen; and

R¹⁰ are each independently selected from the group consisting of:

(1) C₁₋₄alkyl,

(2) C₂₋₄alkenyl,

(3) C₂₋₄akynyl,

(4) C₃₋₆cycloalkyl,

(5) C₁₋₄alkoxy,

(6) aryl,

(7) aryl C₁₋₄alkyl,

(8) HET,

(9) —C₁₋₄alkyl-HET,

(10) aryloxy,

(11) aroyloxy,

(12) aryl C₂₋₄alkenyl,

(13) aryl C₂₋₆alkynyl,

wherein items (1) to (5) above and the alkyl portions of items (7) and (9) above and the alkenyl portion of item (12) above and the alkynyl portion of item (13) above are optionally substituted with from one to three substituents independently selected from the group consisting of: halo, OR¹³, N(R¹⁴)₂, C₃₋₆cycloalkyl and C₁₋₆alkyl-S(O)ₖ—, wherein k is 0, 1 or 2, and

wherein items (6), (8), (10) and (11) above and aryl portion of items (7), (12) and (13) above and the HET portion of item (9) above are optionally substituted with from one to three substituents independently selected from the group consisting of:

(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
   wherein items (d) to (f) above and the alkyl portions of item (i) above are optionally substituted from with one to three substituents independently selected from the group consisting of: halo, OR¹³ and N(R¹⁴)₂, and
   wherein items (g), (h), (j) and (k) above and the aryl portions of items (i) and (l) above are optionally substituted with from one to three substituents independently selected from the group consisting of: halo, OR¹³ and C₁₋₄alkyl,

Another embodiment of the invention encompasses a genus of compounds of Formula I, herein identified as compounds of Formula Ib: 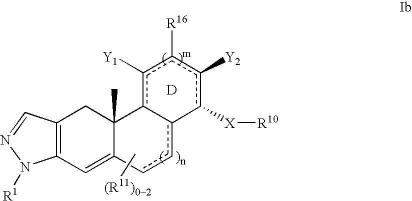
wherein:
m is 0 or 1,
n is 0 or 1,
R¹ is phenyl, optionally mono or di-substituted with halo;
R¹⁰ are each independently selected from the group consisting of:

(1) C₁₋₆alkyl,

(2) C₂₋₆alkenyl,

(3) C₂₋₆akynyl,

(4) C₃₋₆cycloalkyl,

(5) C₁₋₆alkoxy,

(6) C₁₋₆alkyl-S(O)ₖ—, wherein k is 0, 1 or 2,

(7) aryl,

(8) aryl C₁₋₆alkyl,

(9) HET,

(10) —C₁₋₆alkyl-HET,

(11) aryloxy,

(12) aroyloxy,

(13) aryl C₂₋₆alkenyl,

(14) aryl C₂₋₆alkynyl,

(15) hydrogen, and

(16) hydroxy

wherein items (1) to (6) above and the alkyl portions of items (8) and (10) above and the alkenyl portion of item (13) above and the alkynyl portion of item (14) above are optionally substituted with from one to three substituents independently selected from the group consisting of: halo, OR¹³, N(R¹⁴)₂, C₃₋₆cycloalkyl and C₁₋₆alkyl-S(O)ₖ—, wherein k is 0, 1 or 2, and

wherein items (7), (9), (11) and (12) above and aryl portion of items (8), (13) and (14) above and the HET portion of item (10) above are optionally substituted with from one to three substituents independently selected from the group consisting of:

(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy and
(o) CN,
   wherein items (d) to (g) above and the alkyl portions of item (k) above are optionally substituted from one to three substituents independently selected from the group consisting of: halo, OR¹³ and N(R¹⁴)₂, and
   wherein items (h), (i), (j), (l) and (m) above and the aryl portions of items (k) and (n) above are optionally substituted with from one to three substituents independently selected from the group consisting of: halo, OR¹³ and C₁₋₄alkyl,
   each R¹³ and R¹⁴ is independently selected from the group consisting of hydrogen and C₁₋₄alkyl, optionally substituted from one to three halo groups;
   R¹⁶ and each R¹¹ are independently selected from the group consisting of:

(1) hydrogen,

(2) halo,

(3) methyl,

(4) methoxy, and

(5) hydroxy;

Y₁ and Y₂ are each selcected from the group consisting of:

(1) hydrogen,

(2) hydroxy,

(3) halo,

(4) methyl,

(5) —NO₂,

(6) —CN,

(7) mono, di or tri halo substituted methyl,

X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);

Within Formula Ib, there is the sub-genus of compounds wherein Y₁, R¹¹ and R¹⁶ are each hydrogen.

Within Formula Ib, there is a sub-genus of compounds herein identified as Formula Ic: 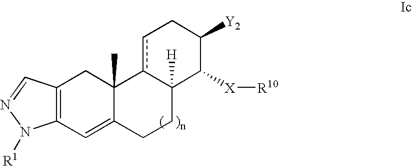
wherein
n is 0 or 1,
R¹ is phenyl, optionally mono or di-substituted with halo;
R¹⁰ is selected from the group consisting of:

(1) C₁₋₆alkyl,

(2) C₂₋₆alkenyl,

(3) C₂₋₆akynyl,

(4) C₃₋₆cycloalkyl,

(5) C₁₋₆alkoxy,

(6) C₁₋₆alkyl-S(O)ₖ—, wherein k is 0, 1 or 2,

(7) aryl,

(8) aryl C₁₋₆alkyl,

(9) HET,

(10) —C₁₋₆alkyl-HET,

(11) aryloxy,

(12) aroyloxy,

(13) aryl C₂₋₆alkenyl,

(14) aryl C₂₋₆alkyl,

(15) hydrogen, and

(16) hydroxy

wherein items (1) to (6) above and the alkyl portions of items (8) and (10) above and the alkenyl portion of item (13) above and the alkynyl portion of item (14) above are optionally substituted with from one to three substituents independently selected the group consisting of: halo, OR¹³, N(R¹⁴)₂, C₃₋₆cycloalkyl and C₁₋₆alkyl-S(O)ₖ—, wherein k is 0, 1 or 2, and

wherein items (7), (9), (11) and (12) above and aryl portion of items (8), (13) and (14) above and the HET portion of item (10) above are substituted with from one to three substituents independently selected from the group consisting of:

(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy and
(o) CN,
   wherein items (d) to (g) above and the alkyl portions of item (k) above are optionally substituted with from one to three substituents independently selected from the group consisting of: halo, OR¹³ and N(R¹⁴)₂, and
   wherein items (h), (i), (j), (l) and (m) above and the aryl portions of items (k) and (n) above are optionally substituted with from one to three substituents independently selected from the group consisting of: halo, OR¹³ and C₁₋₄alkyl,
   each R¹³ and R¹⁴ is independently selected from the group consisting of hydrogen and C₁₋₄alkyl, optionally substituted with from one up to three halo groups;
   R¹⁶ and each R¹¹ are independently selected from the group consisting of:

(1) hydrogen,

(2) halo,

(3) methyl,

(4) methoxy, and

(5) hydroxy;

Y₁ and Y₂ are each selcected from the group consisting of:

(1) hydrogen,

(2) hydroxy,

(3) halo,

(4) methyl,

(5) —NO₂,

(6) —CN,

(6) mono, di or tri halo substituted methyl,

X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);

Within this sub-genus of Formula Ic there is a class of compounds wherein:

X is a bond, —C(O), —N(R¹⁴)—, —N(R¹⁴)—C(O)—, —C(O)—N(R¹⁴)—, —N(R¹⁴)—C(O)—NH—;

R¹³ and R¹⁴ are each independently selected from hydrogen or methyl; and

R¹⁰ are each independently selected from the group consisting of:

(1) C₁₋₄alkyl,

(2) C₂₋₄alkenyl,

(3) C₂₋₄akynyl,

(4) C₃₋₆cycloalkyl,

(5) C₁₋₄alkoxy,

(6) aryl,

(7) aryl C₁₋₄alkyl,

(8) HET,

(9) —C₁₋₄alkyl-HET,

(10) aryloxy,

(11) aroyloxy,

(12) aryl C₂₋₄alkenyl,

(13) aryl C₂₋₆alkynyl,

wherein items (1) to (5) above and the alkyl portions of items (7) and (9) above and the alkenyl portion of item (12) above and the alkynyl portion of item (13) above are optionally substituted with from one to three substituents independently selected from the group consisting of: halo, OR^{13,} N(R¹⁴)₂, C₃₋₆cycloalkyl and C₁₋₆alkyl-S(O)ₖ—, wherein k is 0, 1 or 2, and

wherein items (6), (8), (10) and (11) above and aryl portion of items (7), (12) and (13) above and the HET portion of item (9) above are optionally substituted from one up to the maximum number of substitutable positions with a substituent independently selected from the group consisting of:
(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
   wherein items (d) to (f) above and the alkyl portions of item (i) above are optionally substituted from one up to the maximum number of substitutable positions with a substituent independently selected from the group consisting of: halo, OR¹³ and N(R¹⁴)₂, and
   wherein items (g), (h), (j) and (k) above and the aryl portions of items (i) and (l) above are optionally substituted with from one to three substituents independently selected independently selected from the group consisting of: halo, OR¹³ and C₁₋₄alkyl.

Within this class of compounds of Formula Ic is a sub-class of compounds wherein:

X is a bond, —C(O), —N(R¹⁴)—, —N(R¹⁴)—C(O)—, —C(O)—N(R¹⁴)—, —N(R¹⁴)—C(O)—NH—;

R¹³ and R¹⁴ are each independently from hydrogen or methyl; and

R¹⁰ are each independently selected from the group consisting of:

(1) C₃₋₆cycloalkyl,

(2) aryl,

(3) aryl C₁₋₄alkyl,

(4) HET,

(5) —C₁₋₄alkyl-HET,

(6) aryl C₂₋₄alkenyl,

wherein item (1) above and the alkyl portions of items (3) and (5) above and the alkenyl portion of item (8) above are optionally substituted with from one to three substituents independently selected from the group consisting of: halo, OR¹³, N(R¹⁴)₂, and

wherein the aryl portion of items (2), (3), (6) and the HET portion of item (4) and (5) above are optionally substituted with from one to three substituents independently selected from the group consisting of:

(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
   wherein items (d) to (f) above and the alkyl portions of item (i) above are optionally substituted with from one to three substituents independently selected from the group consisting of: halo, OR¹³ and N(R¹⁴)₂, and
   wherein items (g), (h), (j) and (k) above and the aryl portions of items (i) and (l) above are optionally substituted with from one to three substituents independently selected from the group consisting of: halo, OR¹³ and C₁₋₄alkyl.

Within this subclass of compounds of Formula Ic are the compounds wherein:

R¹⁰ are each independently selected from the group consisting of:

(1) C₃₋₆cycloalkyl,

(2) aryl,

(3) aryl C₁₋₄alkyl,

(4) HET,

(5) —C₁₋₄alkyl-HET,

(6) aryl C₂₋₄alkenyl,

wherein item (1) above and the alkyl portions of items (3) and (5) above and the alkenyl portion of item (8) above are optionally substituted with from to three substituents independently selected from the group consisting of: halo, OR¹³, N(R¹⁴)₂, and

wherein the HET portion of item (4) and (5) are optionally substituted with from one to three substituents selected from the group consisting of C₁₋₄alkyl and aryl, and

wherein the aryl portion of items (2), (3), (6) above are optionally substituted with from one to three substituents independently selected from the group consisting of:

(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
   wherein items (d) to (f) above and the alkyl portions of item (i) above are optionally substituted from one up to the maximum number of substitutable positions with a substituent independently selected from the group consisting of: halo, OR¹³ and N(R¹⁴)₂, and
   wherein items (g), (h), (j) and (k) above and the aryl portions of items (i) and (l) above are optionally substituted from one up to the maximum number of substitutable positions with a substituent independently selected from the group consisting of: halo, OR¹³ and C₁₋₄alkyl.

Another embodiment of the invention encompasses a compound of Formula Ia wherein Y₂ is CF₃. Within this embodiment R10 is selected from the group consisting of:

(1) phenyl,

(2) benzyl, and

(3) HET, wherein HET is a 5-membered aromatic or non-aromatic monocyclic ring containing 1-3 heteroatoms selected from O, S and N,

wherein groups (1) to (3) above are optionally substituted with 1 to 3 substituents independently selected from the group consisting of:

(a) halo,
(b) C₁₋₄alkyl, optionally substituted with hydroxy or 1 to 3 halo groups,
(c) C₁₋₄alkoxy, optionally substituted with 1 to 3 halo groups,
(d) NH₂,
(e) hydroxy, and
(e) phenyl or benzyl.

Another embodiment of the invention encompasses a compound of Formula Ia wherein Y₂ is hydrogen, X is a bond and R¹⁰ is HET, wherein HET is a 5-membered aromatic or non-aromatic monocyclic ring containing 1-3 heteroatoms selected from O, S and N. Within this embodiment HET is selected from oxazolyl and imidazolyl.

Another embodiment of the invention encompasses a compound of Formula Id 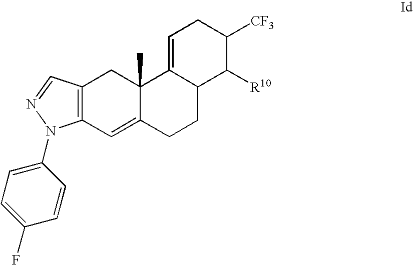
or a pharmaceutically acceptable salt thereof, wherein
R¹⁰ is a 5-membered aromatic or non-aromatic mono-cyclic ring containing 1-3 heteroatoms selected from O, S, and N, and
R¹⁰ is mono-substituted with phenyl, wherein phenyl is optionally substituted with 1-3 substituents independently selected from halo, C₁₋₄alkyl and C₁₋₄alkoxy. Within this embodiment R¹⁰ is oxazolyl, oxadiazolyl or thiazolyl. Also within this embodiment R¹⁰ is oxazolyl.

Another embodiment of the invention encompasses a pharmaceutical composition comprising a compound of Formula I in combination with a pharmaceutically acceptable carrier.

Another embodiment of the invention encompasses a method for treating a glucocorticoid receptor mediated disease or condition in a mammalian patient in need of such treatment comprising administering the patient a compoud of Formula I in an amount that is effective for treating the glucocorticoid receptor mediated disease or condition.

Within this embodiment is encompassed the above method wherein the glucocorticoid receptor mediated disease or condition is selected from the group consisting of: tissue rejection, leukemias, lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity, metabolic syndrome, inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, inflammatory scalp alopecia, panniculitis, psoriasis, discoid lupus erythematosus, inflamed cysts, atopic dermatitis, pyoderma gangrenosum, pemphigus vulgaris, buflous pernphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma, Human Immunodeficiency Virus (HIV), cell apoptosis, cancer, Kaposi's sarcoma, retinitis pigmentosa, cognitive performance, memory and learning enhancement, depression, addiction, mood disorders, chronic fatigue syndrome, schizophrenia, sleep disorders, and anxiety.

Another embodiment of the invention encompasses a method of selectively modulating the activation, repression, agonism and antagonism effects of the glucocorticoid receptor in a mammal comprising administering to the mammal a compound of Formula I in an amount that is effective to modulate the glucocorticoid receptor.

Exemplifying the invention are the compounds of the Examples disclosed hereunder.

The invention is described using the following definitions unless otherwise indicated.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl, cyclopropyl, cyclobutyl, cyclopentyl and cyclohexyl.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkylthio” means alkylthio groups having the indicated number of carbon atoms of a straight, branched or cyclic configuration. C₁₋₆alkylthio, for example, includes methylthio, propylthio, isopropylthio, and the like.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes , propenyl, 1-methylethenyl, butenyl and the like.

The term “cycloalkyl” means mono-, bi- or tri-cyclic structures, optionally combined with linear or branched structures, the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, and the like.

The term “aryl” is defined as a mono- or bi-cyclic aromatic ring system and includes, for example, phenyl, naphthyl, and the like.

The term “aralkyl” means an alkyl group as defined above of 1 to 6 carbon atoms with an aryl group as defined above substituted for one of the alkyl hydrogen atoms, for example, benzyl and the like.

The term “aryloxy” means an aryl group as defined above attached to a molecule by an oxygen atom (aryl-O) and includes, for example, phenoxy, naphthoxy and the like.

The term “aralkoxy” means an aralkyl group as defined above attached to a molecule by an oxygen atom (aralkyl-O) and includes, for example, benzyloxy, and the like.

The term “arylthio” is defined as an aryl group as defined above attached to a molecule by an sulfur atom (aryl-S) and includes, for example, thiophenyoxy, thionaphthoxy and the like.

The term “aroyl” means an aryl group as defined above attached to a molecule by an carbonyl group (aryl-C(O)—) and includes, for example, benzoyl, naphthoyl and the like.

The term “aroyloxy” means an aroyl group as defined above attached to a molecule by an oxygen atom (aroyl-O) and includes, for example, benzoyloxy or benzoxy, naphthoyloxy and the like.

The term “HET” is defined as a 5- to 10-membered aromatic, partially aromatic or non-aromatic mono- or bicyclic ring, containing 1-4 heteroatoms selected from O, S and N, and optionally substituted with 1-2 oxo groups. Preferably, “HET” is a 5- or 6-membered aromatic or non-aromatic monocyclic ring containing 1-3 heteroatoms selected from O, S and N, for example, pyridine, pyrimidine, pyridazine, furan, thiophene, thiazole, oxazole, isooxazole and the like, or HET is a 9- or 10-membered aromatic or partially aromatic bicyclic ring containing 1-3 heteroatoms selected from O, S, and N, for example, benzofuran, benzothiophene, indole, pyranopyrrole, benzopyran, quionoline, benzocyclohexyl, naphtyridine and the like. “HET” also includes the following: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

For all of the above definitions, each reference to a group is independent of all other references to the same group when referred to in the Specification. For example, if both R¹ and R² are HET, the definitions of HET are independent of each other and R¹ and R² may be different HET groups, for example furan and thiophene.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician; The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

The following abbreviations have the indicated meanings:

AIBN=2.2′-azobisisobutyronitrile

B.P.=benzoyl peroxide

Bn=benzyl

CCl₄=carbon tetrachloride

D=—O(CH₂)₃O—

DAST=diethylamine sulfur trifluoride

DCC=dicyclohexyl carbodiimide

DCI=1-(3-dimethylaminopropyl)-3-ethyl carbodiimide

DEAD=diethyl azodicarboxylate

DIBAL=diisobutyl aluminum hydride

DME=ethylene glycol dimethylether

DMAP=4-(dimethylamino)pyridine

DMF=N,N-dimethylformamide

DMSO=dimethyl sulfoxide

Et₃N=triethylamine

LDA=lithium diisopropylamide

m-CPBA=metachloroperbenzoic acid

NBS=N-bromosuccinimide

NSAID=non-steroidal anti-inflammatory drug

PCC=pyridinium chlorochromate

PDC=pyridinium dichromate

Ph=phenyl

1,2-Ph=1,2-benzenediyl

Pyr=pyridinediyl

Qn=7-chloroquinolin-2-yl

R^{S}=—CH₂SCH₂CH₂Ph

r.t.=room temperature

rac.=racemic

THF=tetrahydrofuran

THP=tetrahydropyran-2-yl

Alkyl Group Abbreviations

Me=methyl

Et=ethyl

n-Pr=normal propyl

i-Pr=isopropyl

n-Bu=normal butyl

i-Bu=isobutyl

s-Bu=secondary butyl

t-Bu=tertiary butyl

c-Pr=cyclopropyl

c-Bu=cyclobutyl

c-Pen=cyclopentyl

c-Hex=cyclohexyl

Some of the compounds described herein contain one or more asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumarid, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that in the discussion of methods of treatment which follows, references to the compounds of Formula I are meant to also include the pharmaceutically acceptable salts.

The magnitude of prophylactic or therapeutic dose of a compound of Formula I will, of course, vary with the nature and the severity of the condition to be treated and with the particular compound of Formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for oral administration to humans may contain from about 0.5 mg to about 5 g of active agent compounded with an appropriate and convenient amount of carrier material which may vary from about 5 to about 95 percent of the total composition. Dosage unit forms will generally contain from about 1 mg to about 2 g of an active ingredient, typically 25 mg, 50 mg, 100 mg, 200 mg, 300 mg, 400 mg, 500 mg, 600 mg, 800 mg, or 1000 mg.

For the treatment of glucocorticoid receptor mediated diseases the compound of Formula I may be administered orally, topically, parenterally, by inhalation spray or rectally in dosage unit formulations containing conventional non-toxic pharmaceutically acceptable carriers, adjuvants and vehicles. The term parenteral as used herein includes subcutaneous, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, solutions, aqueous or oily suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing a compound of Formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

The ability of the compounds of Formula I to selectively modulate glucocorticoid receptors makes them useful for treating, preventing or reversing the progression of a variety of inflammatory and autoimmune diseases and conditions. Thus, the compounds of the present invention are useful to treat, prevent or ameliorate the following diseases or conditions: inflammation, tissue rejection, auto-immunity, various malianancies, such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity and metabolic syndrome.

The compounds of the present invention are also useful for treating, preventing or reversing the progression of disease states involving systemic inflammation such as inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, and cirrhosis.

The compounds of the present invention are useful for treating, preventing or reversing the progression of a variety of topical diseases such as inflammatory scalp alopecia, panniculitis, psoriasis, discoid lupus erythematosus, inflamed cysts, atopic dermatitis, pyoderma gangrenosum, pemphigus vulgaris, buflous pernphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma.

The compounds of the present invention are also useful in treating, preventing or reversing the progression of disease states associated with Human Immunodeficiency Virus (HIV), cell apoptosis, and cancer including, but not limited to, Kaposi's sarcoma, immune system activation and modulation, desensitization of inflammatory responses, IIL-I expression, natural killer cell development, lymphocytic leukemia, and treatment of retinitis pigmentosa. Cogitive and behavioral processes are also susceptible to glucocorticoid therapy where antagonists would potentially be useful in the treatment of processes such as cognitive performance, memory and learning enhancement, depression, addiction, mood disorders, chronic fatigue syndrome, schizophrenia, stroke, sleep disorders, and anxiety.

The invention also encompasses a method for treating a glucocorticoid receptor mediated disease comprising concomitantly administering to a patient in need of such treatment a compound of Formula I and one or additional more agents. For treating or preventing asthma or chronic obstructive pulmonary disease, the compounds of Formula I may be combined with one or more agents selected from the group consisting of: [symbol] -agonists (e.g., salmeterol), theophylline, anticholinergics (e.g., atropine and ipratropium bromide), cromolyn, nedocromil and leukotriene modifiers (e.g., montelukast). For treating or preventing inflammation, the compounds of Formula I may be combined with one or the following: a salicylate, including acetylsalicylic acid, a non-steroidal antiinflammatory drug, including indomethacin, sulindac, mefenamic, meclofenamic, tolfenamic, tolmetin, ketorolac, dicofenac, ibuprofen, naproxen, fenoprofen, ketoprofen, flurbiprofin and oxaprozin, a TNF inhibitor, including etanercept and infliximab, an IL-1 receptor antagonist, a cytotoxic or immunosuppressive drug, including methotrexate, leflunomide, azathioprine and cyclosporine, a gold compound, hydroxychloroquine or sulfasalazine, penicillamine, darbufelone, and a ρ38 kinase inhibitor. The compound of Formula I may also be used in combination with bisphonates such as alendronate to treat a glucocorticoid mediated disease and simultaneously inhibit osteoclast-mediated bone resorption.

### METHODS OF SYNTHESIS

Generally, compounds of the present invention may be synthesized by following the following synthetic scheme:

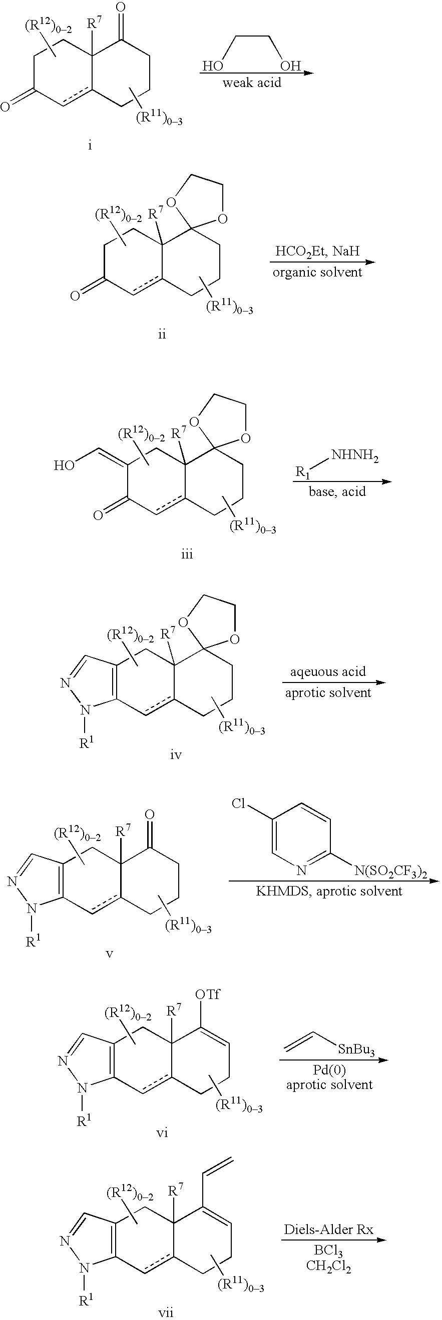

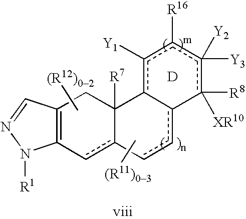

Acid, such as p-toluenesulfonic acid, is added to a solution of the Wieland-Miescher ketone i in ethylene glycol to give ketal ii. Ethyl formate and sodium hydride are added to ketal ii in an organic solvent such as anhydrous benzene to afford hydroxyketone iii. The hydroxyketone iii is dissolved in an appropriate acid such as glacial acetic acid and the appropriate hydrazine such as p-fluorophenylhyradzine hydrocloride and appropriate base such as sodium acetate is added to give pyrazole ketal iv. The pyrazole ketal iv is dissolved in an aprotic solvent such as THF and an aqeuous acid such as aqeuous 6N HCl is added to yield the ketone v.

2-[N,N-Bis(trifluomethylsulphonyl)-amino]-5-chloropyridine is added to Potassium bis(trimethylsilyl amide) in an aprotic solvent such as THF to give the triflate vi. Diene vii is obtained by reaction of vi with vinyl tributyl tin and a palladium (0) source such as tetrakis(triphenylphosphine) palladium (0) in an aprotic solvent such as THF. Diels-Alder reaction in the presence of a Lewis acid in a non-polar solvent such as methylene chloride at low temperature yields the final product viii.

Methods for making compounds of Formula I outside the scope of formula viii are easily discernible by those having ordinary skill in the art in view of the above method and the examples set for the below. See, for example, Org. React. 4, 1, 1948; Org. React. 4, 60, 1958; Chem. Rev. 61, 537, 1961; Diels-Alder Reaction, Elsevier, London, 1965;1-4 Cycloadditions Reactions—The Diels-Alder Reaction in Heterocyclic Syntheses, Academic Press, New York, 1967; Angew Chem. Int. Ed. Engl. 19, 779, 1980; J. Am. Chem. Soc. 95, 4094, 1973.

The invention will now be illustrated by the following non-limiting examples in which, unless stated otherwise:

(i) all operations were carried out at room or ambient temperature, that is, at a temperature in the range 18-25° C.,

(ii) evaporation of solvent was carried out using a rotary evaporator under reduced pressure (600-4000 pascals: 4.5-30 mm. Hg) with a bath temperature of up to 60° C.,

(iii) the course of reactions was followed by thin layer chromatography (TLC) and reaction times are given for illustration only;

(iv) melting points are uncorrected and ‘d’ indicates decomposition; the melting points given are those obtained for the materials prepared as described; polymorphism may result in isolation of materials with different melting points in some preparations;

(v) the structure and purity of all final products were assured by at least one of the following techniques: TLC, mass spectrometry, nuclear magnetic resonance (NMR) spectrometry or microanalytical data;

(vi) yields are given for illustration only;

(vii) when given, NMR data is in the form of delta (δ) values for major diagnostic protons, given in parts per million (ppm) relative to tetramethylsilane (TMS) as internal standard, determined at 500 MHz or 600 MHz using the indicated solvent; conventional abbreviations used for signal shape are: s. singlet; d. doublet; t. triplet; m. multiplet; br. broad; etc.: in addition “Ar” signifies an aromatic signal;

(viii) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq (equivalent(s)).

### EXAMPLE 1

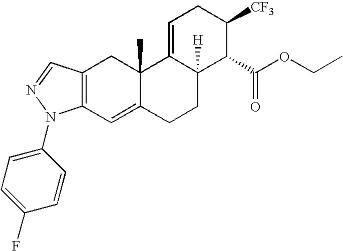

### Ethyl (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylate

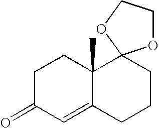

### Step A: (8aR)-8a-hydroxy-3,4,8,8a-tetrahydronaphthalene-1,6(2H,7H)-dione-1-ethylene ketal

(S) Wieland-Miescher ketone (5 g, 28.05 mmol) was dissolved in ethylene glycol (140 mL) and stirred at room temperature. Next, 4 Å molecular sieves (˜5 g) followed by p-toluenesulfonic acid (5.34 g, 28.05 mmol) were added. After stirring at room temperature for 23 minutes, the reaction was poured slowly into a 2:1 mixture of ice water/sat. NaHCO₃ (150 mL). The reaction was extracted with EtOAc (4×100 mL) and the combined organic layers were washed with brine (100 mL), dried over MgSO₄, filtered and concentrated in vacuo. The residue was purified by flash chromatography (0 to 40% EtOAc/hexanes) on silica gel to afford 5.77 g (93%) of (8aR)-8a-hydroxy-3,4,8,8a-tetrahydronaphthalene-1,6(2H,7H)-dione-1-ethylene ketal as a white solid. LCMS=223; (M+1)⁺. ¹H NMR (CDCl₃, 500 MHz): δ 5.83 (br d, J=1.8 Hz, 1H), 4.43-3.94 (m, 4H), 2.49-2.40 (m, 3H), 2.39-2.27 (m, 2H), 1.95-1.88 (m, 1H), 1.84-1.78 (m, 1H), 1.76-1.64 (m, 3H), 1.37 (s, 3H).

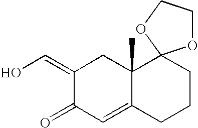

### Step B: (7E,8aS)-7-(hydroxymethylene)-8a-methyl-3,4,8,8a-tetrahydronaphthalene-1,6(2H,7H)-dione-1-ethylene ketal

(8aR)-8a-hydroxy-3,4,8,8a-tetrahydronaphthalene-1,6(2H,7H)-dione-1-ethylene ketal (4.8 g, 21.62 mmol) was dissolved in 200 mL benzene and ˜50 mL benzene were distilled off atmospherically. The solution was cooled to −40° C. and ethyl formate (7.36 mL, 86.48 mmol) was added, followed by sodium hydride (60% suspension in mineral oil; 3.46 g, 86.48 mmol). Next, 450 mL MeOH was added (bubbling evident) and the reaction was allowed to warm to room temperature. After stirring for 3 hours, the reaction was cooled to 0° C. and 50 mL H₂O was added slowly. The biphasic system was shaken and the organic layer was washed with H₂O (3×50 mL). The combined aqueous layers were washed with diethyl ether (100 mL) and then acidified to pH 5.5-6 with sat. KH₂PO₄. The aqueous layer was extracted with EtOAc (5×200 mL). The combined extracts were dried over Na₂SO₄ and concentrated in vacuo to afford 5.04 g (93%) of (7E,8aS)-7-(hydroxymethylene)-8a-methyl-3,4,8,8a-tetrahydronaphthalene-1,6(2H,7H)-dione-1-ethylene ketal as an orange oil. LCMS=251; (M+1)⁺. This material was used without further purification.

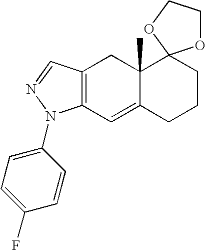

### Step C: (4aS)-1-(4-fluorophenyl)-4a-methyl-1,4,4a.6,7,8-hexahydro-5H-benzo[f]indazol-5-one-5-ethylene ketal

(7E,8aS)-7-(hydroxymethylene)-8a-methyl-3,4,8,8a-tetrahydronaphthalene-1,6(2H,7H)-dione-1-ethylene ketal (4.1 g, 16.4 mmol) was dissolved in glacial acetic acid (40 mL) and p-fluorophenylhyradzine hydrochloride (2.8 g, 17.22 mmol) and sodium acetate (1.41 g, 17.22 mmol) were added. After stirring at room temperature for 2 hours, the reaction was poured slowly into 10% NaHCO₃ (1 L) and extracted with EtOAc (6×500 mL). The combined extracts were washed with brine (500 mL), dried over MgSO₄ and concentrated in vacuo. The crude material was purified by flash chromatography (10% EtOAc/hexanes) on silica gel to afford 2.26 g (41%) of (4aS)-1-(4-fluorophenyl)-4a-methyl-1,4,4a,6,7,8-hexahydro-5H-benzo[f]indazol-5-one-5-ethylene ketal as an orange solid. LCMS=341; (M+1)⁺. ¹H NMR (CDCl₃, 500 MHz): δ 7.47-7.44 (m, 2H), 7.43 (s, 1H), 7.18-7.16 (d, J=8.5 Hz, 1H), 7.16-7.14 (d, J=8.7 Hz, 1H), 6.22 (br d, J=2.2 Hz, 1H), 4.11-4.01 (m, 4H), 3.20-3.16 (d, J=15.7 Hz, 1H), 2.54-2.51 (d, J=16 Hz, 1H), 2.51-2.40 (m, 1H), 2.34-2.28 (m, 1H), 1.88-1.64 (m, 4H), 1.23 (s, 3H).

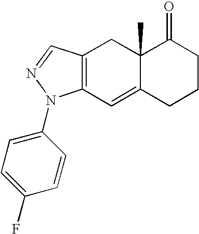

### Step D: (4aS)-1-(4-fluorophenyl)-4a-methyl-1,4,4a,6,7,8-hexahydro-5H-benzo[f]indazol-5-one

The (4aS)-1-(4-fluorophenyl)-4a-methyl-1,4,4a,6,7,8-hexahydro-5H-benzo[f]indazol-5-one-5-ethylene ketal (2.26 g; 6.65 mmol) was dissolved in THF (65 mL) and 6N HCl (4.43 mL, 26.6 mL) was added. The reaction was heated to 65° C. for 3.5 hours and then poured slowly into 10% NaHCO₃ (150 mL). The mixture was extracted with EtOAc (4×250 mL) and the combined extracts washed with brine (2×200 mL), dried over MgSO₄ and concentrated in vacuo to afford 1.97 g (100%) of (4aS)-1-(4-fluorophenyl)-4a-methyl-1,4,4a,6,7,8-hexahydro-5H-benzo[f]indazol-5-one as a brown oil. LCMS=297; (M+1)⁺. ¹H NMR (CDCl₃, 500 MHz): δ 7.50 (s, 1H), 7.49-7.45 (m, 2H), 7.20-7.16 (m, 2H), 6.31 (br d, J=2 Hz, 1 H), 2.96-2.88 (m, 2H), 2.72-2.62 (m, 2H), 2.59-2.53 (m, 2H), 2.14-2.80 (m, 1H), 1.75-1,64 (qt, J=13.1 Hz, J=4.3 Hz, 1H), 1.27 (s, 3H).

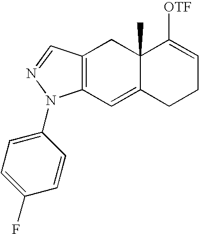

### Step E: (4aS)-1-(4-fluorophenyl)-4a-methyl-4,4a,7,8-tetrahydro-1H-benzo[f]indazol-5-yl trifluoromethanesulfonate

(4aS)-1-(4-fluorophenyl)-4a-methyl-1,4,4a,6,7,8-hexahydro-5H-benzo[f]indazol-5-one (0.44 g, 1.49 mmol) was dissolved in THF (30 mL) and cooled to 0° C. Potassium hexamethyldisilazide (40.51 mL of a 0.5 M solution in toluene, 20.28 mmol) was added. The reaction was allowed to warm to room temperature and stirred for 1.5 hours prior to addition of 2-[N,N-bis(trifluoromethylsulfonyl)amino]-5-chloropyridine (7.96 g, 20.28 mmol). The reaction was stirred at room temperature for 18 hours and then quenched with sat. NH₄Cl (50 mL) and H₂O (50 mL). The resultant mixture was extracted with EtOAc (4×200 mL) and the combined extracts were washed with H₂O (200 mL) and brine (200 mL), dried over MgSO₄ and concentrated in vacuo. The residue was filtered through a plug of silica and then purified by flash chromatography (5 to 15% EtOAC/hexanes) on silica gel to afford 1 g (46%) of (4aS)-1-(4-fluorophenyl)4a-methyl-4,4a,7,8-tetrahydro-1H-benzo[f]indazol-5-yl trifluoromethanesulfonate as a yellow solid. LCMS=429; (M+1)⁺. ¹H NMR (CDCl₃, 500 MHz): δ 7.51 (s, 1H), 7.50-7.46 (m, 2H), 7.21-7.19 (m, 2H), 6.33 (br d, J=1.6 Hz, 1H), 5.93-5.91 (dd, J=6.3 Hz, J=1.9 Hz, 1H), 2.97-2.93 (d, J=15.3 Hz, 1H), 2.77-2.73 (d, J=15.3 Hz, 1H), 2.54-2.38 (m, 3H), 2.34-2.25 (m, 1H), 1.26 (s, 3H).

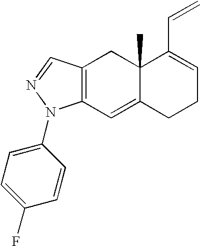

### Step F: (4aS)-1-(4-fluorophenyl)-4a-methyl-5-vinyl-4,4a,7,8-tetrahydro-1H-benzo[f]indazole

(4aS)-1-(4-fluorophenyl)-4a-methyl-4,4a,7,8-tetrahydro-1H-benzo[f]indazol-5-yl trifluoromethanesulfonate (1 g, 2.34 mmol) was dissolved in THF (25 mL) and LiCl (298 mg, 7.02 mmol) and tetrakis(triphenylphosphine) palladium (0) (54 mg, 0.047 mmol) were added followed by tributyl(vinyl)tin (716 μL, 2.45 mmol). The reaction was refluxed under N₂ for 18 hours and then concentrated in vacuo. The residue was partitioned between EtOAc (50 mL) and 10% NH₄OH (50 mL). The aqueous layer was extracted with EtOAc (50 mL) and the combined organic layers were washed with H₂O (50 mL) and brine (50 mL), dried over MgSO₄ and concentrated in vacuo. The product was purified by flash chromatography (5% EtOAc/hexanes) to afford 650 mg (91%) of (4aS)-1-(4-fluorophenyl)-4a-methyl-5-vinyl-4,4a,7,8-tetrahydro-1H-benzo[f]indazole as an off-white solid. LCMS=307; (M+1)⁺. ¹H NMR (CDCl₃, 500 MHz): δ 7.51-7.47 (m, 2H), 7.45 (s, 1H), 7.18-7.17 (d, J=8.4 Hz, 1H), 7.17-7.16 (d, J=8.6 Hz, 1H), 6.40-6.38 (d, J=17.1 Hz, J=10.8 Hz, 1H), 6.24 (br d, J=2 Hz, 1H), 5.99-5.97 (d, J=5.3 Hz, 1H), 5.44-5.40 (dd, J=17.1 Hz, J=1.6 Hz, 1H), 5.08-5.06 (dd, J=10.9 Hz, J=1.8 Hz, 1H), 2.92-2.89 (d, J=15.5 Hz, 1H), 2.54-2.51 (d, J=15.5 Hz, 1H), 2.48-2.42 (m, 1H), 2.39-2.29 (m, 2H), 2.20-2.13 (m, 1H), 1.16 (s, 3H).

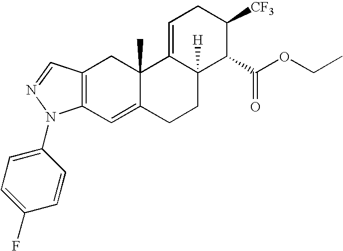

### Step G: ethyl (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylate

(4aS)-1-(4-fluorophenyl)-4a-methyl-5-vinyl-4,4a,7,8-tetrahydro-1H-benzo[f]indazole (710 mg, 2.32 mmol) was dissolved in CH₂Cl₂ (30 mL). Ethyl-4,4,4-trifluorocrotonate (381 μL; 2.55 mmol) was added, followed by dropwise addition of BCl₃ (5.8 mL of a 1.0 M solution in CH₂Cl₂, 5.8 mmol). The reaction was stirred at room temperature for 3 hours and then quenched with sat. NH₄Cl. The reaction was partitioned between CH₂Cl₂ (50 mL) and H₂O (50 mL) and the aqueous layer was extracted with CH₂Cl₂ (2×50 mL). The combined organic layers were washed with 1N HCl (50 mL) and brine (50 mL), dried over MgSO₄ and concentrated in vacuo. The residue was purified by flash chromatography (0 to 65% EtOAc/hexanes) on silica gel to afford 920 mg (84%) of the product as a mixture of 2 diastereomers. Separation by chiral HPLC (ChiralPak AD column; 15% isopropanol/heptane) gave 562 mg (61%) of ethyl (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylate as a colorless solid. LCMS=475; (M+1)⁺. ¹H NMR (CDCl₃, 500 MHz): δ 7.49-7.45 (m, 2H), 7.43 (s, 1H), 7.19-7.17 (d, J=8.5 Hz, 1H), 7.17-7.15 (d, J=8.4 Hz, 1H), 6.21 (br s, 1 H), 5.75 (br d, J=5.9 Hz, 1H), 4.22-4.18 (q, J=7.1 Hz, 2H), 2.90-2.86 (d, J=15.8 Hz, 1H), 2.81-2.74 (m, 1H), 2.69-2.60 (m, 1H), 2.53-2.47 (m, 2H), 2.44-2.22 (m, 4H), 1.97-1.89 (m, 1H), 1.62-1.54 (m, 1H), 1.30-1.27 (t, J=7.1 Hz, 3H), 1.24 (s, 3H).

### EXAMPLE 2

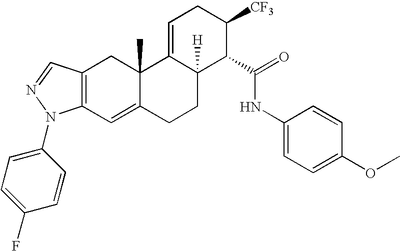

### (3R,4R,4aR,11aS)-8-(4-fluorophenyl)-N-(4-methoxyphenyl)-11a-methyl-3-(trifluoromethyl)-3,4,4a,5,6,8,11,11a-octahydro-2H-naphtho[1,2-f]indazole-4-carboxamide

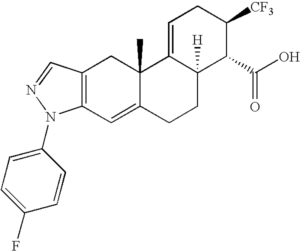

### Step A: (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid

Ethyl (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylate (106 mg, 0.224 mmol) was dissolved in 1:1:1 THF:MeOH:H₂O (15 mL) and LiOH.H₂O (94 mg, 2.24 mmol) was added. The reaction was stirred at room temperature for 60 hours and then concentrated in vacuo. The residue was dissolved in 1N NaOH (15 mL) and washed with diethyl ether (15 mL). The aqueous layer was acidified to pH=3 with 1N HCl and extracted with EtOAc (4×25 mL). The combined extracts were dried over Na₂SO₄ and concentrated in vacuo to afford 99 mg (99%) of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid as an off-white solid. LCMS=447; (M+1)⁺. ¹H NMR (CDCl₃, 500 MHz): δ 7.49-7.46 (m, 2H), 7.44 (s, 1H), 7.20-7.16 (m, 2H), 6.23 (br s, 1H), 5.79-5.77 (br d, J=5.7 Hz, 1H), 2.90-2.85 (d, J=15.8 Hz, 1H), 2.83-2.76 (m, 1H), 2.74-2.66 (m, 1H), 2.58-2.53 (dd, J=11.1 Hz, J=9.1 Hz, 1H) 2.54-2.51 (d, J=15.7 Hz, 1H), 2.48-2.24 (m, 4H), 2.07-1.99 (m, 1H), 1.67-1.59 (m, 1H), 1.25 (s, 3H).

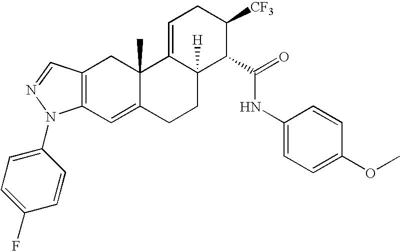

### Step B: (3R,4R,4aR,11aS)-8-(4-fluorophenyl)-N-(4-methoxyphenyl)-11a-methyl-3-(trifluoromethyl)-3,4,4a,5,6,8,11,11a-octahydro-2H-naphtho[1,2-f]indazole-4-carboxamide

(3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid (7 mg, 0.0157 mmol) was dissolved in CH₂Cl₂ (2 mL) and O-(7-azabenzotriazol-1-yl)-N,N,N′,N′-tetramethyluronium hexafluorophosphate (12 mg, 0.0314 mmol), N,N-diisopropylethylamine (42.6 mL, 0.2355 mmol) and p-anisidine (19.3 mg, 0.157 mmol) were added. The reaction was stirred at room temperature for 18 hours. The reaction was diluted with H₂O (5 mL) and shaken. The H₂O was decanted off; the organic layer was dried over MgSO₄ and concentrated in vacuo. The residue was purified by flash chromatography (0 to 25% EtOAc/hexanes) on silica gel to afford 7.7 mg (89%) of (3R,4R,4aR,11aS)-8-(4-fluorophenyl)-N-(4-methoxyphenyl)-11a-methyl-3-(trifluoromethyl)-3,4,4a,5,6,8,11,11a-octahydro-2H-naphtho[1,2-f]indazole-4-carboxamide as an off-white solid. LCMS=552 (M+1)⁺. ¹H NMR (CDCl₃, 500 MHz): δ 7.49-7.45 (m, 2H), 7.43 (s, 1H), 7.42-7.40 (m, 2H), 7.19-7.15 (m, 3H), 6.90-6.87 (m, 2H), 6.20 (br s, 1H), 5.78-5.76 (br d, J=6.2 Hz, 1H), 3.81 (s, 3H), 2.99-2.93 (m, 1H), 2.93-2.89 (d, J=15.7 Hz, 1H), 2.81-2.72 (m, 1H), 2.57-2.53 (d, J=15.8 Hz, 1H), 2.30-2.25 (m, 4H), 2.25-2.20 (dd, J=11.2 Hz, J=9.4 Hz, 1H), 2.13-2.05 (m, 1H), 1.25 (s, 3H).

The following compounds were synthesized by procedures analogous to that described in EXAMPLE 2:

| Compound | Molecular structure | LCMS (M + 1)⁺ |
|---|---|---|
| 3 | 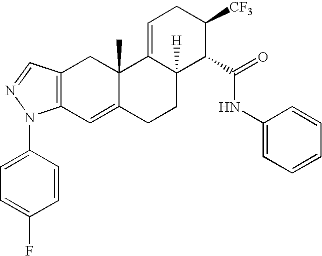 | 522 |
| 4 | 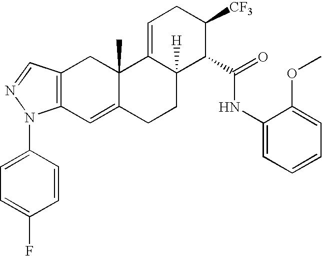 | 552 |
| 5 | 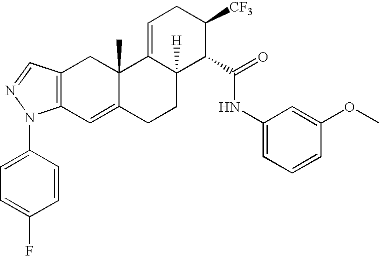 | 552 |
| 6 | 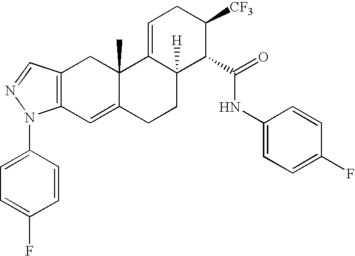 | 540 |
| 7 | 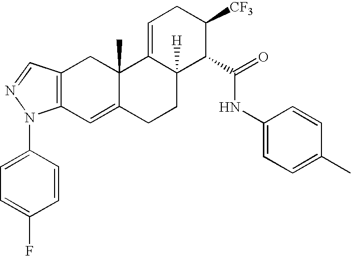 | 536 |
| 8 | 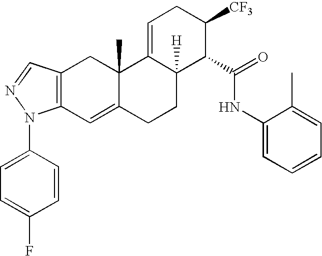 | 536 |
| 9 | 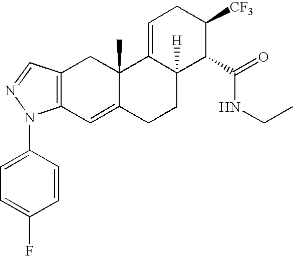 | 474 |
| 10 | 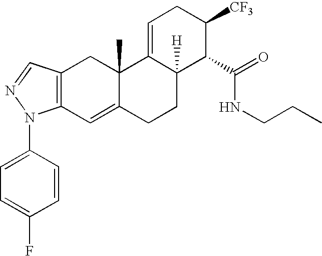 | 488 |
| 11 | 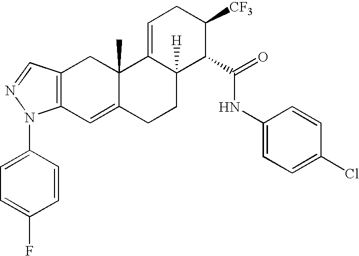 | 557 |
| 12 | 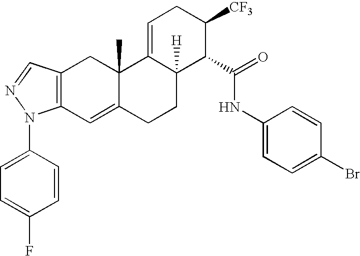 | 601 |
| 13 | 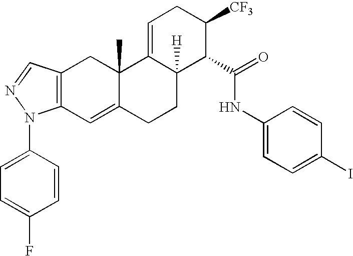 | 648 |
| 14 | 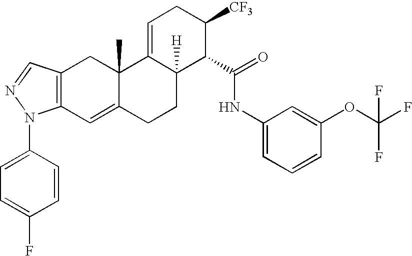 | 606 |
| 15 | 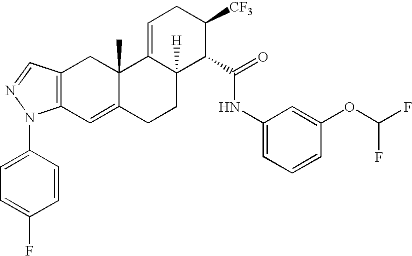 | 588 |
| 16 | 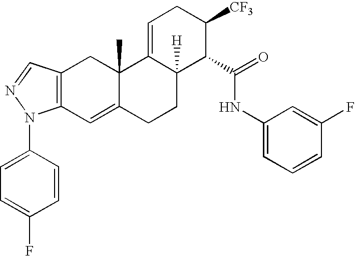 | 540 |
| 17 | 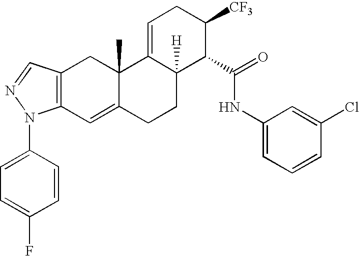 | 557 |
| 18 | 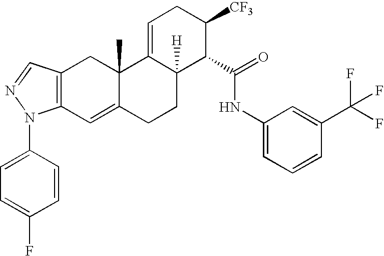 | 590 |
| 19 | 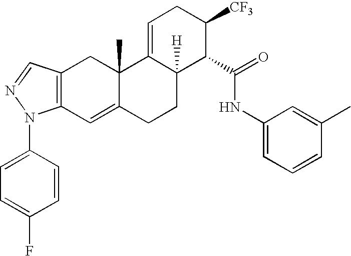 | 536 |
| 20 | 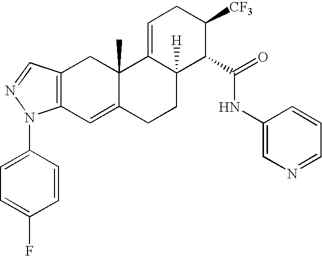 | 523 |
| 21 | 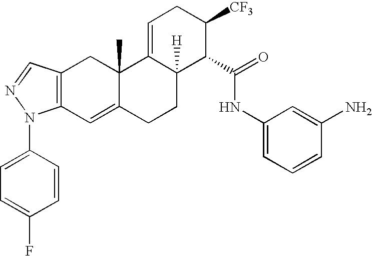 | 537 |
| 22 | 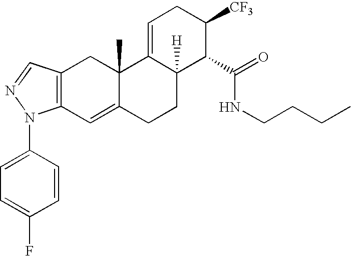 | 502 |
| 23 | 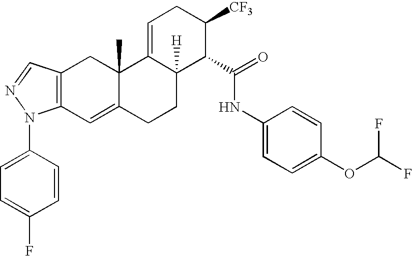 | 588 |
| 24 | 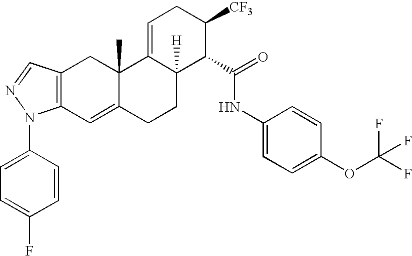 | 606 |
| 25 | 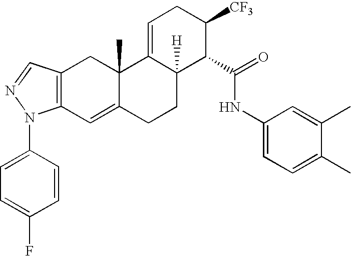 | 550 |
| 26 | 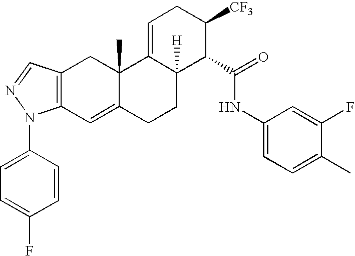 | 554 |
| 27 | 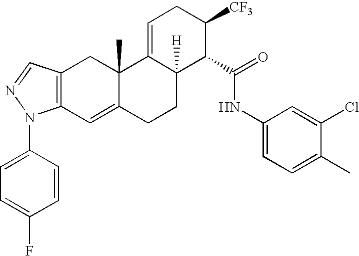 | 571 |
| 28 | 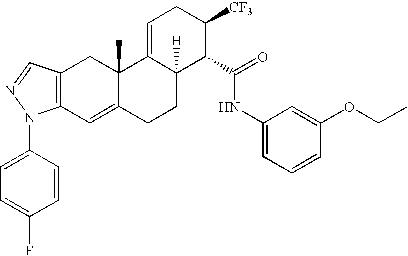 | 566 |
| 29 | 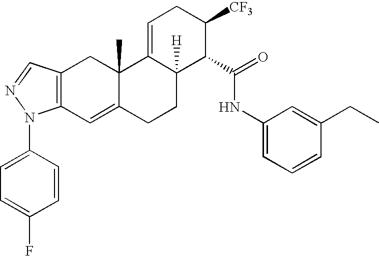 | 550 |
| 30 | 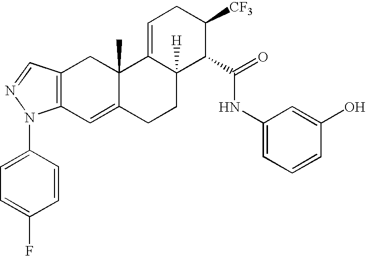 | 538 |
| 31 | 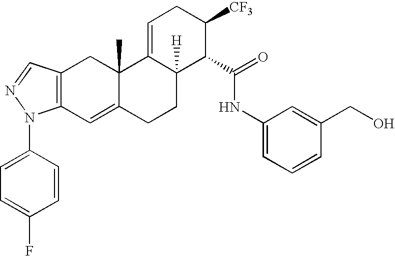 | 552 |
| 32 | 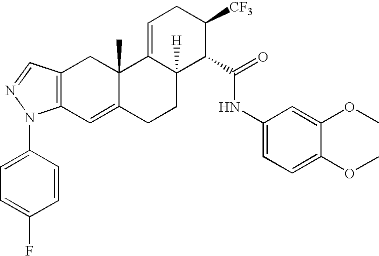 | 582 |
| 33 | 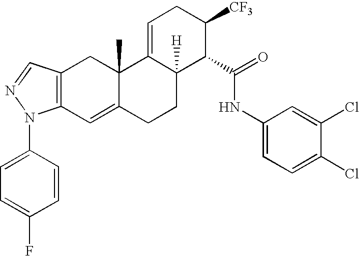 | 591 |
| 34 | 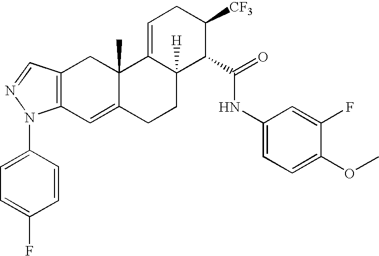 | 570 |
| 35 | 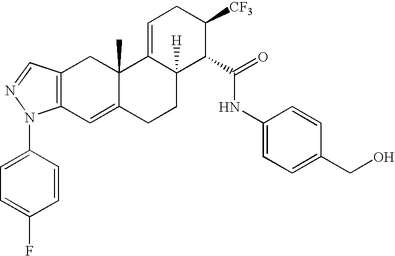 | 552 |
| 36 | 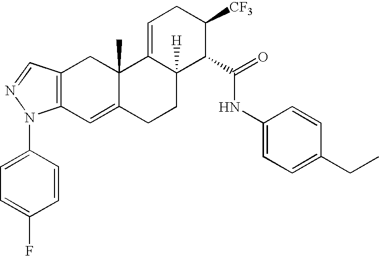 | 550 |
| 37 | 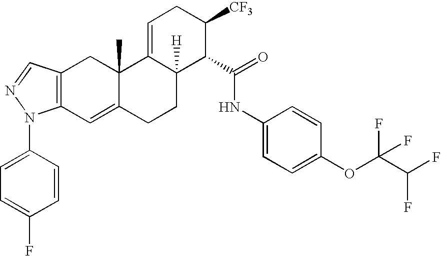 | 638 |
| 38 | 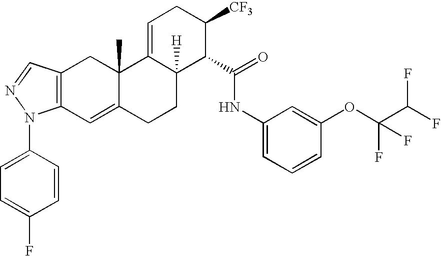 | 638 |
| 39 | 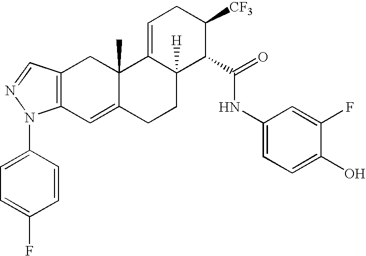 | 556 |
| 106 | 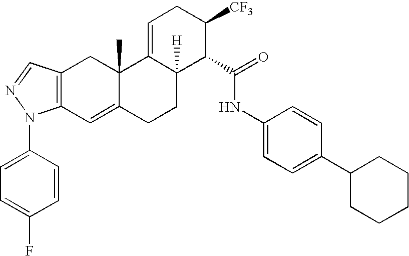 | 604 |
| 107 | 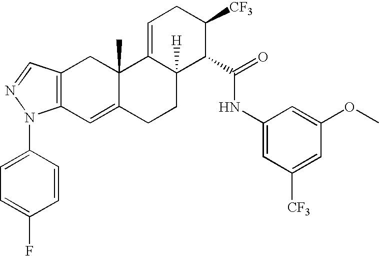 | 620 |
| 108 | 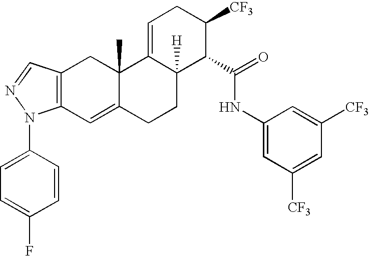 | 658 |

### EXAMPLE 40

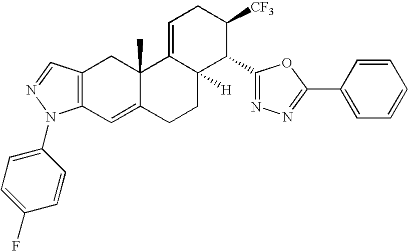

### (3R,4R,4aR,11aS)-8-(4-fluorophenyl)-11a-methyl-4-(5-phenyl-1,3,4-oxadiazol-2-yl)-3-(trifluoromethyl)-3,4,4a,5,6,8,11,11a-octahydro-2H-naphtho[1,2-f]indazole

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid (90 mg, 0.2 mmol) prepared as in EXAMPLE 2 Step A in POCl₃ (2 mL) was added benzoic hydrazide (55 mg, 0.4 mmol), and the mixture was heated at 100° C. for 18 h. The reaction mixture was cooled and poured into ice. It was made basic (˜pH 8) with conc. NH₄OH while cooling the mixture in ice/H₂O bath. Brine was added to the mixture and it was extracted with CH₂Cl₂ three times. After drying over Na₂SO₄, it was filtered and concentrated under reduced pressure. The crude material was purified by preparative TLC eluting with 8% EtOAc/CH₂Cl₂ to obtain 53.7 mg of the title compound.

¹H NMR (CDCl₃, 500MHz): δ 8.03 (d, J=6.9 Hz, 2H); 7.53-7.42 (m, 6H); 7.13 (t, J=8.6 Hz, 2H); 6.18 (d, J=1.4 Hz, 1H); 5.85 (d, J=6.0 Hz, 1H); 3.26 (dd, J=11, 9.8 Hz, 1H); 3.0 (m, 1H); 2.91 (d, J=15.8 Hz, 1H); 2.81 (m, 1H); 2.55 (d, J=15.8 Hz, 1H); 2.5 (m, 1H); 2.4 (m, 2H); 2.29 (m, 1H); 1.85 (m, 1H); 1.6 (m, 1H); 1.27 (s, 3H). Mass spectrum (ESI): 547.1 (M+1).

### EXAMPLE 41

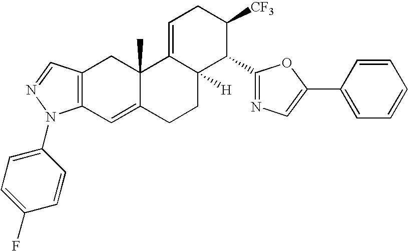

### (3R,4R,4aR,11aS)-8-(4-fluorophenyl)-11a-methyl4-(5-phenyl-1,3-oxazol-2-yl)-3-(trifluoromethyl)-3,4,4a,5,6,8,11,11a-octahydro-2H-naphtho[1,2-f]indazole

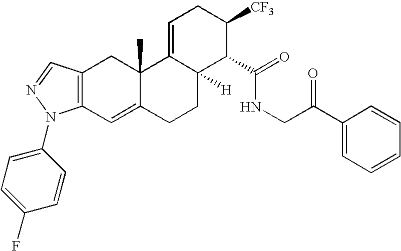

### Step A: (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-(2-oxo-2-phenylethyl)carboxamide

The solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid (40.8 mg, 0.091 mmol) in CH₂Cl₂ (1.0 mL) was cooled down to 0° C., and to this was added oxalyl chloride (70 μL, 2.0 M, 0.137 mmol) followed by 1 drop of DMF via pipet. The reaction mixture was stirred for 2 h while slowly warming up to room temperature. The solvent was removed under reduced pressure, and the residue was co-evaporated two times with toluene. After drying under high vacuum, the acid chloride was dissolved in CH₂Cl₂ (1.0 mL), and to this was added 2-aminoacetophenone hydrochloride (18 mg, 0.1 mmol) followed by Et₃N (28 μL, 0.2 mmol). The reaction mixture was stirred for 2 h, then 10% aqueous NH₄OH was added. The aqueous layer was extracted with CH₂_{Cl}₂, and combined extracts were washed with brine and dried over Na₂SO₄. The crude material was purified by preparative TLC eluting with 1:2 acetone:hexane to obtain 42 mg of the title compound.

¹H NMR (CDCl₃, 500MHz): δ 7.97 (d, J=7.6 Hz, 2H); 7.62-7.39 (m, 6H); 7.14 (t, J=8.6 Hz, 2H); 6.82 (1 NH); 6.16 (s, 1H); 5.73 (d, J=6.0 Hz, 1H); 4.91 (dd, J=5, 19.9 Hz, 1H); 4.68 (dd, J=3.5, 19.9 Hz, 1H); 2.86 (m, 1H); 2.87 (d, J=15.8 Hz, 1H); 2.71 (m, 1H); 2.5 (d, J=15.8 Hz, 1H); 2.44-2.21 (m, 5H); 2.01 (m, 1H); 1.5 (m, 1H); 1.22 (s, 3H). Mass spectrum (ESI): 564.2 (M+1).

### Step B: (3R,4R,4aR,11aS)-8-(4-fluorophenyl)-11a-methyl-4-(5-phenyl-1,3-oxazol-2-yl)-3-(trifluoromethyl)-3,4,4a,5,6,8,11,11a-octahydro-2H-naphtho[1,2-f]indazole

The solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-(2-oxo-2-phenylethyl)carboxamide (17.4 mg, 0.031 mmol) in POCl₃ (0.5 mL) was heated at 100° C. for 3 h and cooled to room temperature. The reaction mixture was poured into ice/H₂O and made basic with conc. NH₄OH to pH 8 while cooling in ice bath (very exothermic). Brine was added and the aqueous layer was extracted with CH₂Cl₂ three times. The combined organic layer was dried over Na₂SO₄, filtered and concentrated under reduced pressure. The crude material was purified by preparative TLC eluting with 1:3 acetone:hexane system to obtain 12.2 mg of the title compound.

¹H NMR (CD₃OD, 500 MHz): δ 7.65 (d, J=7.3 Hz, 2H); 7.45-7.39 (m, 6H); 7.32 (m, 1H); 7.22 (t, J=8.7 Hz, 2H); 6.18 (s, 1H); 5.89 (d, J=5.7 Hz, 1H); 3.08 (t, J=10.6 Hz, 1H); 2.97 (m, 1H); 2.9 (d, J=16 Hz, 1H); 2.84 (m, 1H); 2.6 (d, J=16 Hz, 1H); 2.49 (m, 1H); 2.38 (m, 2H); 2.28 (m, 1H); 1.7 (m, 1H); 1.56 (m, 1H); 1.24 (s, 3H). Mass spectrum (ESI): 546.2 (M+1).

The following examples were synthesized by pocedures analogous to that described in EXAMPLE 41:

| Compound | Molecular Structure | LCMS (M + 1)⁺ |
|---|---|---|
| 114 | 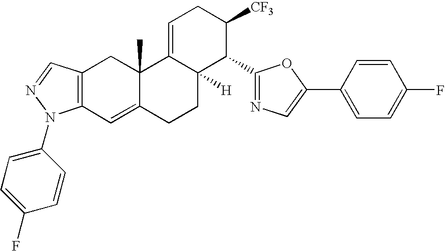 | 564 |
| 115 | 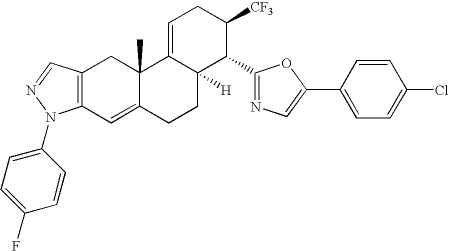 | 580 |
| 116 | 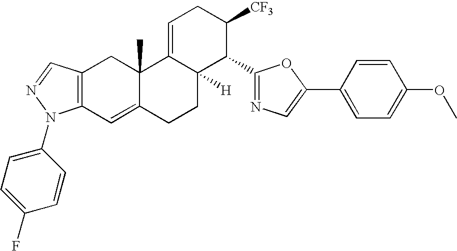 | 576 |
| 117 | 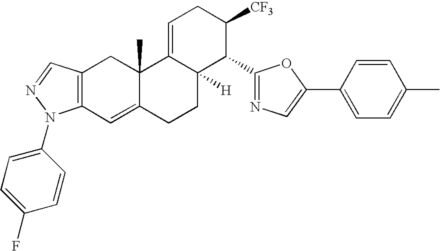 | 560 |

### EXAMPLE 42

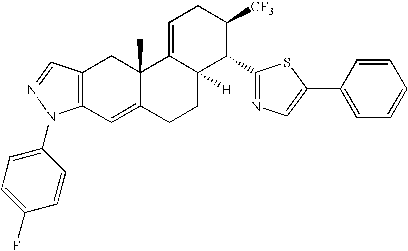

### (3R4R,4aR,11aS)-8-(4-fluorophenyl)-11a-methyl-4-(5-phenyl-1,3-thiazol-2-yl)-3-(trifluoromethyl)-3,4,4a,5,6,8,11,11a-octahydro-2H-naphtho[1,2-f]indazole

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-(2-oxo-2-phenylethyl)carboxamide (25.4 mg, 0.045 mmol) in toluene (1 mL) was added Lawesson's reagent (73 mg, 0.18 mmol), and the mixture was heated at 100° C. for 3 h. The reaction mixture was cooled and the solvent was removed under reduced pressure. The crude material was purified by preparative TLC eluting with 1:3 acetone:hexane system to give 18.4 mg of the title compound.

¹H NMR (CD₃OD, 500 MHz): δ 7.98 (s, 1H); 7.59 (d, J=7.6 Hz, 2H); 7.46-7.38 (m, 5H); 7.33 (t, J=7.6 Hz, 1H); 7.24 (t, J=8.7 Hz, 2H); 6.19 (s, 1H); 5.9 (d, J=6.0 Hz, 1H); 3.24 (dd, J=9.6, 11 Hz, 1H); 2.94 (m, 1H); 2.94 (d, J=15.9 Hz, 1H); 2.83 (m, 1H); 2.6 (d, J=15.9 Hz, 1H); 2.5 (m, 1); 2.38 (m, 2H); 2.29 (m, 1H); 1.74 (m, 1H); 1.59 (m, 1H); 1.26 (s, 3H). Mass spectrum (ESI): 562.2 (M+1).

### EXAMPLE 43

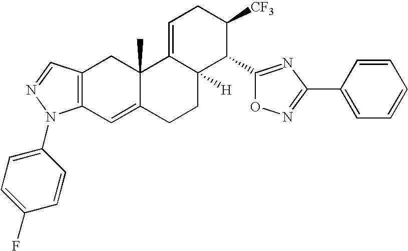

### (3R,4R,4aR,11aS)-8-(4-fluorophenyl)-11a-methyl4-(3-phenyl-1,2,4-oxadiazol-5-yl)-3-(trifluoromethyl)-3,4,4a,5,6,8,11,11a-octahydro-2H-naphtho[1,2-f]indazole

A solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid (16.7 mg, 0.037 mmol) in CH₂Cl₂ (0.5 mL) was cooled down to 0° C., and to this was added oxalyl chloride (28 μL, 2.0 M, 0.056 mmol) followed by 1 drop of DMF via pipet. The reaction mixture was stirred for 2 h while slowly warming up to room temperature. The solvent was removed under reduced pressure, and the residue was co-evaporated two times with toluene. After drying under high vacuum, the acid chloride was dissolved in THF (0.5 mL) and benzamide oxime (16 mg., 0.12 mmol) was added and the mixture was stirred at 65° C. for 3 hours. The mixture was cooled to room temperature and Bu₄NF (40 μL of a 1.0 M solution in THF) was added. The reaction was stirred overnight. The mixture was heated to and maintained at 65° C. for 6 h, and then stirred a room temperature overnight. More Bu₄NF (40 μL of a 1.0 M solution in THF) was added and the reaction was heated to 65° C. for 2 h. The mixture was cooled and was diluted with EtOAc. The organic layer was washed with H₂O and brine, then dried over Na₂SO₄. The crude material was purified by preparative TLC eluting with 1:3 acetone:hexane system to obtain 14.1 mg of the title compound.

¹H NMR (CDCl₃, 500MHz): δ 8.08 (dd, J=1.6, 8.0 Hz, 2H); 7.49-7.42 (m, 6H); 7.14 (t, J=8.6 Hz, 2H); 6.19 (s, 1H); 5.85 (d, J=5.7 Hz, 1H); 3.27 (dd, J=9.6, 11.2 Hz, 1H); 3.04 (m, 1H); 2.93 (d, J=15.8 Hz, 1H); 2.56 (d, J=15.8 Hz, 1H); 2.51 (m, 1H); 2.39 (m, 2H); 2.29 (m, 1H); 1.78 (m, 1H); 1.58 (m, 1H); 1.27 (s, 3H). Mass spectrum (ESI): 547.2 (M+1).

### EXAMPLE 44

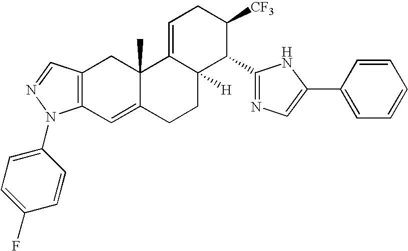

### (3R,4R,4aR,11aS)-8-(4-fluorophenyl)-11a-methyl-4-(5-phenyl-1H-imidazol-2-yl)-3-(trifluoromethyl)-3,4,4a,5,6,8,11,11a-octahydro-2H-naphtho[1,2-f]indazole

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (EXAMPLE 47) (12 mg, 0.028 mmol) in methanol (0.5 mL) was added phenyl glyoxal hydrate (11 mg, 0.084 mmol) and ammonium acetate (22 mg, 0.28 mmol). The mixture was heated at 65° C. for 4 h. The reaction mixture was cooled down to room temperature and removed methanol under reduced pressure. The residue was dissolved in CH₂Cl₂ and washed with saturated NaHCO₃, H₂O, and brine, respectively. The organic layer was dried over Na₂SO₄, then the crude material was purified by preparative TLC eluting with 1:2 EtOAc:hexane to give 5 mg of the title compound.

¹H NMR (CD₃OD, 500 MHz): δ 7.63 (d, J=7.5 Hz, 2H); 7.45-7.39 (m, 3H); 7.33 (t, J=7.7 Hz, 2H); 7.29 (s, 1H); 7.24-7.18 (m, 3H); 6.15 (s, 1H); 5.88 (d, J=6.0 Hz, 1H); 2.95 (d, J=16.2 Hz, 1H); 2.93 (m, 2H); 2.83 (m, 1H); 2.59 (d, J=16.2 Hz, 1H); 2.48 (m, 1H); 2.36 (m, 2H); 2.27 (m, 1H); 1.64 (m, 1H); 1.50 (m, 1H); 1.21 (s, 3H). Mass spectrum (ESI): 545.2 (M+1).

### EXAMPLE 45

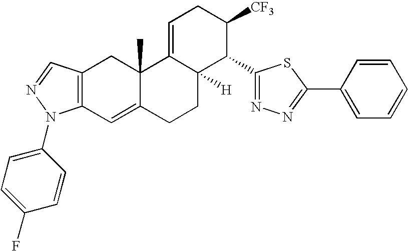

### 3R,4R,4aR,11aS)-8-(4-fluorophenyl)-11a-methyl-4-(5-phenyl-1,3,4-thiadiazol-2-yl)-3-(trifluoromethyl)-3,4,4a,5,6,8,11,11a-octahydro-2H-naphtho[1,2-f]indazole

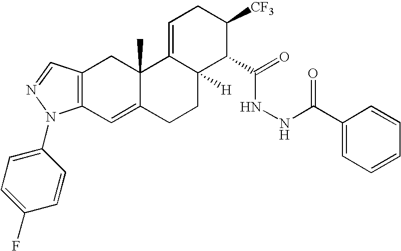

### Step A: (3R,4R,4aR,11aS)-N′-benzoyl-8-(4-fluorophenyl)-11a-methyl-3-(trifluoromethyl)-3,4,4a,5,6,8,11,11a-octahydro-2H-naphtho[1,2-f]indazole-4-carbohydrazide

The title compound was prepared according to the procedure described in EXAMPLE 41 Step A using benzoic hydrazide.

¹H NMR (CDCl₃, 500 MHz): δ 10.1 (d, J=5.5 Hz, 1H); 9.89 (d, J=5.5 Hz, 1H); 7.8 (d, J=7.1 Hz, 2); 7.49-7.35 (m, 6H); 7.14 (t, J=8.6 Hz, 2H); 6.12 (s, 1H); 5.65 (d, J=6.0 Hz, 1H); 2.79 (d, J=15.8 Hz, 11); 2.78 (m, 1H); 2.63 (m, 1H); 2.55 (dd, J=9.4, 11.0 Hz, 1H); 2.46 (d, J=15.8 Hz, 1H); 2.35 (m, 1H); 2.31 (m, 1H); 2.24 (m, 2H); 2.09 (m, 1H); 2.0 (m, 1H); 1.54 (m, 1H); 1.15 (s, 3H).

### Step B: (3R,4R,4aR,11aS)-8-(4-fluorophenyl)-11a-methyl4-(5-phenyl-1,3,4-thiadiazol-2-yl)-3-(trifluoromethyl)-3,4,4a,5,6,8,11,11a-octahydro-2H-naphtho[1,2-f]indazole

The title compound was prepared according to the procedure described in EXAMPLE 42 starting from (3R,4R,4aR,11aS)-N′-benzoyl-8-(4-fluorophenyl)-11a-methyl-3-(trifluoromethyl)-3,4,4a,5,6,8,11,11a-octahydro-2H-naphtho[1,2-f]indazole-4-carbohydrazide.

¹H NMR (CDCl₃, 500 MHz): δ 7.94 (dd, J=7.6, 2.0 Hz, 2H); 7.48-7.42 (m, 6H); 7.14 (t, J=8.6 Hz, 2H); 6.17 (d, J=1.4 Hz, 1H); 5.85 (d, J=5.8 Hz, 1H); 3.45 (dd, J=10.4, 9.0 Hz, 1H); 2.96 (m, 1H); 2.92 (d, J=15.8 Hz, 1H); 2.84 (m, 1H); 2.54 (d, J=15.8 Hz, 1H); 2.52 (m, 1H); 2.44 (m, 1H); 2.38 (m, 1H); 2.35 (m, 1H); 2.26 (m, 1H); 1.82 (m, 1H); 1.62 (m, 1H); 1.26 (s, 3H). Mass spectrum (ESI): 563.2 (M+1).

### EXAMPLE 46

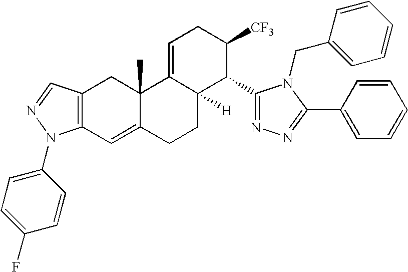

### (3R,4R,4aR,11aS)-4-(4-benzyl-5-phenyl-4H-1,2,4-triazol-3-yl)-8-(4-fluorophenyl)-11a-methyl-3-(trifluoromethyl)-3,4,4a,5,6,8,11,11a-octahydro-2H-naphtho[1,2-f]indazole

(3R,4R,4aR,11aS)-8-(4-fluorophenyl)-11a-methyl-4-(3-phenyl-1,2,4-oxadiazol-5-yl)-3-(trifluoromethyl)-3,4,4a,5,6,8,11,11a-octahydro-2H-naphtho[1,2-f]indazole (EXAMPLE 43) (31.6 mg, 0.058 mmol) was dissolved in benzylamine (1 mL) in a sealed tube, and the mixture was heated at 150° C. for 13 days. It was cooled, loaded onto packed silica gel, and eluted with diethyl ether (50 mL) to remove benzyl amine followed by 5% MeOH/CH₂Cl₂ to obtain the crude material. Further purification was done by a preparative TLC eluting with 3% MeOH/CH₂Cl₂ to obtain 3.6 mg of the title compound.

¹H NMR (CDCl₃, 500 MHz): δ 7.61-6.99 (15 aromatic H's); 6.07 (s, 1H); 5.75 (d, J=5.9 Hz, 1H); 5.34 (d, J=16.7 Hz, 1H); 5.07 (d, J=16.7 Hz, 1H); 3.21 (m, 1H); 2.88 (d, J=15.7 Hz, 1H); 2.82 (m, 1H); 2.75 (dd, J=10.5, 8.9 Hz, 1H); 2.52 (m, 1H); 2.49 (d, J=15.7 Hz, 1H); 2.33 (m, 1H); 2.06 (m, 2H); 1.38 (m, 1H); 1.14 (s, 3H); 0.55 (m, 1H). Mass spectrum (ESI): 636.4 (M+1).

### EXAMPLES 47 and 48

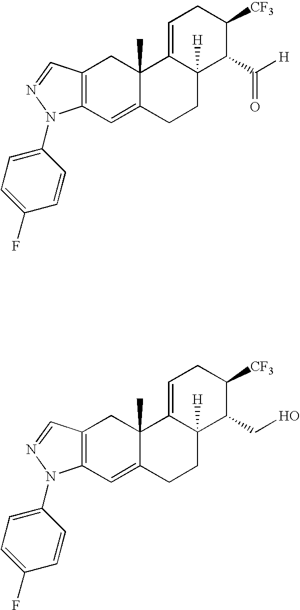

### (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde and [(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanol

To a solution of ethyl (11aS)-8-(4-fluorophenyl)-11a-methyl-3-(trifluoromethyl)-3,4,4a,5,6,8,11,11a-octahydro-2H-naphtho[1,2-f]indazole-4-carboxylate (from EXAMPLE 1) (505 mg, 1.064 mmol) in CH₂Cl₂ (15 mL) was added diisobutylaluminum hydride (2.13 mL of a 1M solution in CH₂Cl₂, 2.13 mmol) at −78° C. The mixture stirred for 2 hours at −78° C. and was quenched with Rochelle's salt. The organic layer was extracted with water, brine, and dried over MgSO₄. The crude product was purified by liquid chromatography, 5% to 100% ethyl acetate in hexanes to yield (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (300 mg, 67 %) and [(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanol (108 mg, 24%).

EXAMPLE 47: (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde

¹H NMR (CDCl₃, 500 MHz): δ 9.607 (s, 1H), 7.458 (m, 2H), 7.408 (s, 1H), 7.164 (m, 2H), 6.226 (s, 1H), 5.764 (s, 1H), 2.854 (d, J=15.8 Hz, 1H), 2.767-2.692 (m, 2H), 2.531-2.518 (m, 1H), 2.484 (d, 15.8 Hz, 1H), 2.410-2.039 (m, 4H), 1.927 (m, 1H), 1.602 (m, 1H), 1.233 (s, 3H).

EXAMPLE 48: [(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanol

¹H NMR (CDCl₃, 500 MHz): δ 7.451 (m, 2H), 7.395 (s, 1H), 7.146 (t, J=8.7 Hz, 2H), 6.190 (s, 1H), 5.716 (s, 1H), 3.845 (dd, J=3.2, 11.7 Hz, 1H), 3.660 (dd, J=3.2, 11.7 Hz, 1H), 2.813 (d, J=15.6 Hz, 1H), 2.551 (m, 1H), 2.470 (m, 1H), 2.398-2.246 (m, 5H), 2.057 (m, 1H), 1.757 (m, 1H), 1.520 (m, 1H), 1.210 (s, 3H).

### EXAMPLE 49

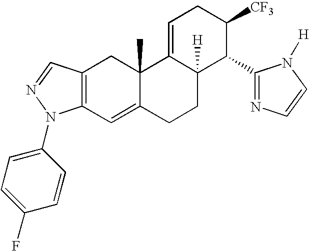

### (3R,4R,4aR,11aS)-8-(4-fluorophenyl)-4-(1H-imidazol-2-yl)-11a-methyl-3-(trifluoromethyl)-3,4,4a,5,6,8,11,11a-octahydro-2H-naphtho[1,2-f]indazole

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (EXAMPLE 47) (144 mg, 0.334 mmol) in methanol (6 mL) was added glyoxal (58.4 mg, 0.998 mmol) and ammonium acetate (528 mg, 6.68 mmol) at room temperature. The reaction mixture was heated to reflux for 4 days, then the solvent was removed under reduced pressure. The crude mixture was diluted with ethyl acetate and washed with NaHCO₃, H₂O, brine, and dried over MgSO₄. After removal of the solvent, the crude product was purified twice by preparative thin layer chromatography eluting with 4% methanol in dichloromethane and then once by flash chromatography on silica gel using a gradient of 5% acetone in hexanes increasing to 100% acetone to give 54 mg of the title compound.

¹H NMR (CDCl₃, 500 MHz): δ 7.471 (m, 3H), 7.297 (t, J=8.7 Hz, 2H), 6.209 (s, 1H), 5.905 (d, J=6.0 Hz, 1H), 2.953 (t, J=15.8 Hz, 1H), 2.915 (m, 2H), 2.752 (m, 1H), 2.643 (d, J=15.8 Hz, 1H), 2.273-2.518 (m, 4H), 1.601 (m, 1H), 1.497 (m, 1H), 1.278 (s, 3H).

### EXAMPLE 50

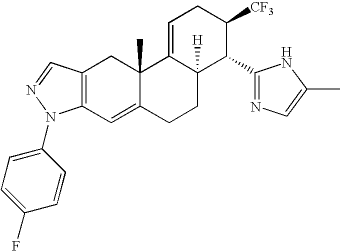

### (3R,4R,4aR,11aS)-8-(4-fluorophenyl)-4-(1H-imidazol-2-yl)-11a-methyl-3-(trifluoromethyl)-3,4,4a,5,6,8,11,11a-octahydro-2H-naphtho[1,2-f]indazole

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (EXAMPLE 47) and pyruvic aldehyde according to the procedure described in EXAMPLE 49

¹H NMR (CDCl₃, 500 MHz): δ 7.476 (m, 31), 7.297 (m, 3H), 6.269 (s, 1H), 6.011 (d, J=6.8 Hz, 1H), 3.194 (t, J=11.8 Hz, 1H), 2.940 (d, J=17.8 Hz, 1H), 2.890 (m, 2H), 2.689 (d, J=17.8 Hz, 1H), 2.657 (m, 1H), 2.477-2.39 (m, 6H), 1.692 (m, 1H), 1.535 (m, 1H), 1.313 (s, 3H).

### EXAMPLE 51

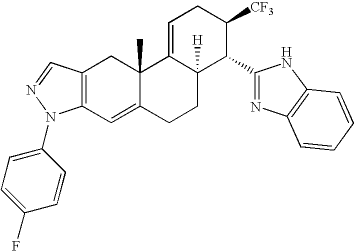

### (3R,4R,4aR,11aS)-4-(1H-benzimidazol-2-yl)-8-(4-fluorophenyl)-11a-methyl-3-(trifluoromethyl)-3,4,4a,5,6,8,11,11a-octahydro-2H-naphtho[1,2-f]indazole

(3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (EXAMPLE 47) (15 mg, 0.035 mmol) and 1,2-phenylenediamine (10 mg) in nitrobenzene (0.4 mL) were heated to 150° C. for 4 hours. The nitrobenzene was evaporated and the crude residue was purified by preparative thin layer chromatography (1:1 EtOAc:hexane) to yield 3.2 mg of the title compound.

¹H NMR (CDCl₃, 500 MHz): δ 7.575 (bs, 1H), 7.473 (m, 3H), 7.410 (s, 1H), 7.249 (m, 2H), 7.166 (t, J=8.7 Hz, 2H), 6.153 (s, 1H), 5.814 (d, J=5.9 Hz, 1H), 3.246 (m, 1H), 3.030 (t, J=11.0 Hz, 1H), 2.853 (m, 2H), 2.521 (m, 2H), 2.383 (m, 2H), 2.258 (m, 2H), 1.790 (m, 1H), 1.504 (m, 1H), 1.275 (s, 3H).

### EXAMPLE 52

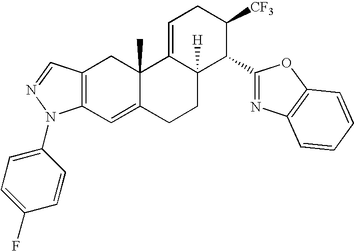

### (3R,4R,4aR,11aS)4-(1,3-benzoxazol-2-yl)-8-(4-fluorophenyl)-11a-methyl-3-(trifluoromethyl)-3,4,4a,5,6,8,11,11a-octahydro-2H-naphtho[1,2-f]indazole

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde (EXAMPLE 47) and 2-aminophenol according to the procedure described in EXAMPLE 51.

¹H NMR (CDCl₃, 500 MHz): δ 7.794 (m, 1H), 7.715 (m, 1H), 7.576 (m, 1H), 7.466-7.413 (m, 4H), 7.242 (t, J=8.2 Hz, 2H), 6.119 (s, 1H), 5.941 (d, J=5.5 Hz, 1H), 3.354 (m, 2H), 3.053 (m, 2H), 2.699 (d, J=16.1 Hz, 1H), 2.576 (m, 1H), 2.482 (m, 2H), 2.365 (m, 1H), 1.810 (m, 1H), 1.732 (m, 1H), 1.337 (s, 3H).

### EXAMPLE 53

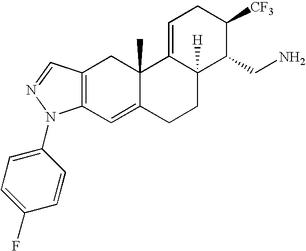

### 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanamine

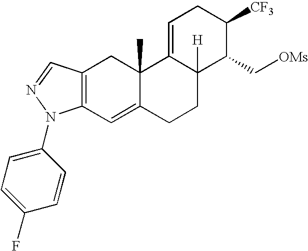

### Step A: [(11aS)-8-(4-fluorophenyl)-11a-methyl-3-(trifluoromethyl)-3,4,4a,5,6,8,11,11a-octahydro-2H-naphtho[1,2-f]indazol-4-yl]methyl methanesulfonate

To a solution of [(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanol from EXAMPLE 48 (17 mg, 0.039 mmol) in CH₂Cl₂ (2 mL) at 0° C. was added diisopropylethylamine (10.1 μL, 0.058 mmol) and methanesulfonyl chloride (36.4 μL, 0.047 mmol). After 2 hrs at 0° C. the reaction was diluted with ethyl acetate and washed with NaHCO₃, 1N HCl, brine, and was dried with MgSO₄. The solvent was removed under reduced pressure to yield the title compound. ¹H NMR (CDCl₃, 500 MHz): δ 7.515 (m, 2H), 7.444 (s, 1H), 7.197 (t, J=8.7 Hz, 2H), 6.248 (s, 1H), 5.786 (s, 1H), 4.359 (m, 2H), 3.035 (s, 3H), 2.864 (d, J=15.7 Hz, 1H), 2.580-2.309 (m, 7H), 2.141 (m, 1H), 2.049 (m, 1H), 1.565 (m, 1H), 1.257 (s, 3H).

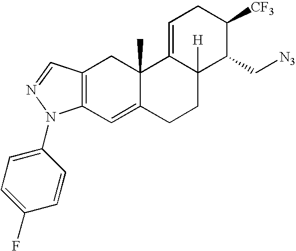

### Step B: (3R,4R4aR,11aS)-4-(azidomethyl)-8-(4-fluorophenyl)-11a-methyl-3-(trifluoromethyl)-3,4,4a,5,6,8,11a-octahydro-2H-naphtho[1,2-f]indazole

To a solution of [(11aS)-8-(4-fluorophenyl)-11a-methyl-3-(trifluoromethyl)-3,4,4a,5,6,8,11,11a-octahydro-2H-naphtho[1,2-f]indazol-4-yl]methyl methanesulfonate from Step A (120 mg, 0.234 mmol) in DMF (2.0 mL) was added sodium azide (20 mg, 0.375 mmol). The reaction mixture was heated to 50° C. for 4 hrs, cooled to RT, diluted with ethyl acetate. It was washed with H₂O, brine, and dried over MgSO₄. After removal of the solvent, the crude product was purified by column chromatography eluting with 5% to 100% ethyl acetate in hexane to yield 90 mg of the title compound. ¹H NMR (CDCl₃, 500 MHz): δ 7.482 (m, 2H), 7.431 (s, 1H), 7.179 (m, 2H), 6.237 (s, 1H), 5.748 (m, 1H), 3.654 (m, 1H), 3.554 (m, 1H), 2.838 (d, J=15.8 Hz, 1H), 2.222-2.504 (m, 6H), 2.066 (m, 1H), 1.868 (m, 1H), 1.521 (m, 1H), 1.238 (s, 3H).

### Step C: 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methanamine

To a solution of the intermediate from Step B (90 mg, 0.208 mmol) in THF/H₂O (2.08/0.23 mL) was added triphenylphosphine (107.6 mg, 0.171 mmol) at room temperature. The reaction mixture was heated to 50° C. for 4 hours, cooled to RT and the solvent was removed under reduced pressure. The crude material was purified by preparative thin layer chromatography eluting was 5% 2M NH₃ in MeOH/CH₂Cl₂ to obtain 40 mg of the title compound. ¹H NMR (CDCl₃, 500 MHz): δ 7.968 (bs, 2H), 7.556 (s, 1H), 7.450 (m, 3H), 7.217 (t, J=8.2 Hz, 2 H), 6.118 (s, 1H), 5.777 (s, 1H), 3.052 (m, 2H), 2.854 (m, 1H), 2.538 (m, 1H), 2.429 (m, 3H), 2.252 (m, 3H), 2.037 (m, 2H), 1.487 (m, 1H), 1.226 (s, 3H).

### EXAMPLE 54

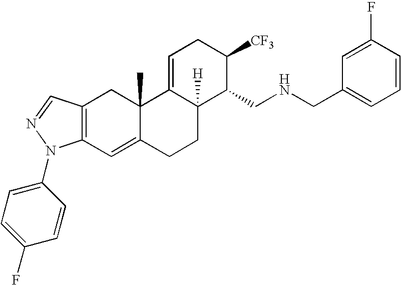

### 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]-N-(3-fluorobenzyl)methanamine

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 (11 mg, 0.0254 mmol) in 1,2-dichloroethane (0.5 mL) was added 3-fluorobenzylamine (4.80 mg, 0.381 mmol) at RT. After 2 hours, sodium triacetoxyborohydride (10.5 mg, 0.0381 mmol) was added and the reaction mixture was allowed to stir overnight. The reaction was quenched with NH₄Cl, diluted with CH₂Cl₂, and the organic layer was washed with H₂O, brine, and dried over MgSO₄. The crude product was purified via HPLC to yield the title compound. ¹H NMR (CDCl₃, 500 MHz): δ 7.473 (m, 2H), 7.398 (m, 1H), 7.232 (m, 1H), 7.169 (t, J=8.2 Hz, 2H), 7.074 (m, 2H), 6.917 (t, J=8.0 Hz, 1H), 6.202 (s, 1H), 5.676 (s, 1H), 3.815 (m, 1H), 3.743 (d, J=13.5 Hz, 1H), 2.760 (m, 2H), 2.503 (m, 3H), 2.379 (d, J=13.5 Hz, 1H), 2.232 (m, 4H), 1.860 (m, 1H), 1.529 (m, 2H), 1.201 (s, 3H).

### EXAMPLE 55

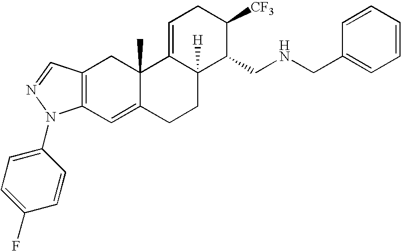

### 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]-N-benzylmethanamine

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and benzylamine according to the procedure described in EXAMPLE 54.

¹H NMR (CDCl₃, 500 MHz): δ 7.483 (m, 2H), 7.410 (s, 1H), 7.274 (m, 4H), 7.228 (m, 1H), 7.173 (t, J=8.5 Hz, 2H), 6.197 (s, 1H), 5.666 (s, 1H), 3.806 (m, 2H), 2.775 (m, 2H), 2.584 (m, 2H), 2.485 (m, 1H), 2.338 (d, J=15.6 Hz, 1H), 2.247 (m, 4H), 1.870 (m, 1H), 15.42 (m, 1H), 1.201 (s, 3H).

### EXAMPLE 56

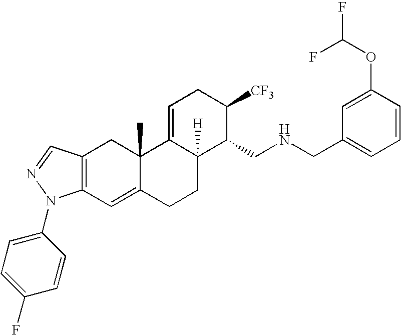

### 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-1yl]-N-benzylmethanamine

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and 3-(difluoromethoxy)benzylamine according to the procedure described in EXAMPLE 54. ¹H NMR (CDC₃, 500 MHz): δ 7.472 (m, 2H), 7.405 (s, 1H), 7.269 (m, 1H), 7.170 (m, 3H), 7.096 (s, 1H), 6.988 (d, J=8.0 Hz, 1H), 6.471 (t, J_{HF}=74.2 Hz, 1H), 6.203 (s, 1H), 5.677 (s, 1H), 3.815 (m, 2H), 2.778 (m, 2H), 2.570 (m, 3H), 2.347 (d, J=15.8 Hz, 1H), 2.264 (m, 4H), 1.852 (m, 1H), 1.556 (m, 1H).

### EXAMPLE 57

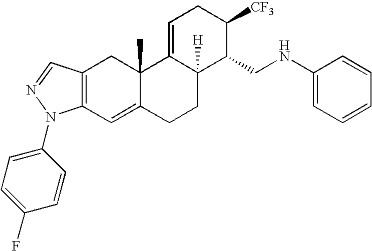

### 1-[(3R,4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]-N-benzylmethanamine

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and aniline according to the procedure described in EXAMPLE 54. ¹H NMR (CDCl₃, 500 MHz): δ 7.588 (s, 1H), 7.456 (m, 2H), 7.215 (m, 4H), 6.914 (t, J=8.1 Hz, 1H), 6.806 (d, J=6.806. 2H), 6.137 (s, 1H), 5.772 (s, 1H), 5.051 (bs, 1H), 3.305 (m, 2H), 2.829 (d, J=15.8 Hz, 1H), 2.497 (m, 1H), 2.436 (d, J=15.8 Hz, 1H), 2.365 (m, 1H), 2.278 (m, 1H), 2.156 (m, 1H), 2.006 (m, 1H), 1.662 (m, 1H), 1.240 (s, 3H).

### EXAMPLE 58

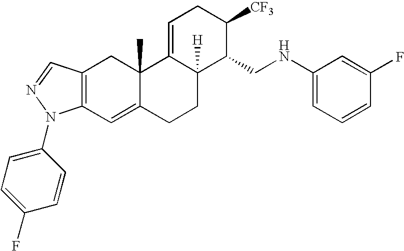

### N-{[(3R4R,4aR,11aS)-8-(4-fluorophenyl)-11a-methyl-3-(trifluoromethyl)-3,4,4a,5,6,8,11,11a-octahydro-2H-naphtho[1,2-f]indazol-4-yl]methyl}-3-fluoroaniline

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and 3-fluoroaniline according to the procedure described in EXAMPLE 54. ¹H NMR (CDCl₃, 500 MHz): δ 7.620 (s, 1H), 7.438 (m, 2H) 7.198 (t, J=6.4 Hz, 2H), 7.087 (m, 1H), 6.388 (m, 2H), 6.311 (m, 1H, 6.121 (s, 1H), 5.771 (s, 1H), 4.817 (bs, 1H), 3.214 (d, J=5.5 Hz, 2H), 2.778 (d, J=15.8 Hz, 1H), 2.453 (d, 15.8 Hz, 1H), 2.408 (m, 2H), 2.352 (m, 2H), 2.297 (m, 2H), 2.095 (m, 1H), 1.985 (m, 1H), 1.683 (m, 1H), 1.238 (s, 3H).

### EXAMPLE 59

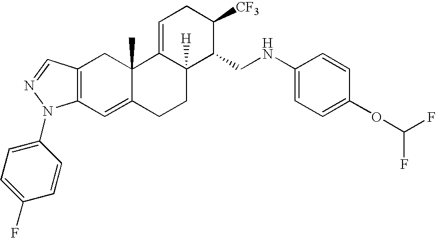

### N-{[(3R,4R,4aR,11aS)-8-(4-fluorophenyl)-11a-methyl-4-(trifluoromethyl)-3,4,4a,5,6 8,11,11a-octahydro-2H-naphtho[1,2-f]indazol-4-yl]methyl}-3-(difluoromethoxy)aniline

The title compound was prepared from (3R,4R,4aR,11as)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and 4-difluoromethoxy aniline according to the procedure described EXAMPLE 54. ¹H NMR (CDCl₃, 500 MHz): δ 7.861 (bs, 1H), 7.681 (s, 1H), 7.458 (m, 2H), 7.250 (t, J=14.0 Hz, 2H), 7.160 (t, J=8.0 Hz, 1H), 6.634-6.336 (m, 3H), 6.129 (s, 1H), 5.791 (s, 1H), 3.265 (d, J=4.6 Hz, 2H), 2.823 (d, J=16.4 Hz, 1H), 2.514-2.318 (m, 7H), 2.117 (m, 1H), 2.022 (m, 1H), 1.700 (m, 1H), 1.266 (s, 3H).

### EXAMPLE 60

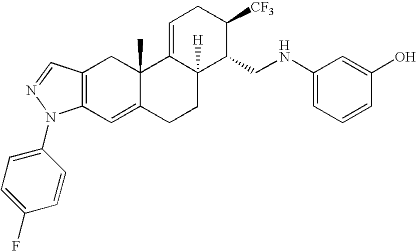

### 3-({[(3R,4R,4aR,11aS)-8-(4-fluorophenyl)-11a-methyl-3-(trifluoromethyl)-3,4a,5,6,8,11,11a-octahydro-2H-naphtho[1,2-f]indazol-4-yl]methyl}amino)phenol

The title compound was prepared from (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbaldehyde from EXAMPLE 47 and 3-hydroxyaniline according to the procedure described in EXAMPLE 54. ¹H NMR (CD₃OD, 500 MHz): δ 10.061 (s, 1H), 7.477 (m, 1H), 7.449 (s, 1H), 7.249 (t, J=12.8 Hz, 2H), 7.117 (m, 2H), 6.915 (d, J=8.0 Hz, 1H), 6.558 (dd, J=1.8 Hz, 8.0 Hz, 1), 6.221 (s, 1H), 5.860 (m, 1H), 2.898 (d, J=15.8 Hz, 1H), 2.782 (m, 1H), 2.689 (m, 1H), 2.545 (m, 2H), 2.432 (m, 3H), 2.244 (m, 2H), 1.978 (m, 1H), 1.602 (m, 1H), 1.251 (s, 1H).

### EXAMPLE 61

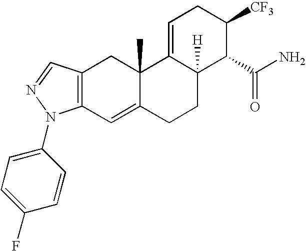

### (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxamide

To a stirred solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid from EXAMPLE 2 Step A (45 mg, 0.10 mmol) in DMF (600 μl) was added benzotriazol-1-yloxy-tris(dimethylamino)phosphonium hexafluorophosphate (66 mg, 0.15 mmol), 1-hydroxybenzotriazole hydrate (20 mg, 0.15 mmol), N,N-diisopropylethylamine (70 μl, 0.40 mmol), and NH₄Cl (11 mg, 0.20 mmol). The reaction mixture was stirred under N₂ at room temperature for 2 hours. It was then diluted with ethyl acetate and washed 2× with water followed by brine. The organic layer was dried over Na₂SO₄ and concentrated under reduced pressure. The product was purified by preparative thin-layer chromatography, eluting with 3:2 ethyl acetate-hexanes to yield 32 mg of the title compound.

Mass spectrum (ESI) 446 (M+1).

¹H NMR (500 MHz, CD₃OD): δ 1.26 (s, 3H); 1.59 (m, 1H); 2.01 (m, 1H); 2.24 (m, 1H); 2.42 (m, 4H); 2.60 (½ ABq, J=15.8 Hz, 1H); 2.67 (m, 2H); 2.88 (½ ABq, J=15.8 Hz, 1H); 5.85 (br d, J=6.2 Hz, 1H); 6.25 (s, 1H); 7.28 (m, 2H); 7.44 (s, 1H); 7.49 (m, 2H).

### EXAMPLE 62

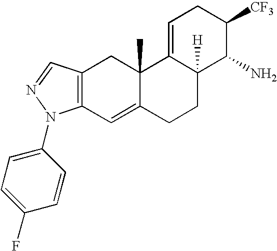

### (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine

To a stirred solution of 3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxamide from EXAMPLE 61 (268 mg, 0.602 mmol) in 1,4-dioxane (4.5 ml), under N₂, was added NaOCl (10-13%) (1.2 ml), 2N aqueous NaOH (2.1 ml), and water (2.1 ml). The reaction mixture was heated at 40° C. for 1 hour. The reaction mixture was diluted with water and extracted 3× with ethyl acetate. The combined organic extracts were washed with brine, dried over Na₂SO₄, and concentrated under reduced pressure to yield 226 mg of the title compound.

Mass spectrum (ESI) 418 (M+1).

¹H NMR (500 MHz, CD₃OD): δ 1.25 (s, 3H); 1.61 (m, 1H); 2.32 (m, 7H); 2.52 (½ ABq, J=15.8 Hz, 1H); 2.88 (½ ABq, J=15.8 Hz, 1H); 2.95 (app t, J=8.0 Hz, 1H); 5.81 (br d, J=6.0 Hz, 1H); 6.25 (s, 1H); 7.29 (m, 2H); 7.43 (s, 1H); 7.49 (m, 2H).

### EXAMPLE 63

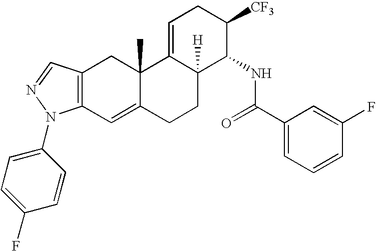

### N-[(3R,4R,4aS,11 aS)-8-(4-fluorophenyl)-11a-methyl-3-(trifluoromethyl)-3,4,4a,5,6,8,11,11a-octahydro-2H-naphtho[1,2-f]indazol-4-yl]-3-fluorobenzamide

To a stirred solution of (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 (10 mg, 0.024 mmol) in methylene chloride (0.5 mL) was added diisopropylethylamine (8.4 μl, 0.048 mmol) followed by 3-fluorobenzoyl chloride (4.4 μl, 0.036 mmol). The reaction mixture was stirred under N₂ at room temperature for 35 min. It was then concentrated under reduced pressure, diluted with ethyl acetate, and washed with saturated NaHCO₃, 2N aqueous HCl, and brine. The organic layer was dried over Na₂SO₄, and concentrated under reduced pressure. The product was purified by preparative thin-layer chromatography, eluting with 95:5 dichloromethane-methanol to yield 8 mg of the title compound.

Mass spectrum (ESI) 540 (M+1).

¹H NMR (500 MHz, CD₃OD): δ 1.28 (s, 3H); 1.72 (m, 1H); 2.03 (m, 1H); 2.44 (m, 4H); 2.61 (½ ABq, J=15.8 Hz, 1H); 2.70 (m, 2H); 2.89 (½ ABq, J=15.8 Hz, 1H); 4.27 (app t, J=10.9 Hz, 1H); 5.87 (br d, J=6.0 Hz, 1H); 6.24 (s, 1H); 7.29 (m, 3H); 7.44 (s, 1H); 7.51 (m, 4H); 7.64 (br d, J=8.0 Hz, 1H).

### EXAMPLE 64

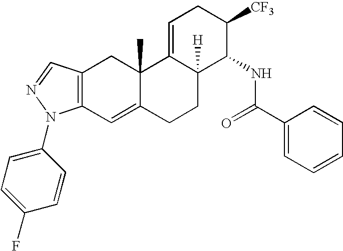

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]benzamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and benzoyl chloride according to the procedure described in EXAMPLE 63.

Mass spectrum (ESI) 522 (M+1).

¹H NMR (500 MHz, CD₃OD): δ 1.28 (s, 3H); 1.73 (m, 1H); 2.04 (m, 1H); 2.43 (m, 4H); 2.61 (½ ABq, J=15.8 Hz, 1H); 2.70 (m, 2H); 2.90 (½ ABq, J=15.8 Hz, 1H); 4.28 (app t, J=10.8 Hz, 1H); 5.87 (br d, J=6.0 Hz, 1H); 6.24 (s, 1H); 7.28 (app t, J=8.7 Hz, 2H); 7.44 (s, 1H); 7.48 (m, 4H); 7.55 (app t, J=7.1 Hz, 1H); 7.81 (br d, J=7.5 Hz, 2H).

### EXAMPLE 65

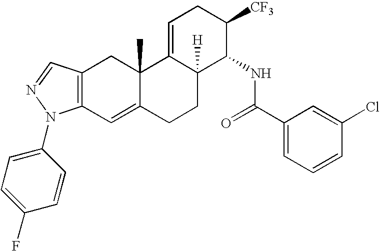

### N-[(3R,4R,4aS,11aS)-8-(4-fluorophenyl)-11a-methyl-3-(trifluoromethyl)-3,4,4a,5,5,6,8,8,11,11a-octahydro-2H-naphtho[1,2-f]indazol-4-yl]-3-chlorobenzamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-chlorobenzoyl chloride according to the procedure described in EXAMPLE 63. Triethylamine was used as the base.

Mass spectrum (ESI) 556 (M+1).

¹H NMR (500 MHz, CD₃OD): δ 1.24 (s, 3H); 1.68 (m, 1H); 1.99 (m, 1H); 2.36 (m, 3H); 2.48 (m, 1H); 2.57 (½ ABq, J=15.8 Hz, 1H); 2.65 (m, 2H); 2.85 (½ ABq, J=15.8 Hz, 1H); 4.23 (app t, J=10.3 Hz, 1H); 5.83 (br d, J=6.2 Hz, 1H); 6.20 (s, 1H); 7.24 (m, 2H); 7.41 (s, 1H); 7.44 (m, 3H); 7.53 (m, 1H); 7.70 (br d, J=8.0 Hz, 1H); 7.78 (app t, J=1.7 Hz, 1H).

### EXAMPLE 66

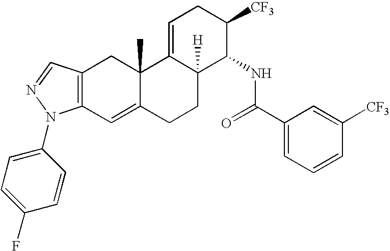

### N-[(3R,4R,4aS,11aS-8-(4-fluorophenyl)-11a-methyl-3-(trifluoromethyl)-3,4,4a,5,6,8,11,11a-octahydro-2H-naphtho[1,2-f]indazol-4-yl]-3-(trifluoromethyl)benzamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-trifluoromethylbenzoyl chloride according to the procedure described in EXAMPLE 63. Triethylamine was used as the base.

Mass spectrum (ESI) 590 (M+1).

¹H NMR (500 MHz, CD₃OD): δ 1.25 (s, 3H); 1.69 (m, 1H); 2.01 (m, 1H); 2.36 (m, 3H); 2.49 (m, 1H); 2.58 (½ ABq, J=15.8 Hz, 1H); 2.67 (m, 2H); 2.86 (½ ABq, J=15.8 Hz, 1H); 4.26 (app t, J=10.8 Hz, 1H); 5.84 (br d, J=6.1 Hz, 1H); 6.21 (s, 1H); 7.24 (app t, J=8.7 Hz, 2H); 7.41 (s, 1H); 7.45 (m, 2H); 7.67 (app t, J=7.7 Hz, 1H); 7.83 (br d, J=7.7 Hz, 1H); 8.04 (br d, J=7.8 Hz, 1H); 8.09 (s,1H).

### EXAMPLE 67

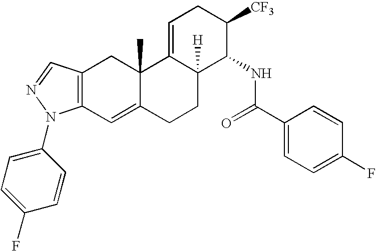

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]4-fluorobenzamide

The title compound was prepared from (3R,4R,4aS,11aS)-8-(4-fluorophenyl)-11a-methyl-3 -(trifluoromethyl)-3,4,4a,5,6,8,11,11a-octahydro-2H-naphtho[1,2-f]indazol-4-amine from EXAMPLE 62 and 4-fluorobenzoyl chloride according to the procedure described in EXAMPLE 63.

Mass spectrum (ESI) 540 (M+1).

### EXAMPLE 68

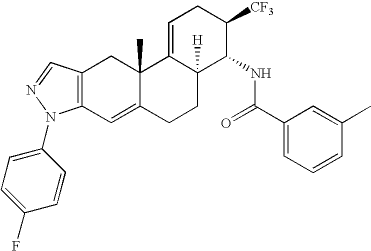

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-3-methylbenzamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-methylbenzoyl chloride according to the procedure described in EXAMPLE 63.

Mass spectrum (ESI) 536 (M+1).

### EXAMPLE 69

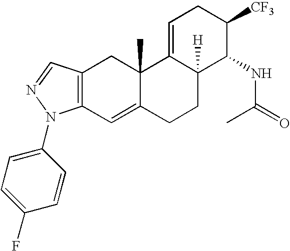

### N-[(3R,4R,4aS,11aS)-8-(4-fluorophenyl)-11a-methyl-3-(trifluoromethyl-3,4,4a,5,6,8,11,11a-octahydro-2H-naphtho[1,2-f]indazol-4-yl]acetamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and acetyl chloride according to the procedure described in EXAMPLE 63.

Mass spectrum (ESI) 460 (M+1).

### EXAMPLE 70

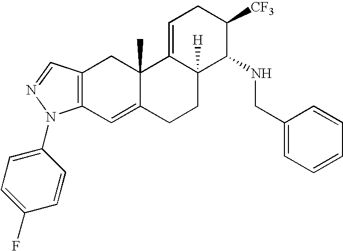

### (3R,4R,4aS,11aS)-N-benzyl-8-(4-fluorophenyl)-11a-methyl-3-(trifluoromethyl)-3,4,4a,5,6,8,11,11a-octahydro-2H-naphtho[1,2-f]indazol-4-amine

To a mixture of (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 (15 mg, 0.036 mmol) in 1,2-dichloroethane (0.5 ml) was added benzaldehyde (5 μl, 0.047 mmol) followed by glacial acetic acid (2 μl, 0.03.6 mmol). After stirring under N₂ for 30 minutes at room temperature, sodium triacetoxyborohydride (15 mg, 0.072 mmol) was added, and the mixture was stirred at room temperature overnight. The reaction mixture was quenched with 4 ml of 2N aqueous NaOH and extracted 3× with dichloromethane. The combined organic extracts were washed with brine, dried over Na₂SO₄, and concentrated under reduced pressure. The product was purified by flash column chromatography on silica gel eluting with 10%-100% ethyl acetate in hexanes, followed by a second purification by preparative thin-layer chromatography, eluting with 2:3 ethyl acetate-hexanes to yield 9 mg of the title compound.

Mass spectrum (ESI) 508 (M+1).

¹H NMR (500 MHz, CD₃OD): δ 1.22 (s, 3H); 1.55 (m, 1H); 2.02 (m, 1H); 2.27 (m, 3H); 2.47 (m, 2H); 2.49 (½ ABq, J=15.8 Hz, 1H); 2.59 (m, 1H); 2.86 (½ ABq, J=15.8 Hz, 1H); 2.93 (app t, J=6.8 Hz, 1H); 3.82(Abq, J=13.0 Hz, J=26.1 Hz, 2H); 5.77 (br s, 1H); 6.23 (s, 1H); 7.22 (m, 1H); 7.29 (m, 4H); 7.34 (br d, J=7.5 Hz, 2H); 7.43 (s, 1H); 7.49 (m, 2H).

### EXAMPLE 71

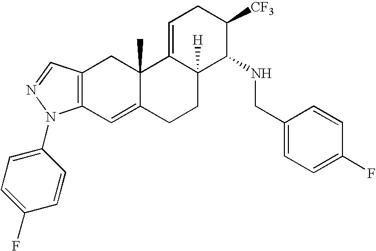

### (3R,4R,4aS, 11aS)-N-(4-fluorobenzyl)-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 4-fluorobenzaldehyde according to the procedure described in EXAMPLE 70.

Mass spectrum (ESI) 526 (M+1).

¹H NMR (500 MHz, CD₃OD): δ 1.22 (s, 3H); 1.57 (m, 1H); 2.04 (m, 1H); 2.27 (m, 3H); 2.47 (m, 2H); 2.49 (½ ABq, J=15.8 Hz, 1H); 2.58 (m, 1H); 2.85 (½ ABq, J=15.8 Hz, 1H); 2.91 (app t, J=7.3 Hz, 1H); 3.81(Abq, J=13.0 Hz, J=25.8 Hz, 2H); 5.77 (br s, 1H); 6.23 (s, 1H); 7.02 (m, 2H); 7.28 (m, 2H); 7.36 (m, 2H); 7.43 (s, 1H); 7.49 (m, 2H).

### EXAMPLE 72

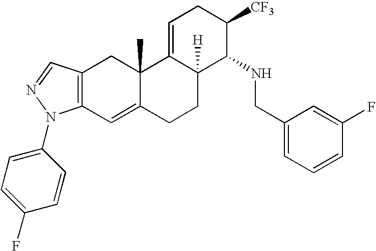

### (3R,4R,4aS,11aS)-N-(3-fluorobenzyl)-8-(4-fluorophenyl)-11a-methyl-3-(trifluoromethyl)-3,4,4a,5 6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-fluorobenzaldehyde according to the procedure described in EXAMPLE 70.

Mass spectrum (ESI) 526 (M+1).

¹H NMR (500 MHz, CD₃OD): δ 1.22 (s, 3H); 1.58 (m, 1H); 2.05(m, 1H); 2.27 (m, 3H); 2.47 (m, 2H); 2.49 (½ ABq, J=15.8 Hz, 1H); 2.58 (m, 1H); 2.86 (½ ABq, J=15.8 Hz, 1H); 2.92 (app t, J=5.9 Hz, 1H); 3.84(Abq, J=14.0 Hz, J=25.0 Hz, 2H); 5.77 (br s, 1H); 6.23 (s, 1H); 6.94 (m, 1H); 7.14 (m, 2H); 7.29 (m, 3H); 7.42 (s, 1H); 7.48 (m, 2H).

### EXAMLE 73

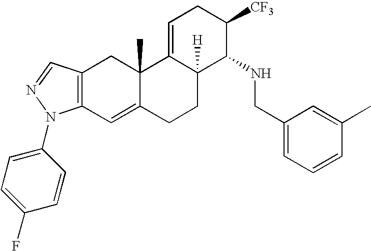

### (3R,4R,4aS,11aS)-8-(4-fluorophenyl)-11a-methyl-N-(3-methylbenzyl)-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine EXAMPLE 62 and 3-methylbenzaldehyde according to the procedure described in EXAMPLE 70.

Mass spectrum (ESI) 521(M+1).

¹H NMR (500 MHz, CD₃OD): δ 1.22 (s, 3H); 1.55 (m, 1H); 1.99 (m, 1H); 2.27 (m, 3H); 2.30 (s, 3H); 2.47 (½ ABq, J=15.8 Hz, 1H); 2.48 (m, 2H); 2.59 (m, 1H); 2.84 (½ ABq, J=15.8 Hz, 1H); 2.94 (app t, J=5.5 Hz, 1H); 3.78 (Abq, J=12.8 Hz, J=32.0 Hz, 2H); 5.77 (br s, 1H); 6.22 (s, 1H); 7.04 (br d, J=7.6 Hz, 1H); 7.12 (br d, J=7.7 Hz, 1H); 7.18 (m, 2H); 7.28 (app t, J=8.5 Hz, 2H); 7.43 (s, 1H); 7.49 (m, 2H).

### EXAMPLE 74

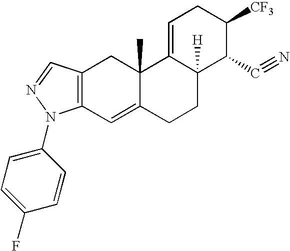

### (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carbonitrile

To a solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxamide from EXAMPLE 61 (20 mg) in DMF (200 μl) was added cyanuric chloride (8 mg). The reaction mixture was stirred under N₂ at room temperature for 1.75 hours. It was then diluted with water and extracted 3× with ethyl acetate. The combined organic layers were washed with brine, dried over Na₂SO₄, and concentrated under reduced pressure. The product was purified by flash column chromatography on silica gel eluting with 10%-100% ethyl acetate in hexanes to yield 13 mg of the title compound.

Mass spectrum ESI) 428 (M+1).

¹H NMR (500 MHz, CD₃OD): δ 1.26 (s, 3H); 1.72 (m, 1H); 2.29 (m, 311); 2.45 (m, 2H); 2.59 (½ ABq, J=15.8 Hz, 1H); 2.87 (m, 4H); 5.87 (br d, J=6.0 Hz, 1H); 6.30 (s, 1H); 7.29 (m, 2H); 7.44 (s, 1H); 7.49 (m, 2H).

### EXAMPLE 75

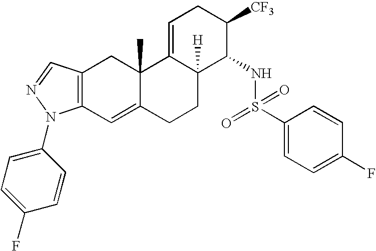

### N-[(3R4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl-]-4-fluorobenzenesulfonamide

To a mixture of (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 (15 mg, 0.036 mmol) in THF (0.5 ml) was added 4-fluorobenzenesulfonyl chloride (11 mg, 0.054 mmol) followed by diisopropylethylamine (9 μl, 0.054 mmol). After stirring under N₂ at room temperature for ca. 2 hours, the solvent was removed under reduced pressure. The product was purified by preparative thin-layer chromatography, eluting with 95:5 dichloromethane-methanol to yield 12 mg of the title compound.

Mass spectrum (ESI) 576 (M+1).

¹H NMR (500 MHz, CD₃OD): δ 1.20 (s, 3H); 1.52 (m, 1H); 1.76 (m, 1H); 2.24 (m, 4H); 2.48 (½ ABq, J=15.8 Hz, 1H); 2.50 (m, 2H); 2.71 (½ ABq, J=15.8 Hz, 1H); 3.60 (app t, J=8.0 Hz, 1H); 5.78 (br s, 1H); 6.17 (s, 1H); 7.23 (app t, J=8.7 Hz, 2H); 7.29 (app t, J=8.7 Hz, 2H); 7.43 (s, 1H); 7.48 (m, 2H); 7.89 (m, 2H).

### EXAMPLE 76

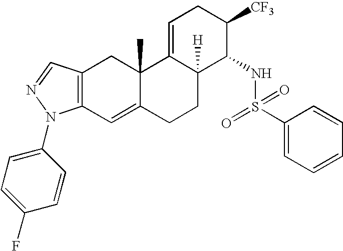

### N-[(3R4R,4aS,11aS)-8-(4-fluorophenyl)-11a-methyl-3-(trifluoromethyl-3,4,4a,5,6,8,11,11a-octahydro-2H-naphtho[1,2-f]indazol-4-yl]benzenesulfonamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and benzenesulfonyl chloride according to the procedure described in EXAMPLE 75.

Mass spectrum (ESI) 558 (M+1).

### EXAMPLE 77

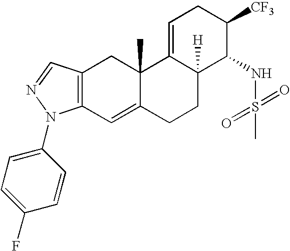

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]methanesulfonamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and methanesulfonyl chloride according to the procedure described in EXAMPLE 75.

Mass spectrum (ESI) 496 (M+1).

### EXAMPLE 78

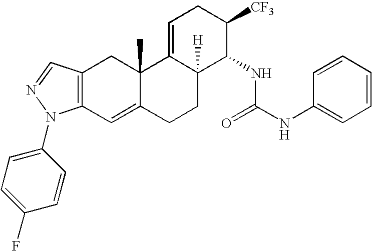

### N-[(3R,4R,4aS,11aS)-8-(4-fluorophenyl)-11a-methyl-3-(trifluoromethyl)-3,4,4a,5,6,8 11,11a-octahydro-2H-naphtho[1,2-f]indazol-4-yl]-N′-phenylurea

To a solution of (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 (15 mg, 0.036 mmol) in dichloromethane (0.5 ml) was added phenyl isocyanate (4.1 μl, 0.038 mmol). After stirring under N₂ at room temperature for 3 hours, the solvent was removed under reduced pressure. The product was purified by flash column chromatography on silica gel eluting with 1%-100% methanol in dichloromethane, followed by a second purification by preparative thin-layer chromatography, eluting with 95:5 dichloromethane-methanol to yield 13 mg of the title compound.

Mass spectrum (ESI) 537 (M+1).

¹H NMR (500 MHz, CD₃OD): δ 1.27 (s, 3H); 1.72 (m, 1H); 2.10 (m, 1H); 2.41 (m, 4H); 2.59 (m, 2H); 2.59 (½ ABq, J=15.8 Hz, 1H); 2.89 (½ ABq, J=15.8 Hz, 1H); 3.93 (app t, J=9.6 Hz, 1H); 5.83 (br d, J=5.3 Hz, 1); 6.25 (s, 1H); 6.99 (app t, J=6.9 Hz, 1H); 7.27 (m, 4H); 7.34 (br d, J=8.3 Hz, 2H); 7.44 (s, 1H); 7.49 (m, 2H).

### EXAMPLE 79

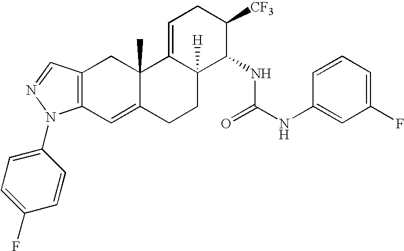

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-N′-(3-fluorophenyl)urea

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-fluorophenylisocyanate according to the procedure described in EXAMPLE 78.

Mass spectrum (ESI) 555 (M+1).

¹H NMR (500 MHz, CD₃OD): δ 1.27 (s, 3H); 1.71 (m, 1H); 2.09 (m, 1H); 2.41 (m, 4H); 2.57 (m, 2H); 2.59 (½ ABq, J=15.8 Hz, 1H); 2.88 (2 ABq, J=15.8 Hz, 3.93 (app t, J=9.6 Hz, 1H); 5.83 (br d, J=6.0 Hz, 1H); 6.24 (s, 1H); 6.69 (m, 1H); 7.02 (br d, J=8.3 Hz, 1H); 7.23 (dd, J=8.2 Hz, J=14.8 Hz, 1H); 7.28 (app t, J=8.4 Hz, 2H); 7.33 (m, 1H); 7.44 (s, 1H); 7.49 (m, 2H).

### EXAMPLE 80

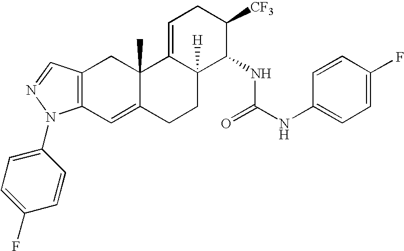

### N-[(3R,4R,4aS,11aS)-8-(4-fluorophenyl)-11a-methyl-3-(trifluoromethyl)-3,4,4,4a,5,6,8,11,11a-octahydro-2H-naphtho[1,2-f]indazol-4-yl]-N′-(4-fluorophenyl)urea

The tide compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 4-fluorophenylisocyanate according to the procedure described in EXAMPLE 78.

Mass spectrum (ESI) 555 (M+1);

¹H NMR (500 MHz, CD₃OD): δ 1.26 (s, 3H); 1.71 (m, 1H); 2.09 (m, 1H); 2.41 (m, 4H); 2.57 (m, 2H); 2.59 (½ ABq, J=15.8 Hz, 1H); 2.88 (½ ABq, J=15.8 Hz, 1H); 3.92 (app t, J=9.9 Hz, 1H); 5.82 (br d, J=5.7 Hz, 1H); 6.24 (s, 1H); 7.00 (app t, J=8.9 Hz, 2H); 7.28 (app t, J=9.0 Hz, 2H); 7.33 (m, 2H); 7.44 (s, 1H); 7.49 (m, 2H).

### EXAMPLE 81

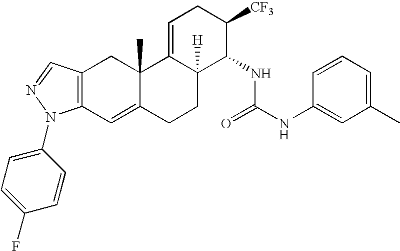

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-N′-(3-methylphenyl)urea

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine from EXAMPLE 62 and 3-methylphenylisocyanate according to the procedure described in EXAMPLE 78.

Mass spectrum (ESI) 551 (M+1).

¹H NMR (500 MHz, CD₃OD): δ 1.26 (s, 3H); 1.71 (m, 1H); 2.09 (m, 1H); 2.30 (s, 3H); 2.42 (m, 4H); 2.57 (m, 2H); 2.58 (½ ABq, J=15.8 Hz, 1H); 2.88 (½ ABq, J=15.8 Hz, 1H); 3.92 (app t, J=10.0 Hz, 1H); 5.82 (br d, J=5.7 Hz, 1H); 6.24 (s, 1H); 6.82 (app t, J=3.2 Hz, 1H); 7.13 (br d, J=4.8 Hz, 2H); 7.19 (s, 1H); 7.28 (app t, J=8.7 Hz, 2H); 7.44 (s, 1H); 7.49 (m, 2H).

### EXAMPLES 82 AND 83

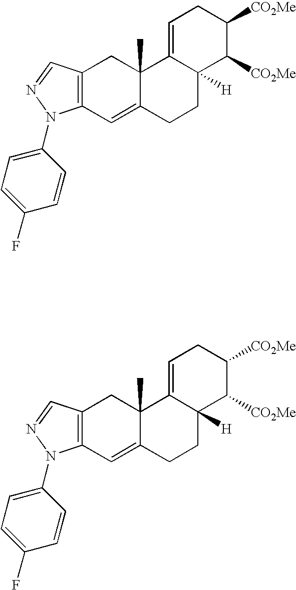

### Dimethyl (3R,4S,4aR,11aS)-8-(4-fluorophenyl)-11a-methyl-3,4,4a,5,6,8,11,11a-octahydro-2H-naphtho[1,2-f]indazole-3,4-dicarboxylate and dimethyl (3S,4R,4aS,11aS)-8-(4-fluorophenyl)-11a-methyl-3,4,4a,5,6,8,11,11a-octahydro-2H-naphtho[1,2-f]indazole-3,4-dicarboxylate

To a solution of 100 mg of (4aS)-1-(4-fluorophenyl)-4a-methyl-5-vinyl4,4a,7,8-tetrahydro-1H-benzo[f]indazole prepared as in EXAMPLE 1 Step F in 2 mL of CH₂C₂ was added 39 mg of maleic anhydride, then 0.7 mL of a 1.0 M solution of BCl₃ in CH₂CH₂. The mixture was stirred at room temperature for 2 h, then concentrated. The residue was dissolved in 3 mL of 2:1 benzene-MeOH and excess TMSCH₂N₂ was added. The mixture was stirred overnight, then quenched with trifluoroacetic acid and concentrated. Preparative TLC, eluting with 1:1 hexanes-EtOAc, provided 92 mg of the title compounds as approximately a 2:1 mixture. Further purification by HPLC on Chiralcel OJ, eluting with 20% ethanol-heptane, provided two clean diastereomers: 29 mg of dimethyl (3R,4S,4aR,11aS)-8-(4-fluorophenyl)-11a-methyl-3,4,4a,5,6,8,11,11a-octahydro-2H-naphtho[1,2-f]indazole-3,4-dicarboxylate and 14.5 mg of dimethyl (3S,4R,4aS,11aS)-8-(4-fluorophenyl)-11a-methyl-3,4,4a,5,6,8,11,11a-octahydro-2H-naphtho[1,2-f]indazole-3,4-dicarboxylate. Mass spectrum (ESI) 451 (M+1). ¹H NMR (500 MHz, CD₃CN) diagnostic peaks only:

EXAMPLE 82 Dimethyl (3R,4S,4aR,11aS)-8-(4-fluorophenyl)-11a-methyl-3,4,4a,5,6,8,11,11a-octahydro-2H-naphtho[1,2-f]indazole-3,4-dicarboxylate δ 3.13 (dd, J=4 Hz, 6 Hz, 1H, H₄), 2.98 (ddd, J=4 Hz, 7.5 Hz, 7.5 Hz, 1H, H₃) 2.69-2.79 (m, 2H, H₄ₐ, H₁₁).

EXAMPLE 83 Dimethyl (3S,4R,4aS,11aS)-8-(4-fluorophenyl)-11a-methyl-3,4,4a,5,6,8,11,11a-octahydro-2H-naphtho[1,2-f]indazole-3,4-dicarboxylate: δ 3.22 (dd, J=4 Hz, 6 Hz, 1H, H₄), 3.01 (d, J=15 Hz, 1H, H₁₁), 2.78-2.87 (m, 2H, H₃, H₄ₐ).

### EXAMPLES 84, 85 AND 86

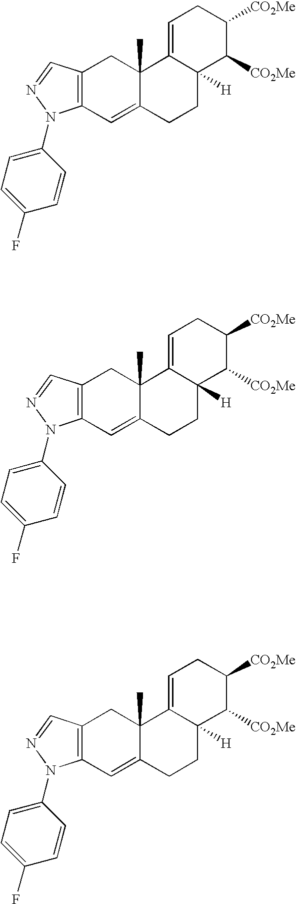

### Dimethyl (3S,4S,4aR,11aS)-8-(4-fluorophenyl)-11a-methyl-3,4,4a,5,6,8,11,11a-octahydro-2H-naphtho[1,2-f]indazole-3,4-dicarboxylate; dimethyl (3S,4S,4aS,11aS)-8-(4-fluorophenyl)-11a-methyl-3,4,4a,5,5,6,8,11,11a-octahydro-2H-naphtho[1,2-f]indazole-3,4-dicarboxylate: and dimethyl (3R,4R,4aR,11aS)-8-(4-fluorophenyl)-11a-methyl-3,4,4a,5,6,8,11,11a-octahydro-2H-naphtho[1,2-f]indazole-3,4-dicarboxylate

The title compounds were prepared from (4aS)-1-(4-fluorophenyl)-4a-methyl-5-vinyl-4,4a,7,8-tetrahydro-1H-benzo[f]indazole EXAMPLE 1 Step F and dimethyl fumarate according to the procedure described in EXAMPLE 82.

Purification by HPLC on Chiralpak AD, eluting with 15% ethanol-heptane provided three clean diastereomers. Mass spectrum (ESI) 451 (M+1). ¹H NMR (500 MHz, C₆D₆) diagnostic peaks only:

EXAMPLE 84 Dimethyl (3S,4S,4aR,11aS)-8-(4-fluorophenyl)-11a-methyl-3,4,4a,5,6,8,11,11a-octahydro-2H-naphtho[1,2-f]indazole-3,4-dicarboxylate: δ 3.19 S (dd, J=6 Hz, 12 Hz, 1H, H₄), 2.95 (ddd, J=5.5 Hz, 11.5 Hz, 12 Hz, 1H, H₃) 2.84 (m, 1H, H₄ₐ).

EXAMPLE 85 Dimethyl (3S,4S,4aS,11aS)-8-(4-fluorophenyl)-11a-methyl-3,4,4a,5,6,8,11,11a-octahydro-2H-naphtho[1,2-f]indazole-3,4-dicarboxylate: δ 3.00 (ddd, J=5 Hz, 10.5 Hz, 11 Hz, 1H, H₃), 2.73 (d, J=15 Hz, 1H, H₁₁), 2.66 (m, 2H, H₁₄, H₄ₐ).

EXAMPLE 86 Dimethyl (3R,4R,4aR,11aS)-8-(4-fluorophenyl)-11a-methyl-3,4,4a,5,6,8,11,11a-octahydro-2H-naphtho[1,2-f]indazole-3,4-dicarboxylate: δ 2.95 (ddd, J=6 Hz, 9.5 Hz, 10.5 Hz, 1H, H₃), 2.78 (dd, J=9.5 Hz, 11 Hz, 1H, H₄), 2.67 (m, 1H, H₁₁).

### EXAMPLES 87, 88, 89, AND 90

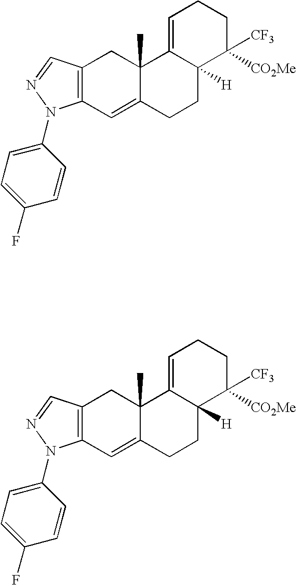

### Methyl (4aS,11aS)-8-(4-fluorophenyl)-11a-methyl-4-(trifluoromethyl)-3,4,4a,5,6,8,11,11a-octahydro-2H-naphtho[1,2-f]indazole-4-carboxylate and methyl (4aR,11aS)-8-(4-fluorophenyl)-11a-methyl-4-(trifluoromethyl)-3,4,4a,5,6,8,11,11a-octahydro-2H-naphtho[1,2-f]indazole-4-carboxylate

The title compounds were prepared from (4aS)-1-(4-fluorophenyl)-4a-methyl-5-vinyl-4,4a,7,8-tetrahydro-1H-benzo[f]indazole EXAMPLE 1 Step F and trifluoromethyl acrylic acid according to the procedure described in EXAMPLE 82. Purification by HPLC on Chiralcel OJ, eluting with 10% ethanol-heptane, provided four clean diastereomers, two with 4aS stereochemistry and two with 4aR stereochemistry. Mass spectrum (ESI) 447 (M+1). ¹H NMR (600 MHz, C₆D₆) diagnostic peaks only:

EXAMPLE 87 Methyl (4aS,11aS)-8-(4-fluorophenyl)-11a-methyl-4-(trifluoromethyl)-3,4,4a,5,6,8,11,11a-octahydro-2H-naphtho[1,2-f]indazole-4-carboxylate, diastereomer 1: δ 3.09 (br t, J=9.6 Hz, 1H, H₄ₐ), 2.91 (d, J=15.6 Hz, 1H, H₁₁), 2.25 (dd, J=5 Hz, 12 Hz, 1H, H₃) 2.17, (d, J=15.6 Hz, 1H, H₁₁), 2.09 (m, 1H, H₂), 2.01 (m, 1H, H₃), 1.83-1.94 (m, 2H, H₂, H₆).

EXAMPLE 88 Methyl (4aS,11aS)-8-(4-fluorophenyl)-11a-methyl-4-(trifluoromethyl)-3,4,4a,5,6,8,11,11a-octahydro-2H-naphtho[1,2-f]indazole-4carboxylate, diastereomer 2: δ 3.52 (dd, J=6.5 Hz, 9.5 Hz, 1H, H₄ₐ), 3.18 (d, J=15.6 Hz, 1H, H₁₁), 2.33-2.42 (m, 2H, H₂, H₃), 2.21 (d, J=15.6 Hz, 1H, H₁₁), 2.03 (m, 1H, H₆).

EXAMPLE 89 Methyl (4aR,11aS)-8-(4-fluorophenyl)-11a-methyl-4-(trifluoromethyl)-3,4,4a,5,6,8,11,11a-octahydro-2H-naphtho[1,2-f]indazole-4-carboxylate, diastereomer 1: δ 3.45 (br d, J=13.5 Hz, 1H, H₄ₐ), 2.86 (d, J=15 Hz, 1H, H₁₁), 2.46 (m, 1H, H₆), 2.39 (d, J=15 Hz, 1H, H₁₁), 2.12-2.33 (m, 3H, H₂, H₃, H₆).

EXAMPLE 90 Methyl (4aR,11aS)-8-(4-fluorophenyl)-11a-methyl-4-(trifluoromethyl)-3,4,4a,5,6,8,11,11a-octahydro-2H-naphtho[1,2-f]indazole-4 carboxylate, diastereomer 2: δ 2.90 (br d, J=12.5 Hz, 1H, H₄ₐ), 2.86 (d, J=15.5 Hz, 1H, H₁₁), 2.55 (d, J=15.5 Hz, 1H, H₁₁), 2.26 (m, 1H, H₆), 2.01-2.20 (m, 3H, H₂, H₃, H₃).

### EXAMPLES 91 AND 92

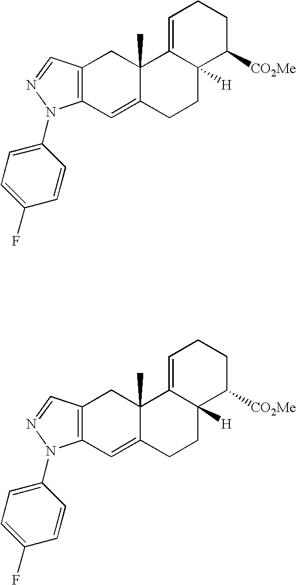

### Methyl (4R,4aR,11aS)-8-(4-fluorophenyl)-11a-methyl-3,4,4a,5 6,8,11,11a-octahydro-2H-naphtho[2-f]indazole-4-carboxylate and methyl (4S,4aS11aS)-8-(4-fluorophenyl)-11a-methyl-3,4,4a,5,6,8,11,11a-octahydro-2H-naphtho[1,2-f]indazole-4-carboxylate

The title compounds were prepared from (4aS)-1-(4-fluorophenyl)-4a-methyl-5-vinyl-4,4a,7,8-tetrahydro-1H-benzo[f]indazole from EXAMPLE 1 Step F and methyl acrylate according to the procedure described in EXAMPLE 82.

Purification by HPLC on Chiralcel OJ, eluting with 5% ethanol-heptane provided two clean diastereomers. Mass spectrum (ESI) 393 (M+1). ¹H NMR (C₆D₆) diagnostic peaks only:

EXAMPLE 91 Methyl (4R,4aR,11aS)-8-(4-fluorophenyl)-11a-methyl-3,4,4a,5,6,8,11,11a-octahydro-2H-naphtho[1,2-f]indazole-4-carboxylate (500 MHz): δ 2.83 (m, 1H, H₄ₐ), 2.72 (d, J=16 Hz, 1H, H₁₁), 2.50 (ddd, J=2.5 Hz, 5.5 Hz, 12.5 Hz, 1H, H₄).

EXAMPLE 92 Methyl (4S,4aS,11aS)-8-(4-fluorophenyl)-11a-methyl-3,4,4a,5,6,8,11,11a-octahydro-2H-naphtho[1,2-f]indazole-4-carboxylate (600 MHz): δ 2.91 (d, J=16 Hz, 1H, H₁₁), 2.76 (m, 1H, H₄ₐ), 2.63 (ddd, J=3 Hz, 6 Hz, 12.5 Hz, 1H, H₄).

### EXAMPLES 93 AND 94

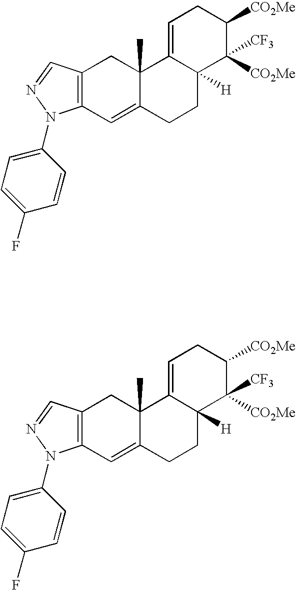

### Dimethyl (3R,4R,4aS,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate and dimethyl (3S,4S,4aR,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate

To a suspension of LiClO₄ (532 mg) in 0.5 mL of Et₂O was added 100 mg of (4aS)-1-(4-fluorophenyl)-4a-methyl-5-vinyl-4,4a,7,8-tetrahydro-1H-benzo[f]indazole from EXAMPLE 1 Step F in 0.5 mL of Et₂O, then 60 mg of trifluoromethylmaleic anhydride. The mixture, which gradually became homogeneous, was stirred at room temperature overnight (20 h), then poured into 5 mL NaHCO₃ and extracted with 3×5 mL EtOAc. The combined organics were washed with brine, dried (Na₂SO₄), and concentrated. The residue was dissolved in 3 mL of 2:1 benzene-MeOH and excess TMSCH₂N₂ was added. The mixture was stirred overnight, then quenched with trifluoroacetic acid and concentrated. Preparative TLC, eluting with 3:1 hexanes-EtOAc, provided 14 mg of dimethyl (3S,4S,4aR,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate, EXAMPLE 94, and 60 mg of a 10 to 1 mixture of dimethyl (3R,4R,4aS,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate and dimethyl (3S,4S,4aR,11aS)-8-(4-fluorophenyl)-11a-methyl-3-(trifluoromethyl)-3,4,4a,5,6,8,11,11a-octahydro-2H-naphtho[1,2-f]indazole-3,4-dicarboxylate. Part of this mixture was used in Example 103 and 104 and the remainder was further purified by HPLC on Chiralcel OJ, eluting with 10% ethanol-heptane to give the title compound of Example 93. Mass spectrum (ESI) 519 (M+1). ¹H NMR (500 MHz) diagnostic peaks only:

EXAMPLE 93 Dimethyl (3R,4R,4aS,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate (CDCl₃): δ 3.08 (dd, J=6 Hz, 9 Hz, 1H, H₃), 2.93 (br t, J=9.5 Hz, 1H, H₄ₐ).

EXAMPLE 94 Dimethyl (3S,4S,4aR,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate (C₆D₆): δ 2.99 (dd, J=6.5 Hz, 11.5 Hz, 1H, H₃), 2.81 (br d, J=11.5 Hz, 1H, H₄ₐ).

### EXAMPLES 95 AND 96

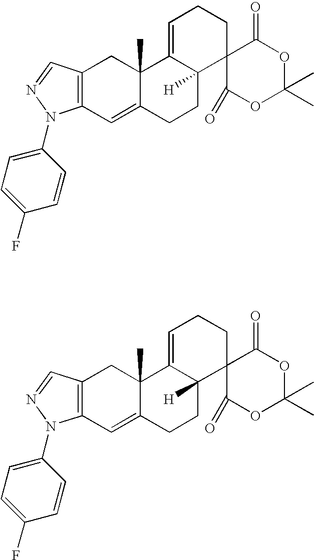

### (4aR,11aS)-8-(4-Fluorophenyl)-11a-methyl-3 4,4a,5,6,8,11,11a-octahydro-2H-naphtho[1,2-f]indazole-4-(2,2-dimethyl-1,3-dioxane-4,6-dione) and (4aS,11aS)-8-(4-fluorophenyl)-11a-methyl-3,4,4a,5,6,8,11,11a-octahydro-2H-naphtho[1,2-f]indazole-4-(2,2-dimethyl-1,3-dioxane-4,6-dione)

To a solution of 100 mg of (4aS)-1-(4-fluorophenyl)-4a-methyl-5-vinyl-4,4a,7,8-tetrahydro-1H-benzo[f]indazole from EXAMPLE 1 Step F, 47 mg of 2,2-dimethyl-1,3-dioxane4,6-dione, and 4 mg of L-proline in 1 μL of CH₃CN was added 32 μmL of a 37% aqueous solution of formaldehyde. The mixture was stirred at room temperature for 1 h; then an additional 5 mg of 2,2-dimethyl-1,3-dioxane-4,6-dione and 5 μL of formaldehyde were added. After 5 min, a white precipitate formed and the mixture was quenched by addition of ice, then filtered, washing solids with cold water. The solid precipitate was dried under vacuum to yield 129 mg of a 10:1 mixture of the title compounds. Recrystallization (2 crops) from t-butyl methyl ether provided 93 mg of (4aR,11aS)-8-(4-fluorophenyl)-11a-methyl-3,4,4a,5,6,8,11,11a-octahydro-2H-naphtho[1,2-f]indazole-4(2,2-dimethyl-1,3-dioxane4,6-one). Preparative TLC of the filtrate, eluting with 1:1 hexanes-EtOAc, provided an additional 18 mg of the major diastereomer, and 10 mg of (4aS,11aS)-8-(4-fluorophenyl)-11a-methyl-3,4,4a,5,6,8,11,11a-octahydro-2H-naphtho[1,2-f]indazole-4-(2,2-dimethyl-1,3-dioxane-4,6-dione). Mass spectrum (ESI) 463 (M+1). ¹H NMR (500 MHz, CDCl₃) diagnostic peaks only:

EXAMPLE 95 (4aR,11aS)-8-(4-fluorophenyl)-11a-methyl-3,4,4a,5,6,8,11,11a-octahydro-2H-naphtho[1,2-f]indazole-4(2,2-dimethyl-1,3-dioxane-4,6-dione): δ 5.86 (m, 1H, H₄ₐ).

EXAMPLE 96(4aS,11aS)-8-(4-fluorophenyl)-11a-methyl-3,4,4a,5,6,8,11,11a-octahydro-2H-naphtho[1,2-f]indazole-4-(2,2-dimethyl-1,3-dioxane4,6-dione): δ 3.33 (br d, J=12.6 Hz, 1H, H₄ₐ).

### EXAMPLES 97 AND 98

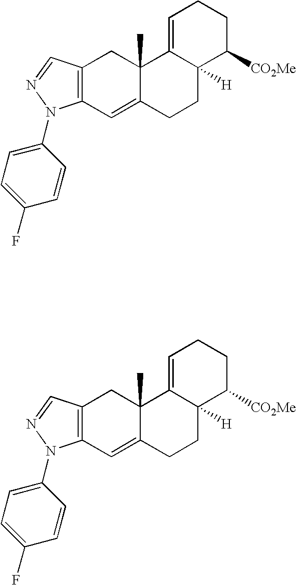

### Methyl (4R,4aR,11aS)-8-(4-fluorophenyl)-11a-methyl-3,4,4a,5,6,8,11,11a-octahydro-2H-naphtho[1,2-f]indazole-4-carboxylate and methyl (4S,4aR,11aS)-8-(4-fluorophenyl)-11a-methyl-3,4,4a,5,6,8,11,11a-octahydro-2H-naphtho[1,2-f]indazole-4-carboxylate

To a solution of 890 mg of (4aR,11aS)-8-(4-fluorophenyl)-11a-methyl-3,4,4a,5,6,8,11,11a-octahydro-2H-naphtho[1,2-f]indazole-4-(2,2-dimethyl-1,3-dioxane-4,6-dione from EXAMPLE 95 in 10 mL of DMSO was added 200 μL of water, then 81 mg of LiOH. The mixture was heated to 120 C. for 4 h, then cooled, poured into 20 mL of 1N HCl and extracted with 3×20 mL of Et₂O. The combined organics were washed with 10 mL of brine, dried (Na₂SO₄), and concentrated. The residue was dissolved in 5 mL of benzene and 0.5 mL of MeOH and treated with 1.5 mL of 2.0 M TMSCH₂N₂, then quenched with trifluoroacetic acid and concentrated. Flash chromatography (Biotage Horizon system, 40S cartridge) with a gradient of 2% EtOAc in hexanes to 100% EtOAc provided 650 mg of the title compounds as a 1.6:1 mixture of diastereomers. Further purification of a 140 mg aliquot of the mixture by HPLC on Chiralpak AD, eluting with 50% isopropanol-heptane, provided 76 mg of methyl (4R,4aR,11aS)-8-(4-fluorophenyl)-11a-methyl-3,4,4a,5,6,8,11,11a-octahydro-2H-naphtho[1,2-f]indazole-4-carboxylate and 46 mg of methyl (4S,4aR,11aS)-8-(4-fluorophenyl)-11a-methyl-3,4,4a,5,6,8,11,11a-octahydro-2H-naphtho[1,2-f]indazole-4-carboxylate. Mass spectrum (ESI) 393 (M+1). ¹H NMR (500 MHz, C₆D₆) diagnostic peaks only:

EXAMPLE 97 Methyl (4R,4aR,11aS)-8-(4-fluorophenyl)-11a-methyl-3,4,4a,5,6,8,11,11a-octahydro-2H-naphtho[1,2-f]indazole-4-carboxylate: see EXAMPLE 91

EXAMPLE 98 Methyl (4S,4aR,11aS)-8-(4-fluorophenyl)-11a-methyl-3,4,4a,5,6,8,11,11a-octahydro-2H-naphtho[1,2-f]indazole-4-carboxylate: δ 3.08 (d, J=16 Hz, 1H, H₁₁), 2.91 (m, 1H, H₄ₐ), 2.31 (m, 1H, H₄).

### EXAMPLES 99 AND 100

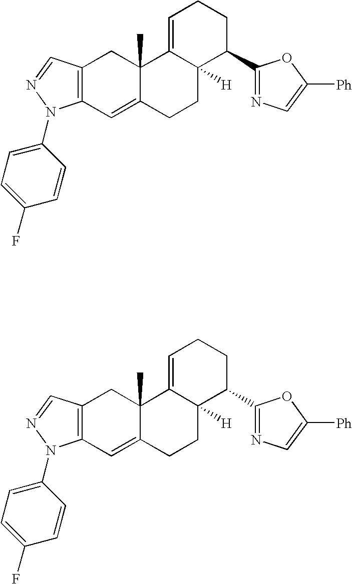

### (4R,4aR,11aS)-8-(4-Fluorophenyl)-11a-methyl-4-(5-phenyl-1,3-oxazol-2-yl)-3,4,4a,5,6,8,11,11a-octahydro-2H-naphtho[1,2-f]indazole and (4S,4aR,11aS)-8-(4-fluorophenyl)-11a-methyl-4-(5-phenyl-1,3-oxazol-2-yl)-3,4,4a,5,6,8,11,11a-octahydro-2H-naphtho[1,2-f]indazole

### Step A: (4aR,11aS)-8-(4-Fluorophenyl)-11a-methyl-3,4,4a,5,6,8,11,11a-octahydro-2H-naphtho[1,2-f]indazole-4-carboxylic acid

To a solution of 25 mg of a ca. 1:1 mixture of methyl (4R,4aR,11aS)-8-(4-fluorophenyl)-11a-methyl-3,4,4a,5,6,8,11,11a-octahydro-2H-naphtho[1,2-f]indazole-4-carboxylate and methyl (4S,4aR,11aS)-8-(4-fluorophenyl)-11a-methyl-3,4,4a,5,6,8,11,11a-octahydro-2H-naphtho[1,2-f]indazole-4-carboxylate (EXAMPLEs 97 and 98 ) in 1.5 mL of 1:1:1 THF—MeOH—H₂O was added LiOH. The mixture was stirred overnight (18 h) at 45° C., then cooled, diluted with 10 mL of 1 N HCl, and extracted with 3×10 mL of EtOAc. The combined organics were dried (Na₂SO₄) and concentrated to yield 21 mg of the title compound as a ca. 1:1 mixture of diastereomers. Mass spectrum (ESI) 379 (M+1).

### Step B: (4aR,11aS)-8-(4-Fluorophenyl)-11a-methyl-3,4,4a,5,6,8,11,11a-octahydro-2H-naphtho[1,2-f]indazole-4-N-(2-oxo-2-phenylethyl)carboxamide

To a 0° C. solution of 45 mg of (4aR,11aS)-8-(4-fluorophenyl)-11a-methyl-3,4,4a,5,6,8,11,11a-octahydro-2H-naphtho[1,2-f]indazole-4-carboxylic acid (a ca. 1:1 mixture of diastereomers, EXAMPLE 99, Step A) in 1 mL of CH₂Cl₂ was added oxalyl chloride (90 μL of a 2.0 M solution in CH₂Cl₂), then DMF (5 μL). The mixture was stirred at 0° C. until gas evolution ceased; then the bath was removed and stirring was continued for 30 min, at which point LC/MS analysis showed no starting material. The mixture was concentrated, then co-concentrated twice with 10 mL toluene, then redissolved in 1 mL of CH₂Cl₂. Aminoacetophenone (22 mg) and Et₃N (36 μL) were added and the mixture was stirred overnight at room temperature. The mixture was diluted with 10 mL of 10% aq NH₄OH, and extracted with 3×10 mL of CH₂Cl₂. The combined organics were washed with 10 mL of brine, dried (Na₂SO₄) and concentrated. Preparative TLC, eluting with 1:1 hexanes-EtOAc provided 40 mg of the title compound as a ca. 1:1 mixture of diastereomers. Mass spectrum (ESI) 496 (M+1).

### Step C: (4R,4aR,11aS)-8-(4-fluorophenyl)-11a-Methyl4(5-phenyl-1,3-oxazol-2-yl)-3,4,4a,5,6,8,11,11a-octahydro-2H-naphtho[1,2-f]indazole and (4S,4aR,11aS)-8-(4-fluorophenyl)-11a-methyl4-(5-phenyl-1,3-oxazol-2-yl)-3,4,4a,5,6,8,11,11a-octahydro-2H-naphtho[2-f]indazole

A solution of 38 mg of (4aR,11aS)-8-(4-fluorophenyl)-11a-methyl-3,4,4a,5,6,8,11,11a-octahydro-2H-naphtho[1,2-f]indazole-4-N-(2-oxo-2-phenylethyl)carboxamide (a ca. 1:1 mixture of diastereomers, EXAMPLE 99, Step B) in 1 mL of phosphorus oxychloride was heated to reflux and stirred at this temperature for 30 min, then quenched by careful addition of ice. The mixture was basified with conc. NH₄OH (exothermic), diluted with 5 mL of H₂O and extracted with 3×10 mL of CH₂Cl₂. The combined organics were washed with 10 mL of brine, dried (Na₂SO₄) and concentrated. Preparative TLC, eluting with 2:1 hexanes-EtOAc provided 10 mg of each of the title compounds. Mass spectrum (ESI) 496 (M+1). ¹H NMR (600 MHz, C₆D₆) diagnostic peaks only:

EXAMPLE 99 (4R,4aR,11aS)-8-(4-fluorophenyl)-11a-methyl-4-(5-phenyl-1,3-oxazol-2-yl)-3,4,4a,5,6,8,11,11a-octahydro-2H-naphtho[1,2-f]indazole: δ 3.06 (m, H₄), 3.00 (m, 1H, H₄ₐ), 2.78 (d, J=16 Hz, 1 H, H₁₁), 2.19 (d, J=16 Hz, 1 H, H₁₁), 2.12 (m, 1 H, H₃), 1.86-2.06 (m, 4 H, H₂, H₂, H₃, H₆).

EXAMPLE 100 (4S,4aR,11aS)-8-(4-fluorophenyl)-11a-methyl-4-(5-phenyl-1,3-oxazol-2-yl)-3,4,4a,5,6,8,11,11a-octahydro-2H-naphtho[1,2-f]indazole: δ 3.23 (d, J=16 Hz, 1 H, H₁₁), 3.15 (m, H₄ₐ), 2.85 (m, 1H, H₄), 2.30(d, J=16 Hz, 1 H, H₁₁), 2.12 (m, 1 H, H₃), 1.86-2.16 (m, 5 H, H₂, H₂, H₃, H₆, H₆).

### EXAMPLES 101 AND 102

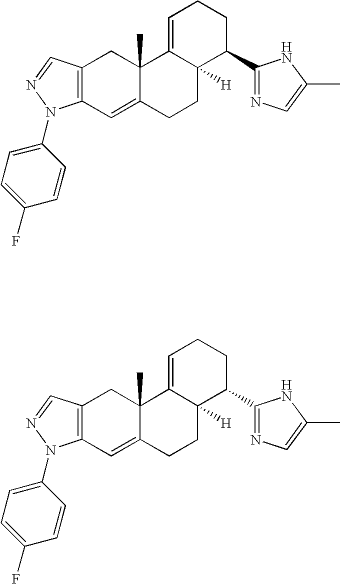

### (4R,4aR,11aS)-8-(4-fluorophenyl)-11a-Methyl-4-(5-methyl-1H-imidazol-2-yl)-3,4,4a,5,6,8,11,11a-octahydro-2H-naphtho[1,2-f]indazole and (4S,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole

### Step A: (4aR 11aS)-8-(4-Fluorophenyl)-11a-methyl-3,4,4a,5,6,8,11,11a-octahydro-2H-naphtho[1,2-f]indazole-4-carbaldehyde

To a 0° C. solution of 105 mg of a ca. 1:1 mixture of methyl (4R,4aR,11aS)-8-(4-fluorophenyl)-11a-methyl-3,4,4a,5,6,8,11,11a-octahydro-2H-naphtho[1,2-f]indazole-4-carboxylate and methyl (4S,4aR, 11aS)-8-(4-fluorophenyl)-11a-methyl-3,4,4a,5,6,8,11,11a-octahydro-2H-naphtho[1,2-f]indazole-4-carboxylate (EXAMPLES 97 and 98 ) in 3 mL of THF was added 0.3 mL of a 1.0 M solution of LiAlH₄ in THF dropwise. The mixture was stirred 15 min at 0° C.; then the bath was removed and the mixture was stirred 30 min while warming to room temperature, at which point TLC showed no starting material. To the mixture was added 11 μL of H₂O, then 11 μL of 15% aq NaOH, then 33 μL of H₂O. The mixture was stirred 30 min, then filtered and concentrated. The residue was dissolved in 1 mL of CH₂Cl₂. To 0.31 mL of a 2.0 M solution of oxalyl chloride in 2 mL of CH₂Cl₂ at −78° C. was added 36 μL of DMSO. The mixture was stirred 5 min at −78° C.; then the solution of alcohol in 1 mL CH₂Cl₂ was added dropwise and stirring was continued for 15 min. Et₃N (0.14 mL) was added and the mixture was stirred 10 min at −78° C., then allowed to warm to room temperature. The mixture was poured into 10 mL of saturated NaHCO₃ and extracted with 15 mL of EtOAc. The organic extract was washed with 10 mL each of 1 N NaHSO₄, saturated NaHCO₃, and brine, then dried (Na₂SO₄) and concentrated to yield 67 mg of the title compound as a ca. 1:1 mixture of diastereomers. Mass spectrum (ESI) 363 (M+1).

### Step B: (4R,4aR,11aS)-8-(4-fluorophenyl)-11a-Methyl-4-(5-methyl-1H-imidazol-2-yl)-3,4,4a,5,6,8,11,11a-octahydro-2H-naphtho[1,2-f]indazole and (4S,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole

To a 0° C. solution of 87 mg of (4aR,11aS)-8-(4-fluorophenyl)-11a-methyl-3,4,4a,5,6,8,11,11a-octahydro-2H-naphtho[1,2-f]indazole-4-carbaldehyde (a ca. 1:1 mixture of diastereomers), from Step A in 3 mL of MeOH was added ammonium acetate (185 mg) and methyl glyoxal (0.13 mL of a 40% aqueous solution). The mixture was heated to reflux and stirred at this temperature overnight. The mixture was cooled and concentrated, then dissolved in minimal CH₂Cl₂—MeOH and purified by preparative TLC, eluting with 9:1 CH₂Cl₂—MeOH to provide 14 mg of (4R,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole and 12 mg of (4S,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole. Mass spectrum (ESI) 415 (M+1). ¹H NMR (500 MHz, CD₃OD) diagnostic peaks only:

EXAMPLE 101 (4R,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole: δ 3.13 (m, 1H, H₄), 2.97 (d, J=15.5 Hz, 1 H, H₁₁), 2.80 (m, 1H, H₄ₐ), 2.43 (d, J=15.5 Hz, 1 H, H₁₁).

EXAMPLE 102 (4S,4aR,11aS)-8-(4-fluorophenyl)-11a-methyl-4-(5-methyl-1H-imidazol-2-yl)-3,4,4a,5,6,8,11,11a-octahydro-2H-naphtho[1,2-f]indazole: δ 2.92 (d, J=16 Hz, 1 H, H₁₁), 2.70-2.80 (m, 2H, H₄ₐ, H₄), 2.51 (d, J=16 Hz, 1 H, H₁₁).

### EXAMPLES103 AND 104

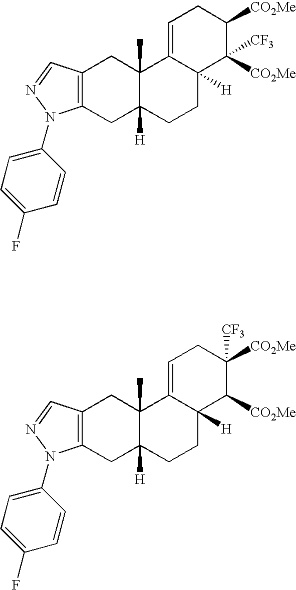

### Dimethyl (3R,4R,4aS,6aR,11aS)-8-(4-fluorophenyl)-11a-methyl-4-(trifluoromethyl)-3,4,4a,5,6,6a,7,8,11,11a-decahydro-2H-naphtho[1,2-f]indazole-3,4-dicarboxylate and dimethyl (3S,4S,4aR,6aR,11aS)-8-(4-fluorophenyl)-11a-methyl-3-(trifluoromethyl)-3,4,4a,5,6,6a,7,8,11,11a-decahydro-2H-naphtho[1,2-f]indazole-3,4-dicarboxylate

To a solution of 20 mg of a ca. 10:1 mix of dimethyl (3R,4R,4aS,11aS)-8-(4-fluorophenyl)-11a-methyl-4-(trifluoromethyl)-3,4,4a,5,6,8,11,11a-octahydro-2H-naphtho[1,2-f]indazole-3,4-dicarboxylate and dimethyl (3S,4S,4aR,11aS)-8-(4-fluorophenyl)-11a-methyl-3-(trifluoromethyl)-3,4,4a,5,6,8,11,11a-octahydro-2H-naphtho[1,2-f]indazole-3,4-dicarboxylate (see EXAMPLE 93) in 1 mL of EtOAc was added 10 Mg of PtO₂ (Adam's catalyst). The mixture was flushed with N₂, then flushed with H₂ and stirred 3 h under an H₂ balloon, at which point LC/MS analysis showed no starting material. The mixture was filtered through Celite, washing liberally with MeOH, then concentrated. Preparative TLC, eluting with 3:1 hexanes-EtOAc, provided 15.9 mg of dimethyl (3R,4R,4aS,6aR,11aS)-8-(4-fluorophenyl)-11a-methyl4-(trifluoromethyl)-3,4,4a,5,6,6a,7,8,11,11a-decahydro-2H-naphtho[1,2-f]indazole-3,4-dicarboxylate and 1.2 mg of dimethyl (3S,4S,4aR,6aR,11aS)-8-(4-fluorophenyl)-11a-methyl-3-(trifluoromethyl)-3,4,4a,5,6,6a,7,8,11,11a-decahydro-2H-naphtho[1,2-f]indazole-3,4-dicarboxylate. Mass spectrum (ESI) 521 (M+1). ¹H NMR (500 MHz, CDCl₃) diagnostic peaks only:

EXAMPLE 103 Dimethyl (3R,4R,4aS,6aR,11aS)-8-(4-fluorophenyl)-11a-methyl-4-(trifluoromethyl)-3,4,4a,5,6,6a,7,8,11,11a-decahydro-2H-naphtho[1,2-f]indazole-3,4-dicarboxylate: 3.04 (dd, J=6 Hz, 17 Hz, 1H, H₇), 2.78-2.95 (m, 4H, H₂, H₃, H₄ₐ, H₁₁), 2.34-2.46 (m, 2H, H₇, H₂), 1.68 (m, 1H, H₆ₐ).

EXAMPLE 104 Dimethyl (3S,4S,4aR,6aR,11aS)-8-(4-fluorophenyl)-11a-methyl-3-(trifluoromethyl)-3,4,4a,5,6,6a,7,8,11,11a-decahydro-2H-naphtho[1,2-f]indazole-3,4-dicarboxylate: δ 3.59 (d, J=6.5 Hz, 1H, H₄), 3.27 (br d, J=19 Hz, 1H, H₂), 3.06 (dd, J=6.5 Hz, 17 Hz, 1H, H₇), 2.75-2.85 (m, 2H, H₄ₐ, H₁₁), 2.60 (dd, J5.5 Hz, 19.5 Hz, 1H, H₂), 2.41 (d, J=16.5 Hz, 1H, H₇), 1.69 (m, 1H, H₆ₐ)

### EXAMPLE 105

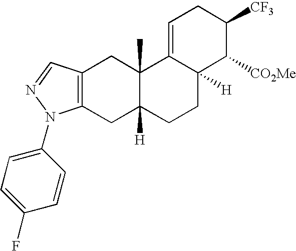

### Methyl (3R,4R,4aR,6aR,11aS)-8-(4-fluorophenyl)-11a-methyl-3-(trifluoromethyl)-3,4,4a5,6,6a,7,8,11,11a-decahydro-2H-naphtho[1,2-f]indazole-4-carboxylate

The title compound was prepared from methyl (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylate (EXAMPLE 1) according to the procedure described in EXAMPLE 103. Final purification by HPLC on Chiralpak AD, eluting with 10% ethanol-heptane provided the title compound. Mass spectrum (ESI) 477 (M+1). ¹H NMR (500 MHz, CDCl₃) diagnostic peaks only: δ 3.04 (dd, J=6 Hz, 17 Hz, 1H, H₇), 2.93 (d, J=16 Hz, 1H, H₁₁), 2.73 (m, 1H, H₄ₐ), 2.57 (m, 1H, H₃), 2.36-2.46 (m, 2H, H₇, H₂), 2.33 (dd, J=10.5 Hz, 11.5 Hz, 1H, H₄), 1.66 (m, 1H, H₆a).

### EXAMPLE 109

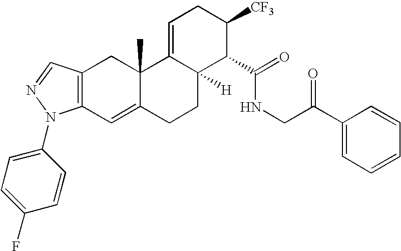

### (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-(2-oxo-2-phenylethyl)carboxamide

A solution of (3R,4R,4aR,11aS)-8-(4-fluorophenyl)-11a-methyl-3-(trifluoromethyl)-3,4,4a,5,6,8,11,11a-octahydro-2H-naphtho[1,2-f]indazole-4-carboxylic acid (40.8 mg, 0.091 mmol) in CH₂Cl₂ (1.0 mL) was cooled down to 0° C., and to this was added oxalyl chloride (70 μL, 2.0 M, 0.137 mmol) followed by 1 drop of DMF via pipet. The reaction mixture was stirred for 2 h while slowly warming up to room temperature. The solvent was removed under reduced pressure, and the residue was co-evaporated two times with toluene. After drying under high vacuum, the acid chloride was dissolved in CH₂Cl₂ (1.0 mL), and to this was added 2-aminoacetophenone hydrochloride (18 mg, 0.1 mmol) followed by Et₃N (28 μL, 0.2 mmol). The reaction mixture was stirred for 2 h, then 10% aqueous NH₄OH was added. The aqueous layer was extracted with CH₂Cl₂, and combined extracts were washed with brine and dried over Na₂SO₄. The crude material was purified by preparative TLC eluting with 1:2 acetone:hexane to obtain 42 mg of the title compound.

¹H NMR (CDCl₃, 500MHz): δ 7.97 (d, J=7.6 Hz, 2H); 7.62-7.39 (m, 6H); 7.14 (t, J=8.6 Hz, 2H); 6.82 (1 NH); 6.16 (s, 1H); 5.73 (d, J=6.0 Hz, 1H); 4.91 (dd, J=5, 19.9 Hz, 1H); 4.68 (dd, J=3.5, 19.9 Hz, 1H); 2.86 (m, 1H); 2.87 (d, J=15.8 Hz, 1H); 2.71 (m, 1H); 2.5 (d, J=15.8 Hz, 1H); 2.44-2.21 (m, 5H); 2.01 (m, 1H); 1.5 (m, 1H); 1.22 (s, 3H). Mass spectrum (ESI): 564.2 (M+1).

The following examples were synthesized by procedures analogous to that described in EXAMPLE 109:

| Compound | Molecular Structure | LCMS (M + 1)⁺ |
|---|---|---|
| 110 | 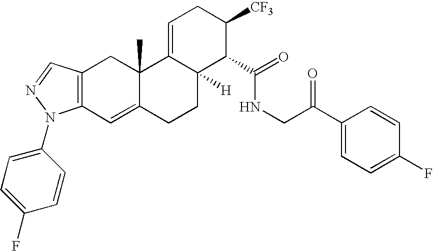 | 582 |
| 111 | 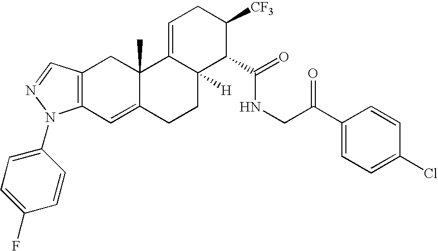 | 598 |
| 112 | 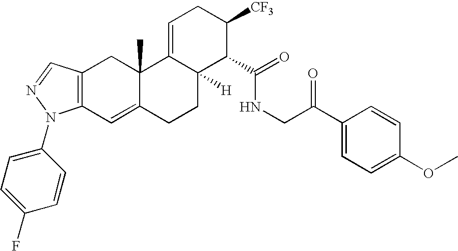 | 594 |
| 113 | 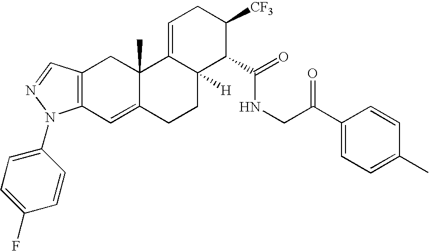 | 578 |

### EXAMPLE 118

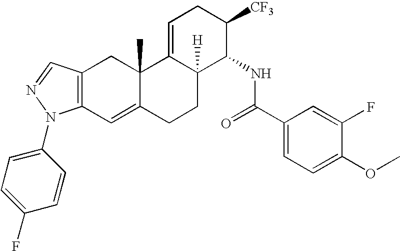

### 3-fluoro-N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-4-methoxybenzamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 570 (M+1).

### EXAMPLE 119

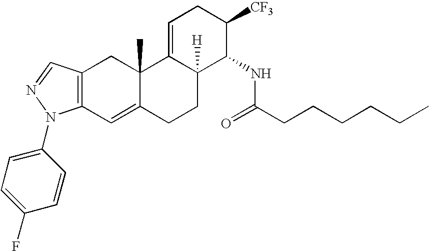

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]heptanamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 530 (M+1).

### EXAMPLE 120

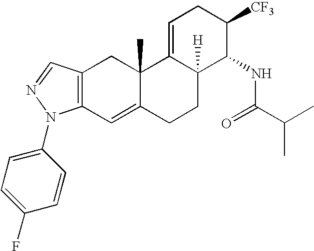

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-2-methylpropanamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 488 (M+1).

### EXAMPLE 121

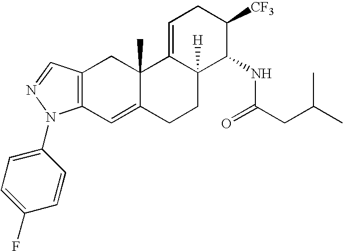

### N-[(3R,4R,4aS,11aS)-8-(4-fluorophenyl)-11a-methyl-3-(trifluoromethyl)-3,4,4a5,6,8,11,11a-octahydro-2H-naphtho[1,2-f]indazol-4-yl]-3-methylbutanamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

The product was purified by flash column chromatography on silica gel eluting with 1%-100% methanol in dichloromethane.

Mass spectrum (ESI) 502 (M+1).

### EXAMPLE 122

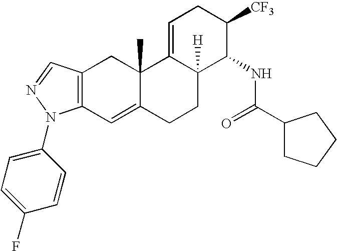

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]cyclopentanecarboxamide

The title compound was prepared from (3R,4R,4aS,11a)8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

The product was purified by preparative thin-layer chromatography, eluting with 97.5:2.5 dichloromethane-methanol.

Mass spectrum (ESI) 514 (N+1).

### EXAMPLE 123

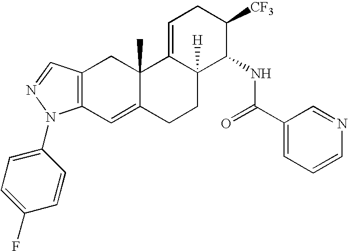

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]nicotinamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 523 (M+1).

### EXAMPLE 124

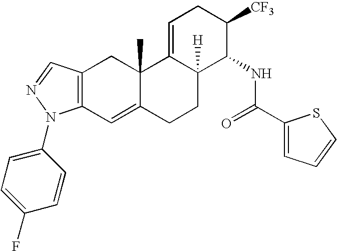

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]thiophene-2-carboxamide

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 528 (M+1).

### EXAMPLE 125

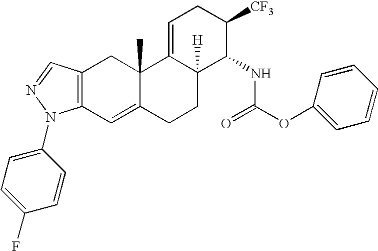

### Phenyl [(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]carbamate

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

Mass spectrum (ESI) 538 (M+1).

### EXAMPLE 126

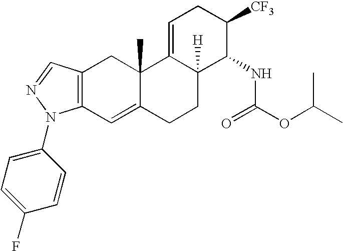

### Isopropyl [(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]carbamate

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

The product was purified by preparative thin-layer chromatography, eluting with 97.5:2.5 dichloromethane-methanol.

Mass spectrum (ESI) 504 (M+1).

### EXAMPLE 127

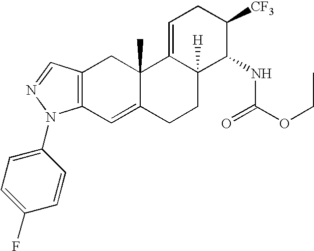

### Ethyl [(3R,4R,4aS11aS)-8-(4-fluorophenyl)-11a-methyl-3-(trifluoromethyl)-3,4,4a,5,6,8,11,11a-octahydro-2H-naphtho[1,2-f]indazol-4-yl]carbamate

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

The product was purified by preparative thin-layer chromatography, eluting with 97.5:2.5 dichloromethane-methanol.

Mass spectrum (ESI) 490 (M+1).

### EXAMPLE 128

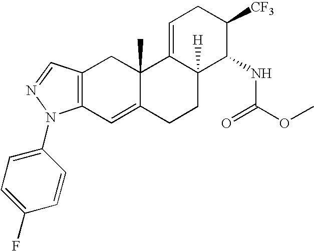

### methyl [(3R,4R,4aS,11aS)-8-(4-fluorophenyl)-11a-methyl-3-(trifluoromethyl)-3,4,4a5,6,8,11,11a-octahydro-2H-naphtho[1,2-f]indazol-4-yl]carbamate

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 63.

The product was purified by preparative thin-layer chromatography, eluting with 75:25 dichloromethane-methanol.

Mass spectrum (ESI) 476 (M+1).

### EXAMPLE 129

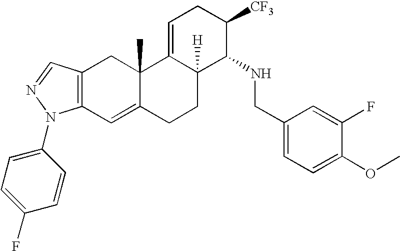

### (3R,4R,4aS,11aS)-N-(3-fluoro-4-methoxybenzyl)-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 556 (M+1).

### EXAMPLE 130

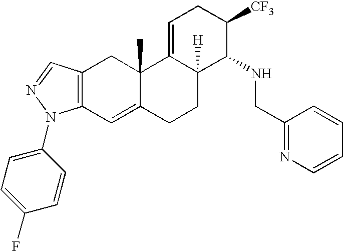

### (3R,4R,4aS,11aS)-8-(4-fluorophenyl)-11a-methyl-N-(pyridin-2-ylmethyl)-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 509 (M+1).

### EXAMPLE 131

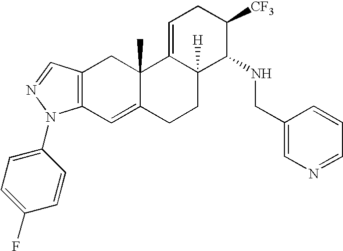

### (3R,4R,4aS,11aS)-8-(4-fluorophenyl)-11a-methyl-N-(pyridin-3-ylmethyl)-3-(trifluoromethyl)-3,4,4a,5,6,8,11,11a-octahydro-2H-naphtho[2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 509 (M+1).

### EXAMPLE 132

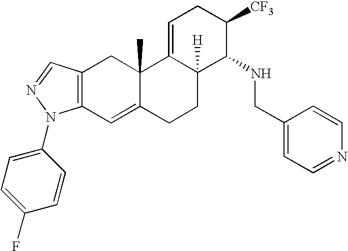

### (3R,4R,4aS,11aS)-8-(4-fluorophenyl)-11a-methyl-N-(pyridin-4-ylmethyl)-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine EXAMPLE 62) by a procedure analogous to that described in EXAMPLE 70.

The product was purified by preparative thin-layer chromatography, eluting with 95:5 dichloromethane-methanol.

Mass spectrum (ESI) 509 (M+1).

### EXAMPLE 133

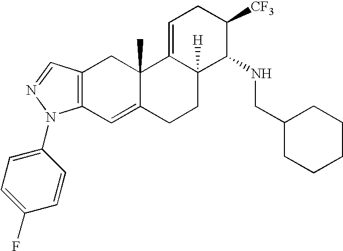

### (3R,4R,4aS,11aS)-N-(cyclohexylmethyl)-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 514 (M+1).

### EXAMPLE 134

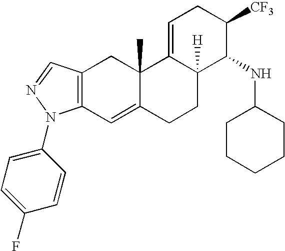

### (3R,4R,4aS,11aS)-N-cyclohexyl-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 500 (M+1).

### EXAMPLE 135

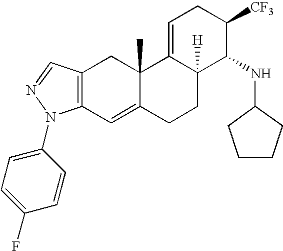

### (3R,4R,4aS,11aS)-N-cyclopentyl-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 486 (M+1).

### EXAMPLE 136

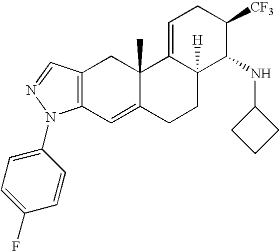

### (3R,4R,4aS,11aS)-N-cyclobutyl-8-(4-fluorophenyl)-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 472 (M+1).

### EXAMPLE 137

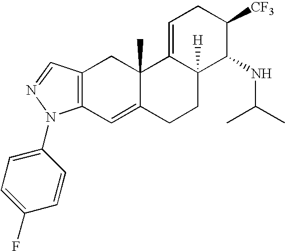

### (3R,4R,4aS 11aS)-8-(4-fluorophenyl)-N-isopropyl-11a-methyl-3-(trifluoromethyl)-3,4,4a,5,6,8,11,11a-octahydro-2H-naphtho[1,2-f]indazol-4-amine

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 70.

The product was purified by preparative thin-layer chromatography, eluting with 97.5:2.5 dichloromethane-methanol.

Mass spectrum (ESI) 460 (M+1).

### EXAMPLE 138

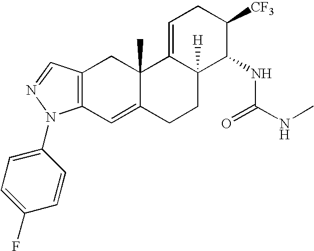

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-N′-methylurea

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine EXAMPLE 62) by a procedure analogous to that described in EXAMPLE 78.

Mass spectrum (ESI) 475 (M+1).

### EXAMPLE 139

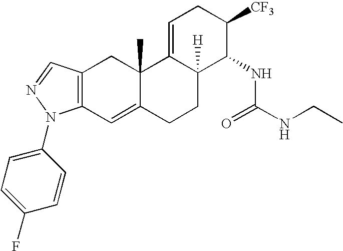

### N-ethyl-N′-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]urea

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 78.

Mass spectrum (ESI) 489 (M+1).

### EXAMPLE 140

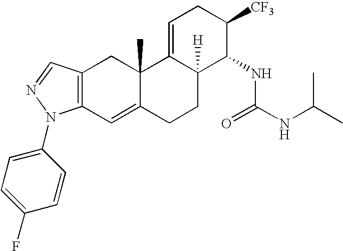

### N-[(3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-yl]-N′-isopropylurea

The title compound was prepared from (3R,4R,4aS,11aS)-8-(4-fluorophenyl)-11a-methyl-3-(trifluoromethyl)-3,4,4a,5,6,8,11,11a-octahydro-2H-naphtho[1,2-f]indazol-4-amine (EXAMPLE 62) by a procedure analogous to that described in EXAMPLE 78.

Mass spectrum (ESI) 503 (M+1).

### BIOLOGICAL ASSAYS

The activity of the compounds of the present invention as modulators of the glucocorticoid receptor can be evaluated using the following assays:

Ligand Binding Assays

For the hGRI ligand binding assay, cytosols were prepared from recombinant baculovirus expressed receptors. Frozen cell pellets were dounce homogenized in ice cold KPO₄ buffer (10 mM KPO₄, 20 mM sodium molybdate, 1 mM EDTA, 5 mM DTT and complete protease inhibitor tablets from Boehringer Mannheim) with a “B” plunger. The homogenates were centrifuged at 35,000×g for 1 h at 4° C. in a JA-20 rotor. The IC₅₀s were determined by incubating the cytosols at a final concentration of 2.5nM [1,2,4,6,7-³H] Dexamethasone in the presence of increasing concentrations (10-11 to 10-6) of cold dexamethasone or the ligands at 4° C. for 24 h. Bound and free were separated by a gel filtration assay, (Geissler et al, personal communication). Half of the reaction was added to a gel filtration plate (MILLIPORE) containing sephadex G-25 beads that was previously equilibrated with KPO4 buffer containing 1 mg/ml BSA and centrifuged at 1000×g for 5 min. The reaction plate was centrifuged at 1000×g for 5 min. and the reactions were collected in a second 96-well plate and scintillation cocktail was added and counted in (Wallac) double coincidence beta counter. The IC₅₀s were calculated using a 4-parameter fit program.

The IC₅₀ of representative compounds of the invention are in the range of 1 nM to 3.0 μM.

1. A compound represented by Formula I 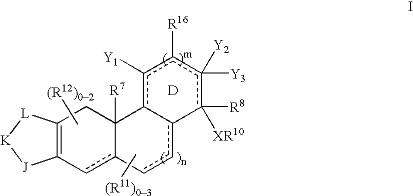
or a pharmaceutically acceptable salt or hydrate thereof, wherein:
n and m are each independently 0, 1 or 2;
J is selected from NR¹ or C(R¹)(R²);
K is selected from NR³ or C(R³)(R⁴);
L is selected from NR⁵ or C(R⁵)(R⁶);
X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);
k is 0, 1 or 2;
R¹ and R¹⁰ are each independently selected from the group consisting of:
(1) C₁₋₆alkyl,
(2) C₂₋₆alkenyl,
(3) C₂₋₆akynyl,
(4) C₃₋₆cycloalkyl,
(5) C₁₋₆alkoxy,
(6) C₁₋₆alkyl-S(O)ₖ—, wherein k is 0, 1 or 2,
(7) aryl,
(8) aryl C₁₋₆alkyl,
(9) HET,
(10) —C₁₋₆alkyl-HET,
(11) aryloxy,
(12) aroyloxy,
(13) aryl C₂₋₆alkenyl,
(14) aryl C₂₋₆alkynyl,
(15) hydrogen,
(16) hydroxyl and
(17) cyano
wherein items (1) to (6) above and the alkyl portions of items (8) and (10) above and the alkenyl portion of item (13) above and the alkynyl portion of item (14) above are optionally substituted from one up to the maximum number of substitutable positions with a substituent independently selected from the group consisting of: halo, oxo, OR¹³, N(R¹⁴)₂, C₃₋₆cycloalkyl and C₁₋₆alkyl-S(O)ₖ—, wherein k is 0, 1 or 2, and
wherein items (7), (9), (11) and (12) above and aryl portion of items (8), (13) and (14) above and the HET portion of item (10) above are optionally substituted from one up to the maximum number of substitutable positions with a substituent independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy,
(o) CN and
(p) C₃₋₆cycloalkyl,
wherein items (d) to (g) and (p) above and the alkyl portions of item (k) above are optionally substituted from one up to the maximum number of substitutable positions with a substituent independently selected from the group consisting of: halo, OR¹³ and N(R¹⁴)₂, and
wherein items (h), (i), (j), (l) and (m) above and the aryl portions of items (k) and (n) above are optionally substituted from one up to the maximum number of substitutable positions with a substituent independently selected from the group consisting of: halo, OR¹³ and C₁₋₄alkyl,
R², R³, R⁴, R⁵ and R⁶ are each independently selected from the group consisting of:
(1) hydrogen,
(2) halo,
(3) C₁₋₆alkyl,
(4) C₂₋₆alkenyl,
(5) C₂₋₆akynyl,
(6) C₃₋₆cycloalkyl,
(7) C₁₋₆alkoxy,
(8) C₁₋₆alkyl-S(O)ₖ—, wherein k is 0, 1 or 2,
(9) aryl,
(10) aryl C₁₋₆alkyl,
(11) HET and
(12) —C₁₋₆alkyl-HET,
wherein items (3) to (8) above and the alkyl portions of items (10) and (12) above are optionally substituted from one up to the maximum number of substitutable positions with a substituent independently selected from the group consisting of: halo, OR¹³, N(R¹⁴)₂ and C₁₋₆alkyl-S(O)ₖ—, wherein k is 0, 1 or 2; and
wherein items (9) and (11) and the aryl portion of items (10) and the HET portion of item (12) are optionally substituted from one up to the maximum number of substituable positions with a substituent independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl and
(g) C₁₋₆alkyl-S(O)ₖ—, wherein k is 0, 1 or 2,
wherein items (d) to (g) above are optionally substituted with from one up to the maximum number of substitutable positions with a substituent independently selected from the group consisting of: halo, OR¹³ and N(R¹⁴)₂,
or R¹ and R³ or R³ and R⁵ may be joined together to form a double bond;
R⁷ is selected from the group consisting of:
(1) hydrogen,
(2) OR¹³,
(3) C₁₋₄alkyl,
(4) aryl and
(5) aryl C₁₋₄alkyl,
wherein item (3) above and the alkyl portion of item (5) above are optionally substituted with from one up to the maximum number of substitutable positions with a substituent independently selected from the group consisting of: halo, OR¹³ and N(R¹⁴)₂, and
wherein item (4) above and the aryl portion of item (5) above are optionally substituted with from one up to the maximum number of substitutable positions with a substituent independently selected from the group consisting of:
(a) halo,
(b) OR¹³,
(c) N(R¹⁴)₂,
(d) C₁₋₆alkyl,
(e) C₂₋₆alkenyl and
(f) C₂₋₆akynyl,
wherein items (d) to (f) above are optionally substituted with from one up to the maximum number of substitutable positions with a substituent independently selected from the group consisting of: halo, OR¹³ and N(R¹⁴)₂;
each Y₁, Y₂ and Y₃ are independently selected from the group consisting of:
(1) hydrogen,
(2) —O—R⁹,
(3) —S(O)ₖ—R⁹, wherein k is 0, 1 or 2,
(4) —C—W—R9, wherein W is 0 or S(O)k,
(5) —N(R¹⁵)₂,
(6) —S(O)ₖ—N(R¹⁵)₂,
(7) —N(R¹⁵)—S(O)k—N(R¹⁵)₂,
(8) NO₂,
(9) —C(O)—R¹⁵,
(10) —C(O)O—R¹⁵,
(11) —CN,
(12) halo,
(13) —O—S(O)ₖ—R¹⁵ and
(14) C₁₋₄alkyl, optionally substituted with from 1 to 6 halo groups,
with the proviso that when Y₂ is hydrogen, Y₃ is —C(O)—R¹⁵, R¹⁵ is C₁₋₆alkyl and X is —C(O) then R¹⁰ is not C₁₋₆alkyl, and
with the proviso that when Y₂ is —C(O)—R¹⁵, Y₃ is hydrogen, R¹⁵ is C₁₋₆alkyl and X is —C(O) then R¹⁰ is not C₁₋₆alkyl, and
with the proviso that when Y₂ and Y₃ are both hydrogen, X is a bond and R¹⁰ is HET, then said HET is defined as a 5-membered aromatic or non-aromatic monocyclic ring containing 1-3 heteroatoms selected from O, S and N,
R⁸ is selected from the group consisting of: hydrogen, C₁₋₆alkyl, C₁₋₆alkoxy, —C₁₋₆alkyl-C(O)OH and —C₁₋₆alkyl-C(O)O—C₁₋₆alkyl, wherein the C₁₋₆alkyl portion is optionally mono, di or tri substituted with halo; or where R⁸ and
—XR¹⁰ together with the carbon atom to which they are attached form the spiro group: 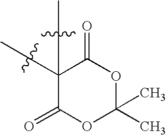
R⁹ is selected from the group consisting of: hydrogen, C₁₋₁₂alkyl and aryl, wherein C₁₋₁₂alkyl and aryl are optionally substituted from one up to the maximum number of substituents with halo;
each R¹¹, R¹² and R¹⁶ is independently selected from the group consisting of:
(1) hydrogen,
(2) halo,
(3) C₁₋₆alkyl,
(4) C₂₋₆alkenyl,
(5) C₁₋₆alkoxy and
(6) hydroxy,
wherein items (3) to (5) above are optionally substituted from one up to the maximum number of substitutable positions with a substituent independently selected from the group consisting of: halo, OR¹², N(R¹³)₂ and C₁₋₆alkyl-S(O)ₖ—, wherein k is 0, 1 or 2,
or R¹⁶ may additionally be hydrogen;
each R¹³ and R¹⁴ is independently selected from the group consisting of hydrogen and C₁₋₄alkyl, optionally substituted from one up to the maximum number of substitutable positions with halo; and
each R¹⁵ is independently selected from the group consisting of: hydrogen, C₁₋₆alkyl, aryl and C₁₋₁₂alkoxycarbonyl, wherein said C₁₋₆alkyl and C₁₋₁₂alkoxycarbonyl are optionally substituted from one up to the maximum number of substituable positions with halo and said aryl is optionally substituted from one up to the maximum number of substituable positions with halo and C₁₋₄alkyl, optionally substituted with 1-3 halo groups.

2. A compound according to claim 1 wherein:
J is NR¹;
K is NR³;
L is C(R⁵)(R⁶); and
R³ and R⁵ are joined together to form a double bond.

3. A compound according to claim 1 of Formula Ia:

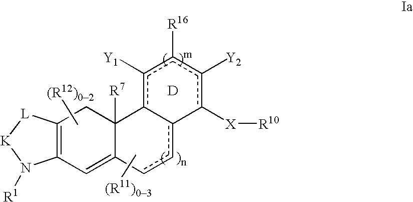

4. A compound according to claim 1 wherein R¹ is phenyl or pyridyl said phenyl or pyridyl or optionally mono or di-substituted with a substituent independently selected from the group consisting of:
(a) halo,
(b) OCH₃,
(d) CH₃,
(e) CN.

5.-10. (canceled)

11. A compound according to claim 1 wherein
X is a bond, —C(O), —N(R¹⁴)—, —N(R¹⁴)—C(O)—, —C(O)—N(R¹⁴)—, —N(R¹⁴)—C(O)—NH—;
Y₁ is hydrogen;
R¹ is phenyl, optionally mono or di-substituted with halo;
R⁷ is methyl.
R¹¹ is hydrogen;
R¹² is hydrogen;
R¹⁴ is hydrogen or methyl;
R¹⁶ is hydrogen; and
R¹⁰ are each independently selected from the group consisting of:
(1) C₁₋₄alkyl,
(2) C₂₋₄alkenyl,
(3) C₂₋₄akynyl,
(4) C₃₋₆cycloalkyl,
(5) C₁₋₄alkoxy,
(6) aryl,
(7) aryl C₁₋₄alkyl,
(8) HET,
(9) —C₁₋₄alkyl-HET,
(10) aryloxy,
(11) aroyloxy,
(12) aryl C₂₋₄alkenyl,
(13) aryl C₂₋₆alkynyl,
wherein items (1) to (5) above and the alkyl portions of items (7) and (9) above and the alkenyl portion of item (12) above and the alkynyl portion of item (13) above are optionally substituted with from one to three substituents independently selected from the group consisting of: halo, OR¹³, N(R¹⁴)₂, C₃₋₆cycloalkyl and C₁₋₆alkyl-S(O)ₖ—, wherein k is 0, 1 or 2, and
wherein items (6), (8), (10) and (11) above and aryl portion of items (7), (12) and (13) above and the HET portion of item (9) above are optionally substituted with from one to three substituents independently selected from the group consisting of:
(a) halo,
(b) OR¹³
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
wherein items (d) to (f) above and the alkyl portions of item (i) above are optionally substituted from with one to three substituents independently selected from the group consisting of: halo, OR¹³ and N(R¹⁴)₂, and
wherein items (g), (h), (j) and (k) above and the aryl portions of items (i) and (l) above are optionally substituted with from one to three substituents independently selected from the group consisting of: halo, OR¹³ and C₁₋₄alkyl,

12. A compound according to claim 1 of Formula Ib 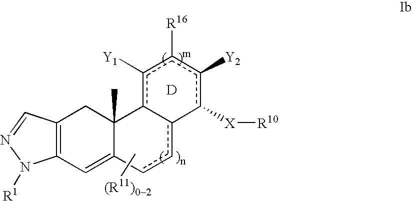
wherein:
m is 0 or 1,
n is 0 or 1,
R¹ is phenyl, optionally mono or di-substituted with halo;
R¹⁰ are each independently selected from the group consisting of:
(1) C₁₋₆alkyl,
(2) C₂₋₆alkenyl,
(3) C₂₋₆akynyl,
(4) C₃₋₆cycloalkyl,
(5) C₁₋₆alkoxy,
(6) C₁₋₆alkyl-S(O)ₖ—, wherein k is 0, 1 or 2,
(7) aryl,
(8) aryl C₁₋₆alkyl,
(9) HET,
(10) —C₁₋₆alkyl-HET,
(11) aryloxy,
(12) aroyloxy,
(13) aryl C₂₋₆alkenyl,
(14) aryl C₂₋₆alkynyl,
(15) hydrogen, and
(16) hydroxy
wherein items (1) to (6) above and the alkyl portions of items (8) and (10) above and the alkenyl portion of item (13) above and the alkynyl portion of item (14) above are optionally substituted with from one to three substituents independently selected from the group consisting of: halo, OR¹³, N(R¹⁴)₂, C₃₋₆cycloalkyl and C₁₋₆alkyl-S(O)ₖ—, wherein k is 0, or 2, and
wherein items (7), (9), (11) and (12) above and aryl portion of items (8), (13) and (14) above and the HET portion of item (10) above are optionally substituted with from one to three substituents independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy and
(o) CN,
wherein items (d) to (g) above and the alkyl portions of item (k) above are optionally substituted from one to three substituents independently selected from the group consisting of: halo, OR¹³ and N(R¹⁴)₂, and
wherein items (h), (i), (j), (l) and (m) above and the aryl portions of items (k) and (n) above are optionally substituted from one to three substituents independently selected from the group consisting of: halo, OR¹³ and C₁₋₄alkyl,
each R¹³ and R¹⁴ is independently selected from the group consisting of hydrogen and C₁₋₄alkyl, optionally substituted from one to three halo groups;
R¹⁶ and each R¹¹ are independently selected from the group consisting of:
(1) hydrogen,
(2) halo,
(3) methyl,
(4) methoxy, and
(5) hydroxy;
Y₁ and Y₂ are each selcected from the group consisting of:
(1) hydrogen,
(2) hydroxy,
(3) halo,
(4) methyl,
(5) —NO₂,
(6) —CN,
(6) mono, di or tri halo substituted methyl,
X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);

13. A compound according to claim 12 wherein Y₁, R¹¹ and R¹⁶ are each hydrogen.

14. A compound according to claim 12 of Formula Ic: 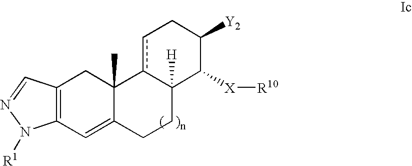
wherein
n is 0 or 1,
R¹ is phenyl, optionally mono or di-substituted with halo;
R¹⁰ is selected from the group consisting of:
(1) C₁₋₆alkyl,
(2) C₂₋₆alkenyl,
(3) C₂₋₆akynyl,
(4) C₃₋₆cycloalkyl,
(5) C₁₋₆alkoxy,
(6) C₁₋₆alkyl-S(O)ₖ—, wherein k is 0, 1 or 2,
(7) aryl,
(8) aryl C₁₋₆alkyl,
(9) HET,
(10) —C₁₋₆alkyl-HET,
(11) aryloxy,
(12) aroyloxy,
(13) aryl C₂₋₆alkenyl,
(14) aryl C₂₋₆alkynyl,
(15) hydrogen, and
(16) hydroxy
wherein items (1) to (6) above and the alkyl portions of items (8) and (10) above and the alkenyl portion of item (13) above and the alkynyl portion of item (14) above are optionally substituted with from one to three substituents independently selected from the group consisting of: halo, OR¹³, N(R¹⁴)₂, C₃₋₆cycloalkyl and C₁₋₆alkyl-S(O)ₖ—, wherein k is 0, 1 or 2, and
wherein items (7), (9), (11) and (12) above and aryl portion of items (8), (13) and (14) above and the HET portion of item (10) above are optionally substituted with from one to three substituents independently selected from the group consisting of:
(a) halo,
(b) OR¹³,
(c) N(R¹⁴)₂,
(d) C₁₋₆alkyl,
(e) C₂₋₆alkenyl,
(f) C₂₋₆akynyl,
(g) C₁₋₆alkyl-S(O)ₖ—, wherein k is 0, 1 or 2,
(h) aryl,
(i) aryl-S(O)ₖ—, wherein k is 0, 1 or 2,
(j) HET,
(k) aryl C₁₋₆alkyl,
(l) aroyl,
(m) aryloxy,
(n) aryl C₁₋₆alkoxy and
(o) CN,
wherein items (d) to (g) above and the alkyl portions of item (k) above are optionally substituted with from one to three substituents independently selected from the group consisting of: halo, OR¹³ and N(R¹⁴)₂, and
wherein items (h), (i), (j), (l) and (m) above and the aryl portions of items (k) and (n) above are optionally substituted with from one to three substituents independently selected from the group consisting of: halo, OR¹³ and C₁₋₄alkyl,
each R¹³ and R¹⁴ is independently selected from the group consisting of hydrogen and C₁₋₄alkyl, optionally substituted with from one to three halos;
R¹⁶ and each R¹¹ are independently selected from the group consisting of:
(1) hydrogen,
(2) halo,
(3) methyl,
(4) methoxy, and
(5) hydroxy;
Y₁ and Y₂ are each selcected from the group consisting of:
(1) hydrogen,
(2) hydroxy,
(3) halo,
(4) methyl,
(5) —NO₂,
(6) —CN,
(6) mono, di or tri halo substituted methyl,
X is a bond, —C(O), —N(R¹⁴)—, —N(R¹⁴)—C(O)—, —C(O)—N(R¹⁴)—, —N(R¹⁴)—S(O)ₖ—, —N(R¹⁴)—C(O)—NH— or —S(O)ₖ—N(R¹⁴);

15. The compound according to claim 13 wherein
X is a bond, —C(O), —N(R¹⁴)—, —N(R¹⁴)—C(O)—, —C(O)—N(R¹⁴)—, —N(R¹⁴)—C(O)—NH—;
R¹³ and R¹⁴ are each independently selected from hydrogen or methyl; and
R¹⁰ are each independently selected from the group consisting of:
(1) C₁₋₄alkyl,
(2) C₂₋₄alkenyl,
(3) C₂₋₄akynyl,
(4) C₃₋₆cycloalkyl,
(5) C₁₋₄alkoxy,
(6) aryl,
(7) aryl C₁₋₄alkyl,
(8) HET,
(9) —C₁₋₄alkyl-HET,
(10) aryloxy,
(11) aroyloxy,
(12) aryl C₂₋₄alkenyl,
(13) aryl C₂₋₆alkynyl,
wherein items (1) to (5) above and the alkyl portions of items (7) and (9) above and the alkenyl portion of item (12) above and the alkynyl portion of item (13) above are optionally substituted with from to three substituents independently selected from the group consisting of: halo, OR¹³, N(R¹⁴)₂, C₃₋₆cycloalkyl and C₁₋₆alkyl-S(O)ₖ—, wherein k is 0, 1 or 2, and
wherein items (6), (8), (10) and (11) above and aryl portion of items (7), (12) and (13) above and the HET portion of item (9) above are optionally substituted with from one to three substituents independently selected from the group consisting of:
(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
wherein items (d) to (f) above and the alkyl portions of item (i) above are optionally substituted with from one to three substituents independently selected from the group consisting of: halo, OR¹³ and N(R¹⁴)₂, and
wherein items (g), (h), (j) and (k) above and the aryl portions of items (i) and (l) above are optionally substituted with from one to three substituents independently selected from the group consisting of: halo, OR¹³ and C₁₋₄alkyl.

16. The compound according to claim 15 wherein
X is a bond, —C(O), —N(R¹⁴)—, —N(R¹⁴)—C(O)—, —C(O)—N(R¹⁴)—, —N(R¹⁴)—C(O)—NH—;
R¹³ and R¹⁴ are each independently from hydrogen or methyl; and
R¹⁰ are each independently selected from the group consisting of:
(1) C₃₋₆cycloalkyl,
(2) aryl,
(3) aryl C₁₋₄alkyl,
(4) HET,
(5) —C₁₋₄alkyl-HET,
(6) aryl C₂₋₄alkenyl,
wherein item (1) above and the alkyl portions of items (3) and (5) above and the alkenyl portion of item (8) above are optionally substituted with from one to three substituents independently selected from the group consisting of: halo, OR¹³, N(R¹⁴)₂, and
wherein the aryl portion of items (2), (3), (6) and the HET portion of item (4) and (5) above are optionally substituted from one up to the maximum number of substitutable positions with a substituent independently selected from the group consisting of:
(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
wherein items (d) to (f) above and the alkyl portions of item (i) above are optionally substituted from one up to the maximum number of substitutable positions with a substituent independently selected from the group consisting of: halo, OR¹³ and N(R¹⁴)₂, and
wherein items (g), (h), (j) and (k) above and the aryl portions of items (i) and (l) above are optionally substituted from one up to the maximum number of substitutable positions with a substituent independently selected from the group consisting of: halo, OR¹³ and C₁₋₄alkyl.

17. The compound according to claim 16 wherein
R¹⁰ are each independently selected from the group consisting of:
(1) C₃₋₆cycloalkyl,
(2) aryl,
(3) aryl C₁₋₄alkyl,
(4) HET,
(5) —C₁₋₄alkyl-HET,
(6) aryl C₂₋₄alkenyl,
wherein item (1) above and the alkyl portions of items (3) and (5) above and the alkenyl portion of item (8) above are optionally substituted with from one to three substituents independently selected from the group consisting of: halo, OR¹³, N(R¹⁴)₂, and
wherein the HET portion of item (4) and (5) are optionally substituted with from one to three substituents selected from the group consisting of C₁₋₄alkyl and aryl, and wherein the aryl portion of items (2), (3), (6) above are optionally substituted with from one to three substituents independently selected from the group consisting of:
(a) halo,
(b) OR¹³,
(c) N(R¹⁴)₂,
(d) C₁₋₄alkyl,
(e) C₂₋₄alkenyl,
(f) C₂₋₄akynyl,
(g) aryl,
(h) HET,
(i) aryl C₁₋₆alkyl,
(j) aroyl,
(k) aryloxy,
(l) aryl C₁₋₆alkoxy and
(m) CN,
wherein items (d) to (f) above and the alkyl portions of item (i) above are optionally substituted from one up to the maximum number of substitutable positions with a substituent independently selected from the group consisting of: halo, OR¹³ and N(R¹⁴)₂, and
wherein items (g), (h), (j) and (k) above and the aryl portions of items (i) and (l) above are optionally substituted from one up to the maximum number of substitutable positions with a substituent independently selected from the group consisting of: halo, OR¹³ and C₁₋₄alkyl.

18. The compound according to claim 3 wherein Y₂ is CF₃.

19. The compound according to claim 18 wherein R¹⁰ is selected from the group consisting of:
(1) phenyl,
(2) benzyl, and
(3) HET, wherein HET is a 5-membered aromatic or non-aromatic monocyclic ring containing 1-3 heteroatoms selected from O, S and N,
wherein groups (1) to (3) above are optionally substituted with 1 to 3 substituents independently selected from the group consisting of:
(a) halo,
(b) C₁₋₄alkyl, optionally substituted with hydroxy or 1 to 3 halo groups,
(c) C₁₋₄alkoxy, optionally substituted with 1 to 3 halo groups,
(d) NH₂,
(e) hydroxy, and
(e) phenyl or benzyl.

20. The compound according to claim 3 wherein Y₂ is hydrogen, X is a bond and R¹⁰ is HET, wherein HET is a 5-membered aromatic or non-aromatic monocyclic ring containing 1-3 heteroatoms selected from O, S and N.

21. The compound according to claim 20 wherein HET is selected from oxazolyl and imidazolyl.

22. A compound selected from the group consisting of:

| | |
|---|---|
| 1 | 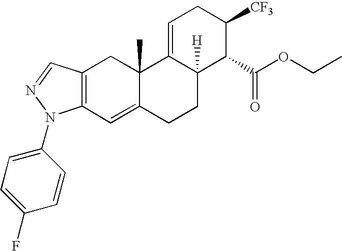 |
| 2 | 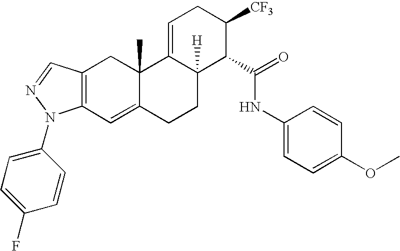 |
| 3 | 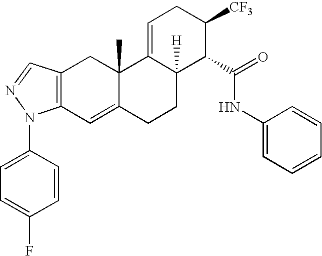 |
| 4 | 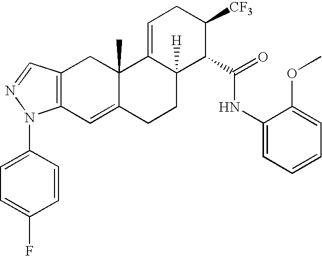 |
| 5 | 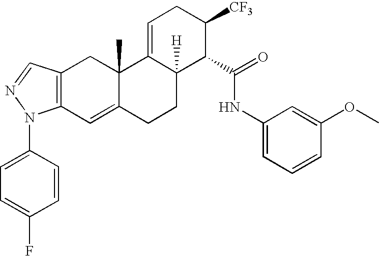 |
| 6 | 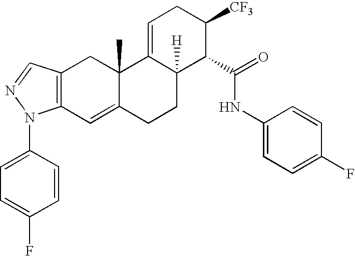 |
| 7 | 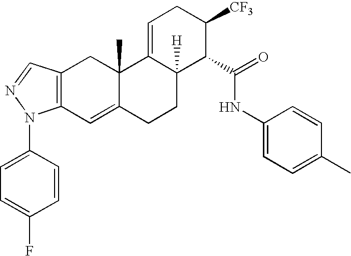 |
| 8 | 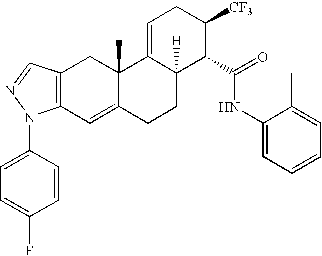 |
| 9 | 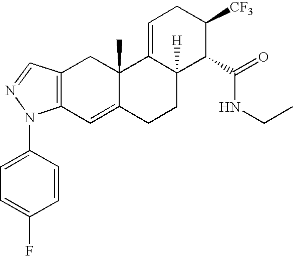 |
| 10 | 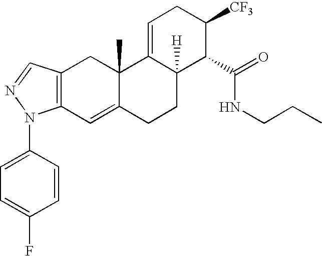 |
| 11 | 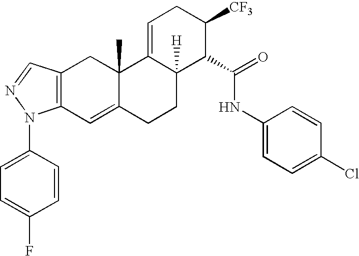 |
| 12 | 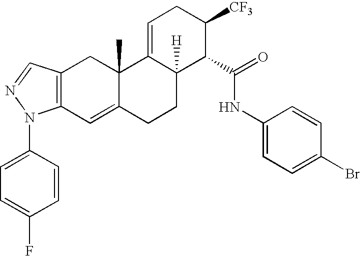 |
| 13 | 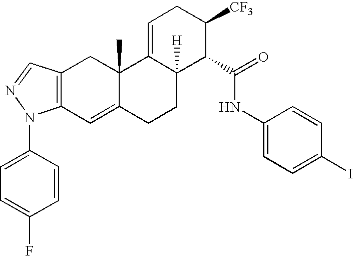 |
| 14 | 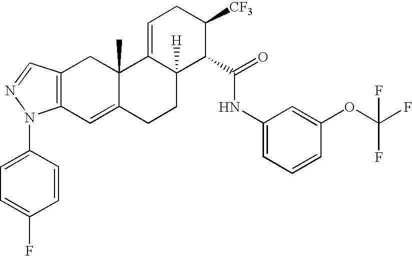 |
| 15 | 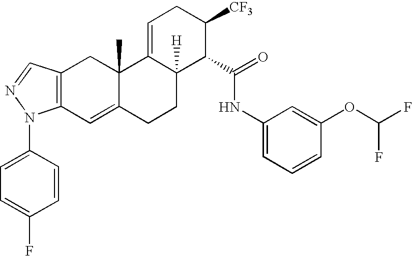 |
| 16 | 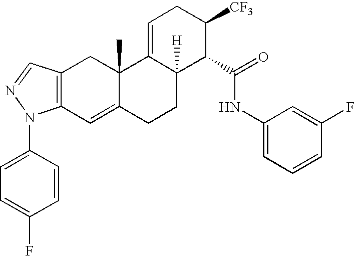 |
| 17 | 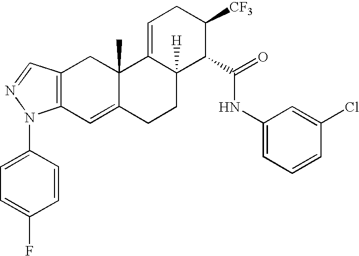 |
| 18 | 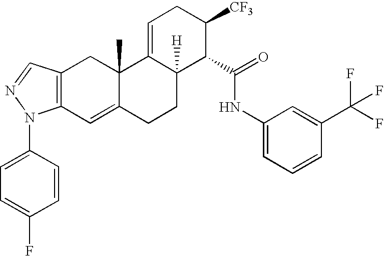 |
| 19 | 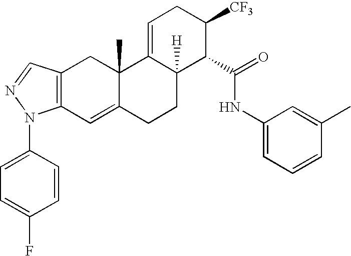 |
| 20 | 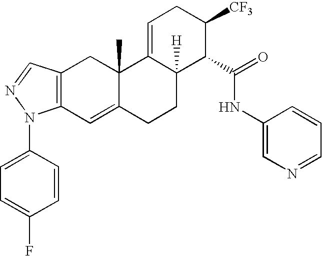 |
| 21 | 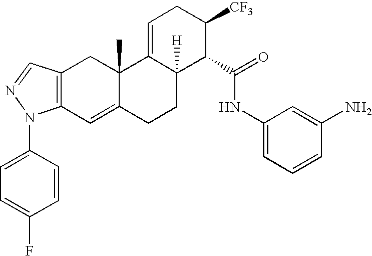 |
| 22 | 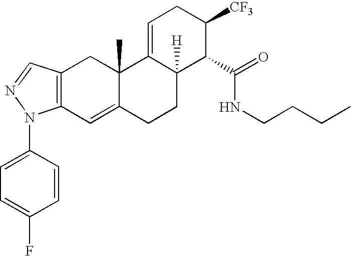 |
| 23 | 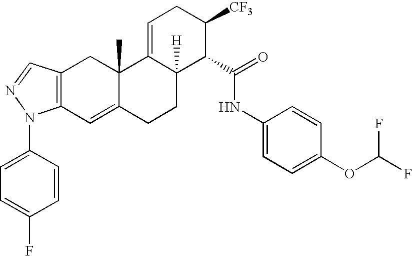 |
| 24 | 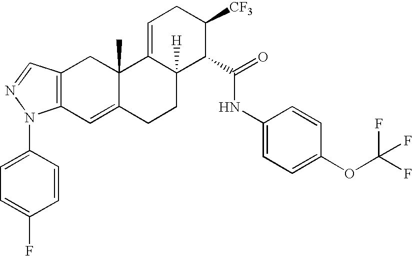 |
| 25 | 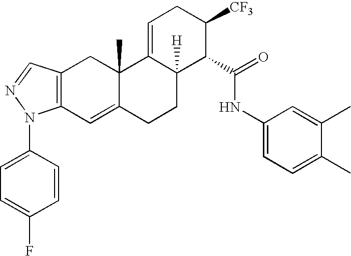 |
| 26 | 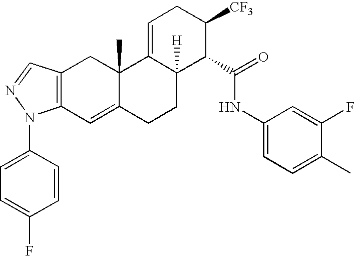 |
| 27 | 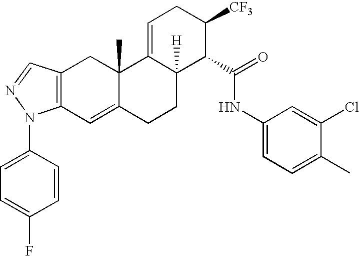 |
| 28 | 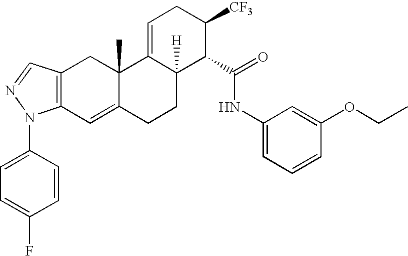 |
| 29 | 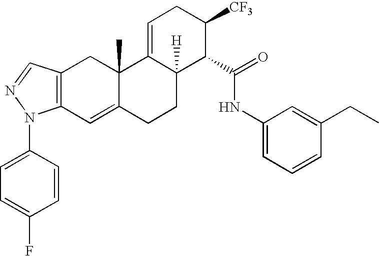 |
| 30 | 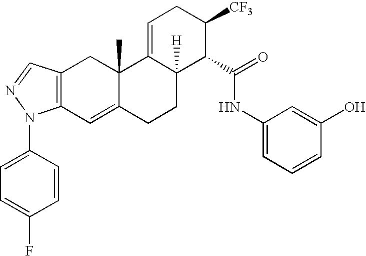 |
| 31 | 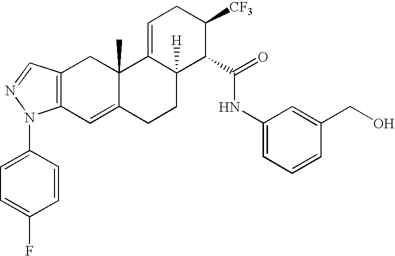 |
| 32 | 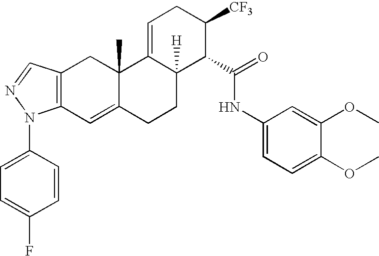 |
| 33 | 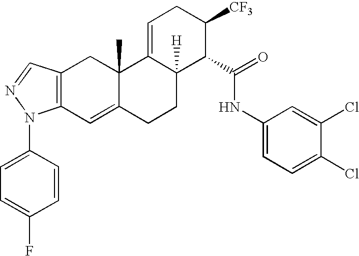 |
| 34 | 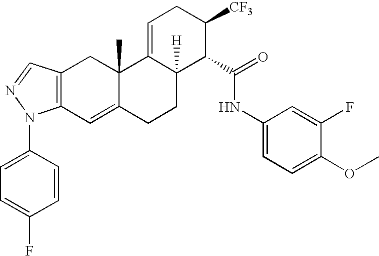 |
| 35 | 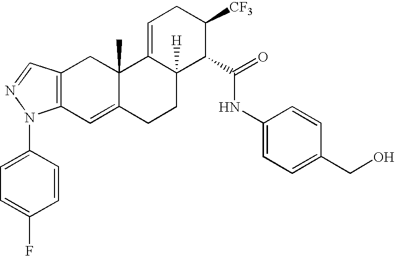 |
| 36 | 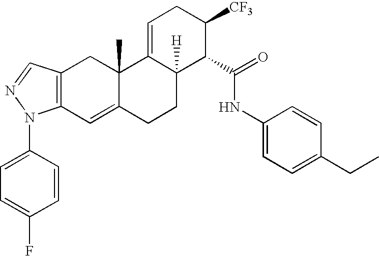 |
| 37 | 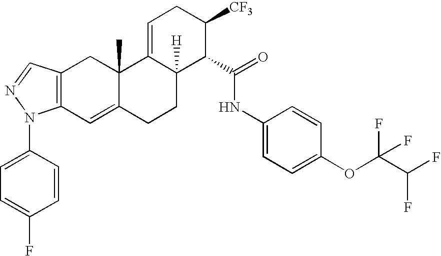 |
| 38 | 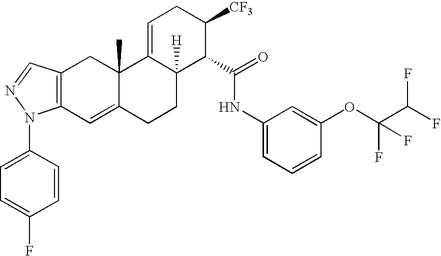 |
| 39 | 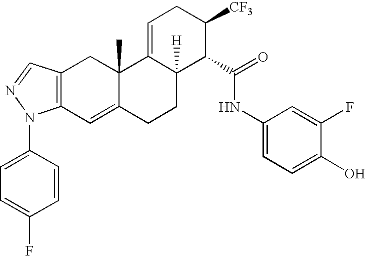 |
| 40 | 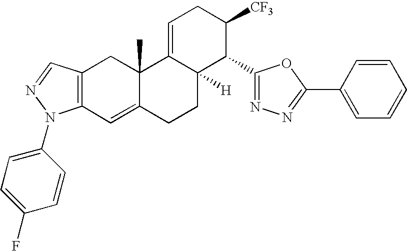 |
| 41 | 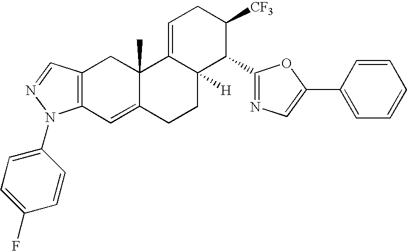 |
| 42 | 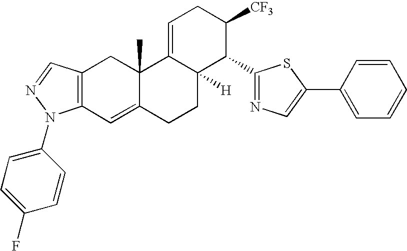 |
| 43 | 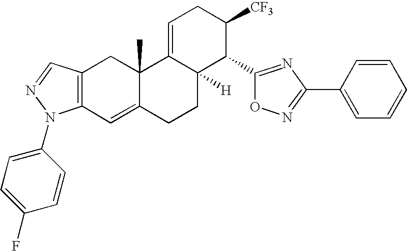 |
| 44 | 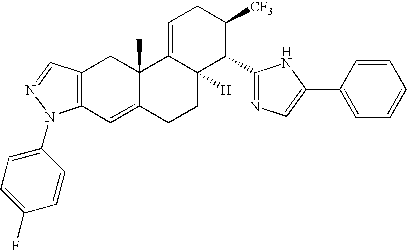 |
| 45 | 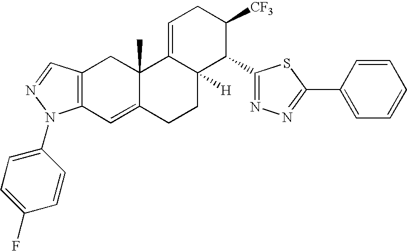 |
| 46 | 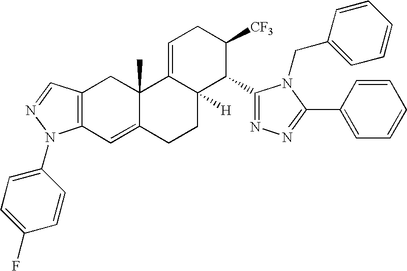 |
| 47 | 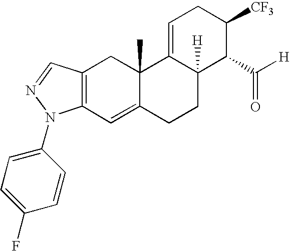 |
| 48 | 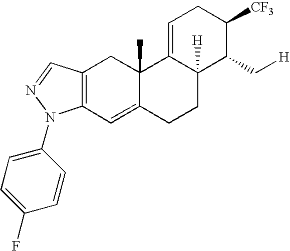 |
| 49 | 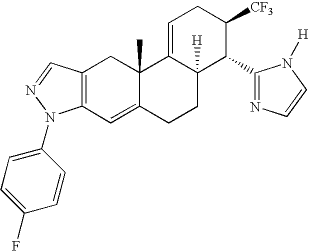 |
| 50 | 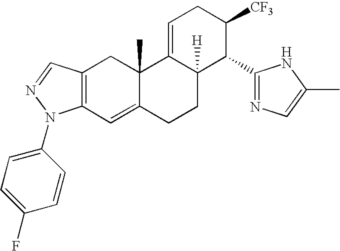 |
| 51 | 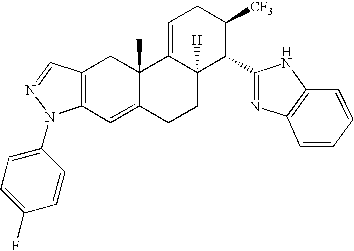 |
| 52 | 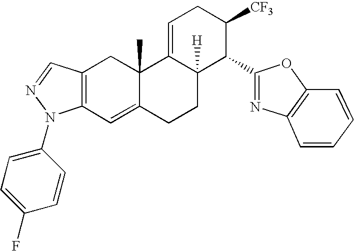 |
| 53 | 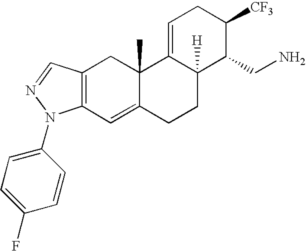 |
| 54 | 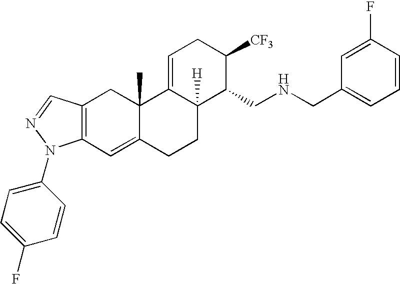 |
| 55 | 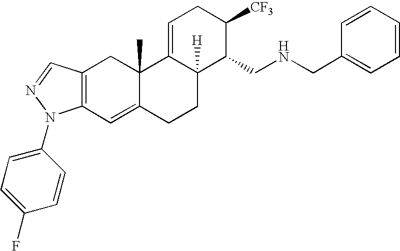 |
| 56 | 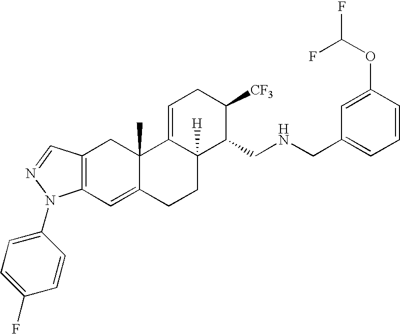 |
| 57 | 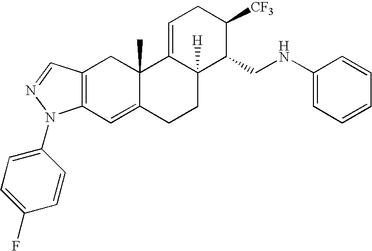 |
| 58 | 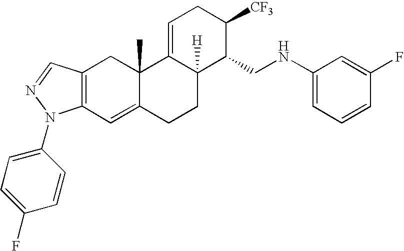 |
| 59 | 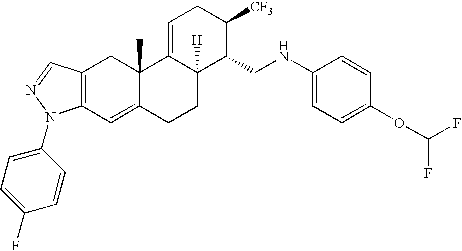 |
| 60 | 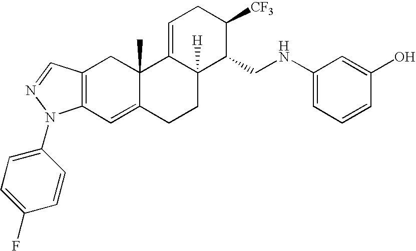 |
| 61 | 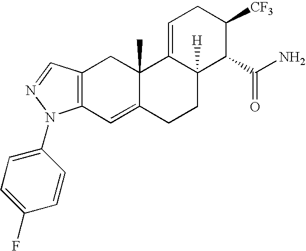 |
| 62 | 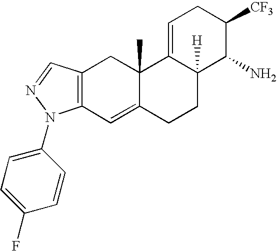 |
| 63 | 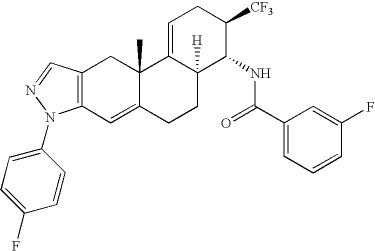 |
| 64 | 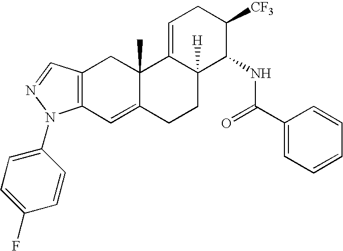 |
| 65 | 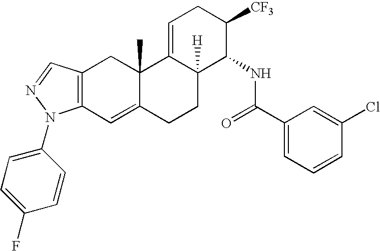 |
| 66 | 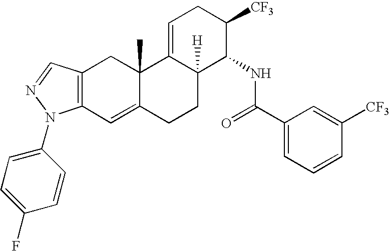 |
| 67 | 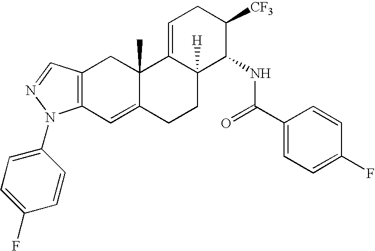 |
| 68 | 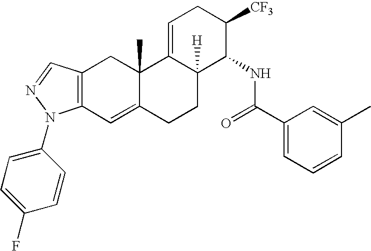 |
| 69 | 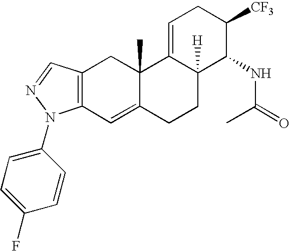 |
| 70 | 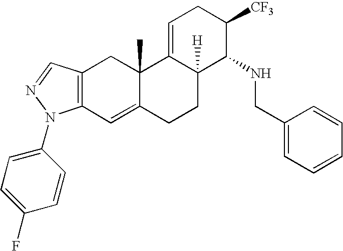 |
| 71 | 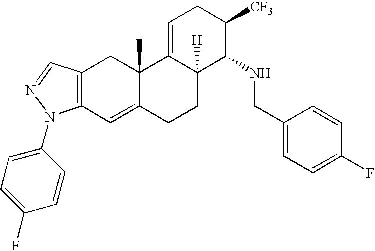 |
| 72 | 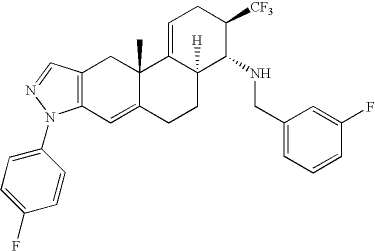 |
| 73 | 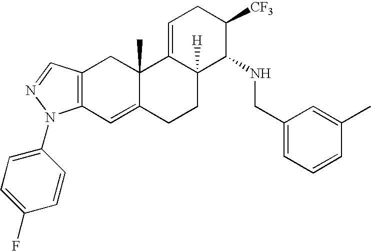 |
| 74 | 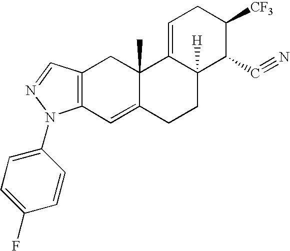 |
| 75 | 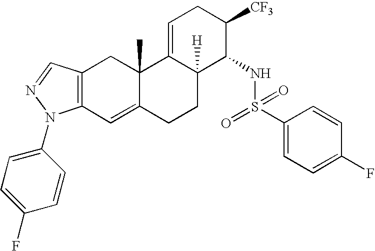 |
| 76 | 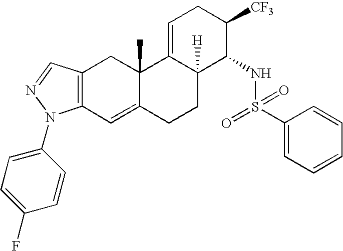 |
| 77 | 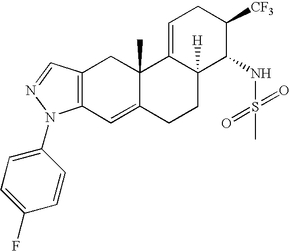 |
| 78 | 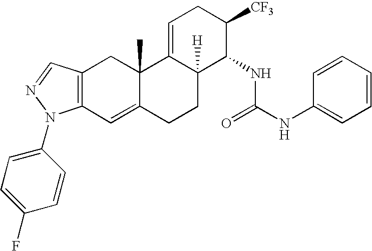 |
| 79 | 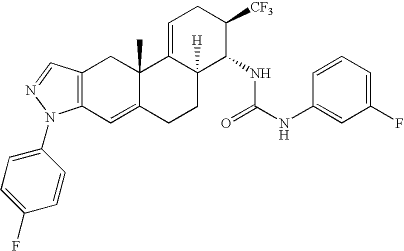 |
| 80 | 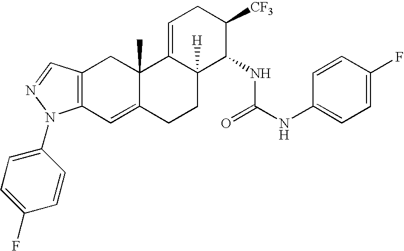 |
| 81 | 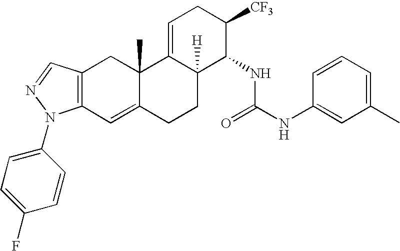 |
| 82 | 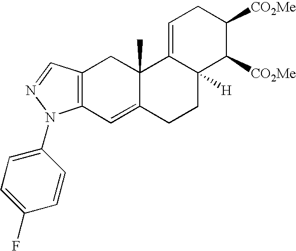 |
| 83 | 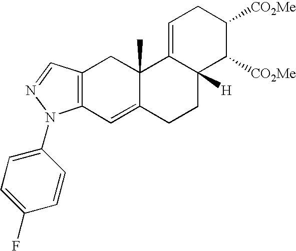 |
| 84 | 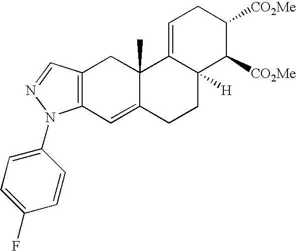 |
| 85 | 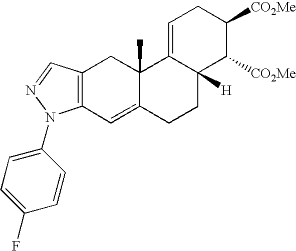 |
| 86 | 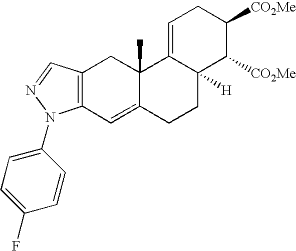 |
| 87 | 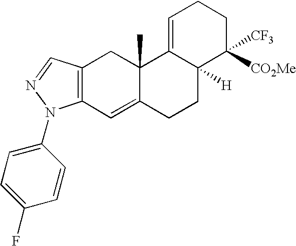 |
| 88 | 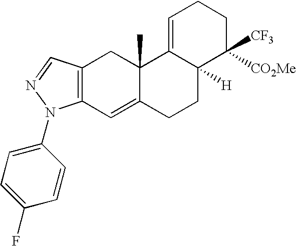 |
| 89 | 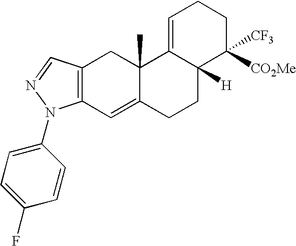 |
| 90 | 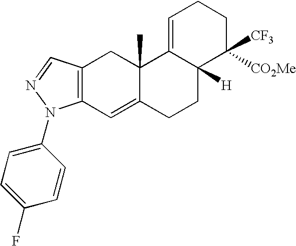 |
| 91 | 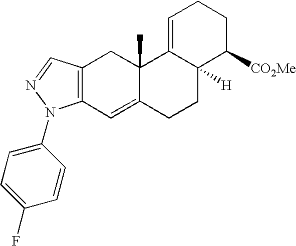 |
| 92 | 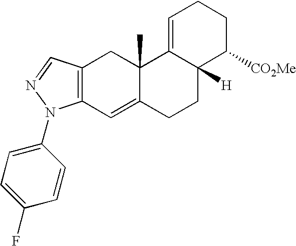 |
| 93 | 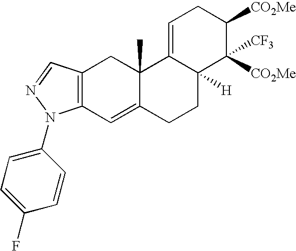 |
| 94 | 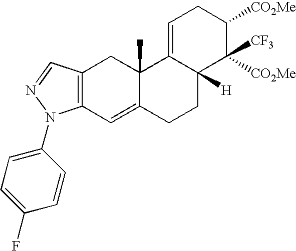 |
| 95 | 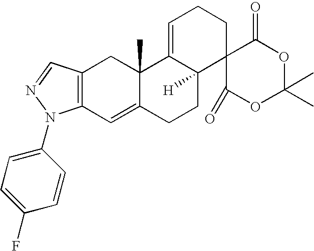 |
| 96 | 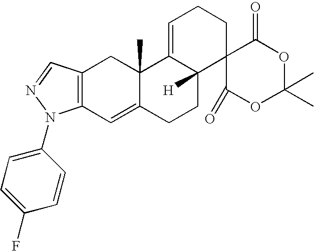 |
| 97 | 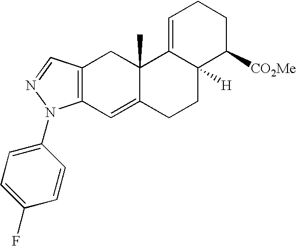 |
| 98 | 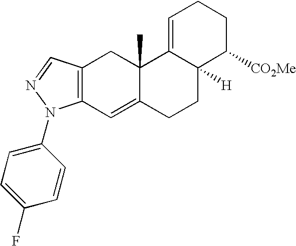 |
| 99 | 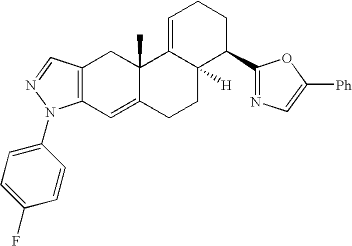 |
| 100 | 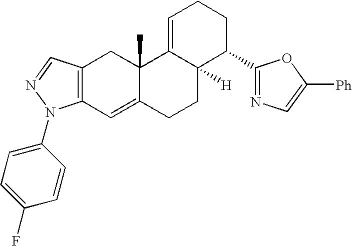 |
| 101 | 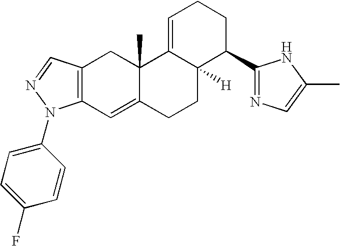 |
| 102 | 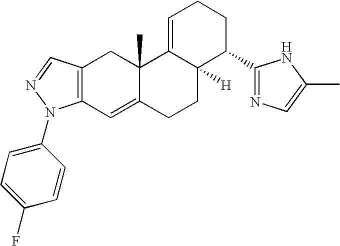 |
| 103 | 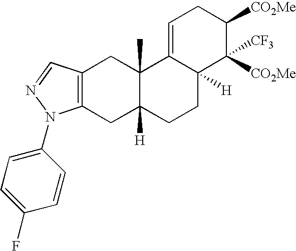 |
| 104 | 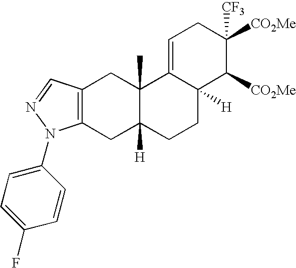 |
| 105 | 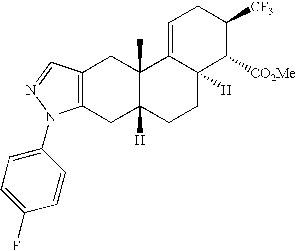 |
| 106 | 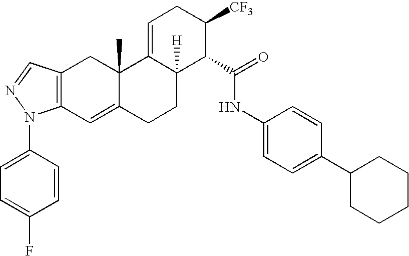 |
| 107 | 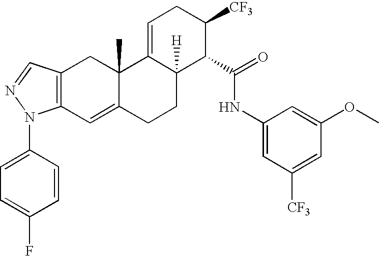 |
| 108 | 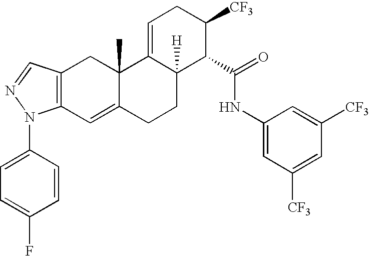 |
| 109 | 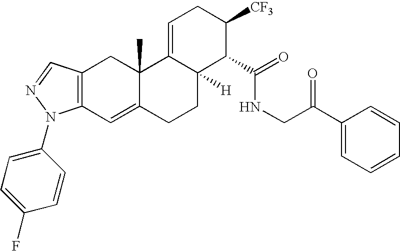 |
| 110 | 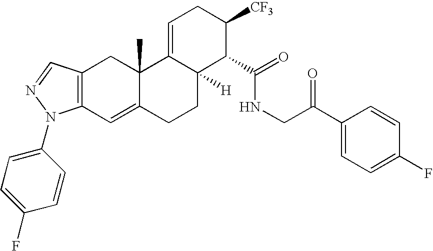 |
| 111 | 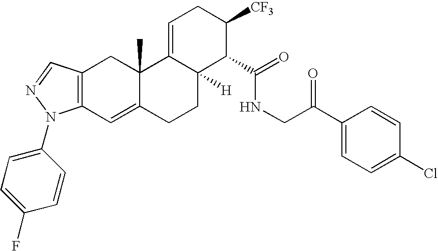 |
| 112 | 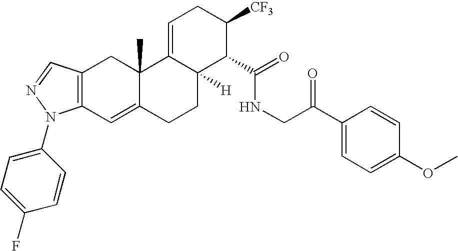 |
| 113 | 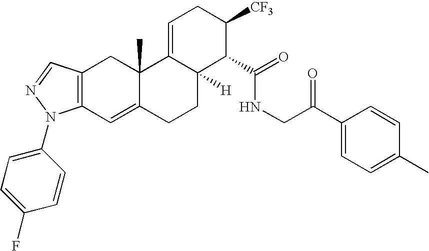 |
| 114 | 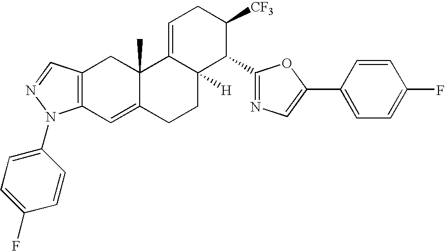 |
| 115 | 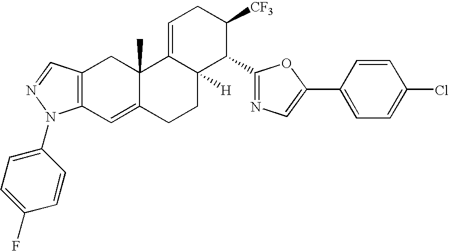 |
| 116 | 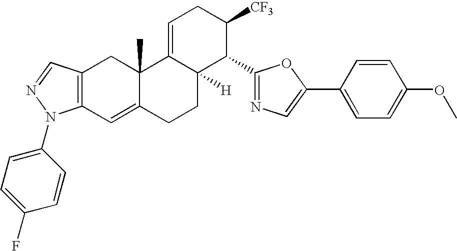 |
| 117 | 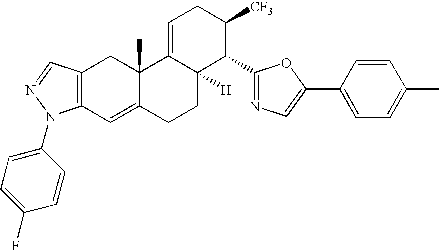 |
| 118 | 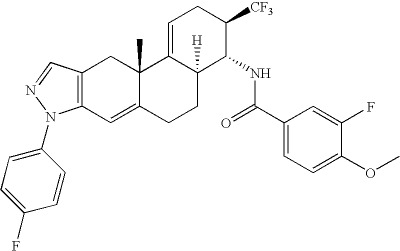 |
| 119 | 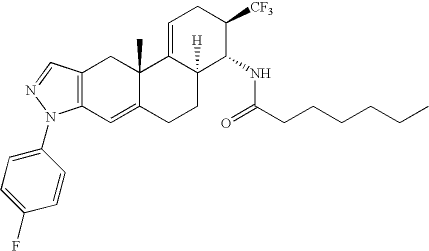 |
| 120 | 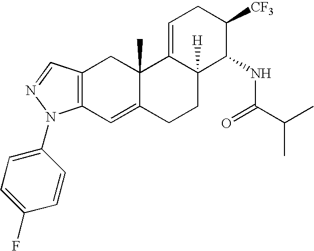 |
| 121 | 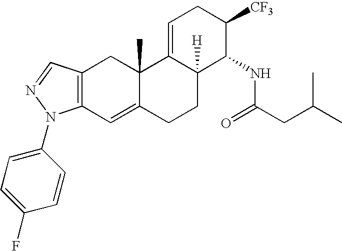 |
| 122 | 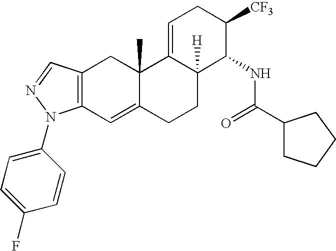 |
| 123 | 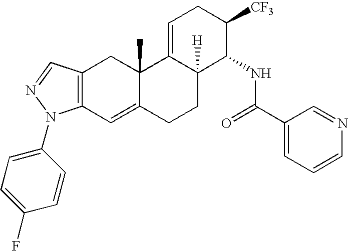 |
| 124 | 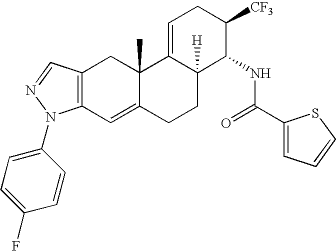 |
| 125 | 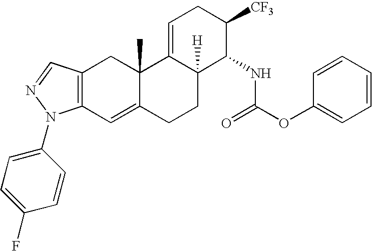 |
| 126 | 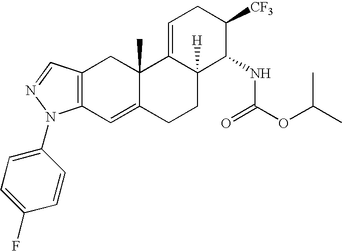 |
| 127 | 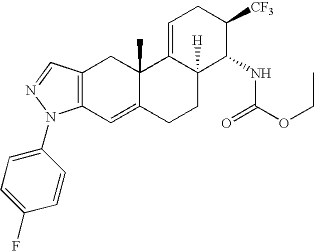 |
| 128 | 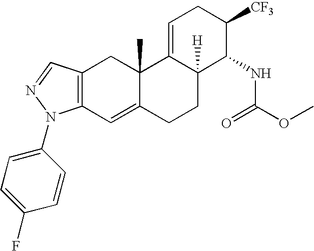 |
| 129 | 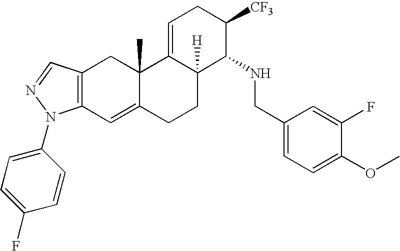 |
| 130 | 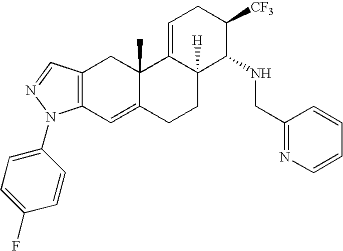 |
| 131 | 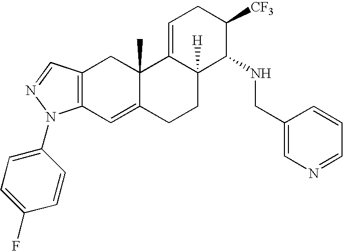 |
| 132 | 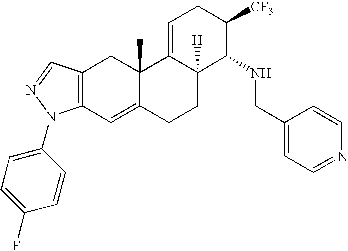 |
| 133 | 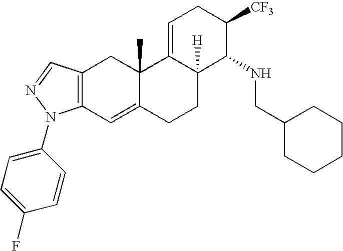 |
| 134 | 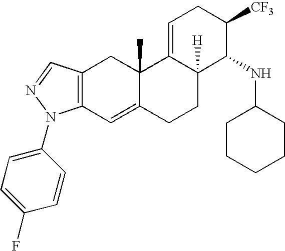 |
| 135 | 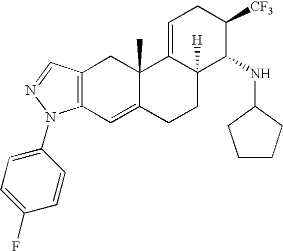 |
| 136 | 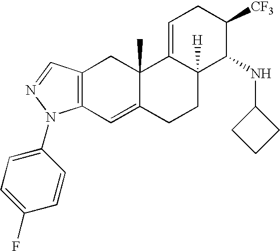 |
| 137 | 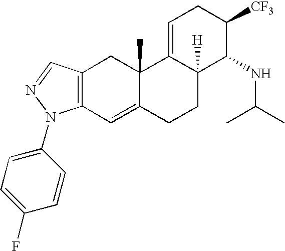 |
| 138 | 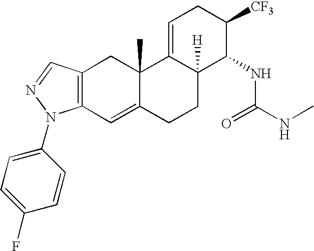 |
| 139 | 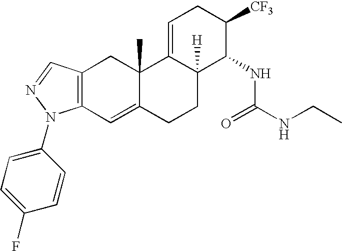 |
| 140 | 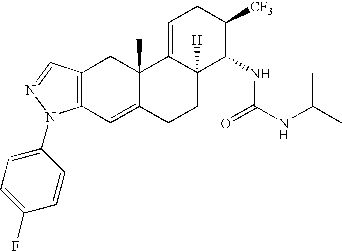 |

23. A pharmaceutical composition comprising a compound according to claim 1 in combination with a pharmaceutically acceptable carrier.

24. A method for treating a glucocorticoid receptor mediated disease or condition in a mammalian patient in need of such treatment comprising administering the patient a compound according to claim 1 in an amount that is effective for treating the glucocorticoid receptor mediated disease or condition.

25.-28. (canceled)

29. A compound according to claim 1 of Formula Id 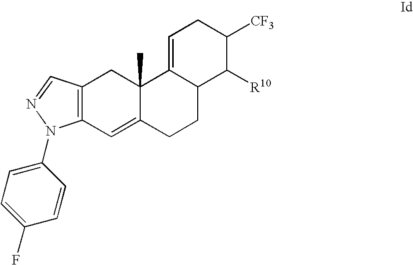
or a pharmaceutically acceptable salt thereof, wherein
R¹⁰ is a 5-membered aromatic or non-aromatic mono-cyclic ring containing 1-3 heteroatoms selected from O, S, and N, and
R¹⁰ is mono-substituted with phenyl, wherein phenyl is optionally substituted with 1-3 substituents independently selected from halo, C₁₋₄alkyl and C₁₋₄alkoxy.

30. The compound according to claim 29 wherein R¹⁰ is oxazolyl, oxadiazolyl or thiazolyl.

31. (canceled)
